SEQUENCE LISTING (25 sequences)

SEQ ID NO: 1 - protein, 121 aa, Homo sapiens
EVQLVESGGGLVQPGTSLRLSCAASGFTFDDYAMHWVRQAPGKGLEWVSGITWNSYNIDYADSVKGRFTISRDNAKNSLYLQMNSLRAEDTALYYCAKDDDYSNYVYFDYWGQGTLVTVSS

SEQ ID NO: 2 - protein, 8 aa, Homo sapiens
GFTFDDYA

SEQ ID NO: 3 - protein, 8 aa, Homo sapiens
ITWNSYNI

SEQ ID NO: 4 - protein, 14 aa, Homo sapiens
AKDDDYSNYVYFDY

SEQ ID NO: 5 - protein, 117 aa, Homo sapiens
EVQLVESGGGLVQPGGSLRLSCAASGFIVTTNYMTWLRQAPGKGLEWVSLIYSSGHTYYADSVKGRFTISRHNSKNTLYLQMDSLRAEDTAVYYCASAFAADVFDIWGQGTMVTVSS

SEQ ID NO: 6 - protein, 8 aa, Homo sapiens
GFIVTTNY

SEQ ID NO: 7 - protein, 7 aa, Homo sapiens
IYSSGHT

SEQ ID NO: 8 - protein, 11 aa, Homo sapiens
ASAFAADVFDI

SEQ ID NO: 9 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 10 - protein, 6 aa, Homo sapiens
QSISSY

SEQ ID NO: 11 - skipped

SEQ ID NO: 12 - protein, 10 aa, Homo sapiens
QQSYSTPPIT

SEQ ID NO: 13 - protein, 1408 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 14 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 15 - protein, 934 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEVGFLHSSHDVNKEASVIMLFSGLK

SEQ ID NO: 16 - protein, 281 aa, Homo sapiens
KEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKR

SEQ ID NO: 17 - protein, 1101 aa, Homo sapiens
STKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 18 - protein, 1082 aa, Homo sapiens
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 19 - protein, 626 aa, Homo sapiens
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEVGFLHSSHDVNKEASVIMLFSGLK

SEQ ID NO: 20 - protein, 650 aa, synthetic construct
  REGION 1..650: note = synthetic
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAEQKLISEEDLEQKLISEEDLHHHHHH

SEQ ID NO: 21 - protein, 838 aa, synthetic construct
  REGION 1..838: note = synthetic
TKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISTWWKEPLNIVSFLFCFASGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAMEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGKE

SEQ ID NO: 22 - protein, 936 aa, Homo sapiens
ECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTEQKLISEEDLGGEQKLISEEDLHHHHHH

SEQ ID NO: 23 - protein, 13 aa, Homo sapiens
VRRLKETKDGFMF

SEQ ID NO: 24 - protein, 11 aa, Homo sapiens
LARQIGASLND

SEQ ID NO: 25 - protein, 35 aa, Homo sapiens
FIKGDLTIANLGTSEGRFMQVVVSRSGPSTPHVNF

(19) United States
(12) Patent Application Publication
(10) Pub. No.: US 2024/0084041 A1
(43) Pub. Date: Mar. 14, 2024

(54) METHODS OF TREATING NON-SMALL CELL LUNG CANCER USING MESENCHYMAL EPITHELIAL TRANSITION FACTOR (MET)-TARGETED AGENTS

(71) Applicant: Regeneron Pharmaceuticals, Inc., Tarrytown, NY (US)

(72) Inventors: Javier PEREZ, Ardsley, NY (US); Petra RIETSCHEL, Hartsdale, NY (US); Mario Antonio NAVAS, West Harrison, NY (US); Anita BOYAPATI, Chappaqua, NY (US)

(21) Appl. No.: 18/459,221

(22) Filed: Aug. 31, 2023

Related U.S. Application Data
(60) Provisional application No. 63/519,798, filed on Aug. 15, 2023.
(60) Provisional application No. 63/374,364, filed on Sep. 1, 2022.

(51) Int. Cl. C07K 16/46; A61P 35/04

(52) U.S. Cl. CPC C07K 16/468; A61P 35/04; C07K 2317/31; C07K 2317/565

(57) ABSTRACT

The present disclosure provides methods for treating or inhibiting the growth of MET associated non-small cell lung cancer comprising selecting a subject with cancer and administering a therapeutically effective amount of a MET x MET bispecific antibody where each binding arm of the bispecific antibody interacts with a different MET epitope. In certain embodiments, the cancer harbors a MET alteration such as an exon 14 alteration in DNA or a deletion that leads to exon 14 skipping, a MET gene amplification, or MET protein overexpression.

### CROSS REFERENE TO RELATED APPLICATIONS

This application claims the benefit under 35 U.S.C. § 119(e) of U.S. Provisional Application Ser. No. 63/519,798, filed Aug. 15, 2023; and U.S. Provisional Application Ser. No. 63/374,364, filed Sep. 1, 2022, which are herein incorporated by reference in their entirety.

### FIELD OF THE INVENTION

The present invention relates to a MET X MET bispecific antibody which specifically binds hepatocyte growth factor receptor (c-Met or MET) and modulates MET signal transduction, for use in treating non-small cell lung cancer (NSCLC).

### SEQUENCE LISTING

An official copy of the sequence listing is submitted concurrently with the specification electronically via EFS-Web as an XML formatted sequence listing named 11305US01_Sequence Listing ST.26, a creation date of Aug. 31, 2023, and a size of about 32,768 bytes. The sequence listing is part of the specification and is herein incorporated by reference in its entirety.

### BACKGROUND

Hepatocyte growth factor (HGF) (a.k.a. scatter factor [SF]) is a heterodimeric paracrine growth factor that exerts its activity by interacting with the HGF receptor (HGFR). HGFR is the product of the c-Met oncogene and is also known as MET. MET is a receptor tyrosine kinase consisting of a transmembrane beta chain linked via a disulfide bridge to an extracellular alpha chain. The binding of HGF to MET activates the kinase catalytic activity of MET resulting in the phosphorylation of Tyr 1234 and Tyr 1235 of the beta chain and subsequent activation of downstream signaling pathways.

MET and/or HGF overexpression, activation, or amplification has been shown to be involved in non-small cell lung carcinoma (NSCLC) (Sierra and Tsao, Ther. Adv. Med. Oncol., 3(1 Suppl): S21—S35, 2011). MET amplification is thought to be a key driver of oncogenesis in NSCLCs. In addition, mutations resulting in exon 14 deletion of MET have been described as oncogenic drivers in a subset of NSCLC. Tumor cell lines having MET gene amplification are highly dependent on MET for growth and survival. Preclinical data implicate MET signaling in resistance to targeted therapies in NSCLC.

Both preclinical and recent clinical results indicate that tumors harboring these genetic alterations respond to MET inhibitors, validating MET as a cancer driver. Various monovalent MET blocking antibodies are in clinical development for the treatment of various cancers (see U.S. Pat. Nos. 5,686,292; 5,646,036; 6,099,841; 7,476,724; 9,260,531; and 9,328,173; and U.S. Patent Application Publications No. 2014/0349310 and 2005/0233960). Those antibodies include onartuzumab (MetMab) and emibetuzumab, (Xiang et al., Clin. Cancer Res. 19(18): 5068-78, 2013, and Rosen et al., Clin. Cancer Res., Published October 10, 2016, doi: 10.1158/1078-0432.CCR-16-1418). Some of these antibodies block ligand-dependent MET signaling, but are not as effective in blocking ligand-independent MET activation.

There remains a significant unmet medical need for improved anti-cancer drugs that potently block both ligand-dependent and ligand-independent MET signaling.

### BRIEF SUMMARY

Provided herein are methods of using bispecific antibodies and fragments thereof, that bind human c-Met receptor protein (MET x MET) for targeting non-small cell lung cancer (NSCLC) cells having a MET alteration such as an exon 14 alteration in DNA or a deletion that leads to exon 14 skipping, a MET gene amplification, or MET protein overexpression. Patients having NSCLC can be treated according to the acceptable standard of care but ultimately may have exhausted all approved available therapies appropriate for the patient as the tumors develop resistance to treatment. Resistance to MET therapy can be intrinsic or acquired, e.g., chemotherapy, immune checkpoint inhibitors, or targeted therapies.

By identifying those patients having a tumor harboring MET alterations and treating those patients with a MET x MET bispecific antibody, surprising effects were achieved as described herein.

Thus, provided herein are methods of treating NSCLC, reducing NSCLC tumor growth, and or causing regression of NSCLC in a subject suffering from a tumor harboring a MET alteration, the method comprising administering to the subject a 250 to 2000 mg dose of a MET x MET bispecific antibody.

Further provided herein are methods of treating or inhibiting the growth of a NSCLC comprising: (1) selecting a subject with a tumor harboring a MET alteration; and (2) administering to the subject (a) a dose of about 250 mg, 500 mg, 750 mg, 1000 mg, 1500 mg, or 2000 mg of a MET x MET bispecific antibody. In some aspects, the administering of step (2) occurs once every 3 weeks.

Still further provided herein are methods for treating a tumor comprising: (a) selecting a subject with NSCLC; (b) determining that the tumor exhibits a MET alteration selected from the group consisting of an Exon 14 alteration in DNA or a deletion that leads to exon 14 skipping, MET gene amplification, and/or Met protein overexpression, comprising (i) obtaining a tissue sample and/or a liquid sample from the subject; and (ii) assessing the tissue sample for MET gene amplification using fluorescent in situ hybridization in tumor tissues or by Next Gen Sequencing in tumor tissues and or ctDNA and/or assessing the tissue sample for Met protein overexpression using immunohistochemistry, and/or assessing the liquid sample for an Exon14 mutation using ctDNA; and, if the tumor exhibits a MET alteration, (c) administering one or more doses of a MET x MET bispecific antibody to the subject in need thereof.

Also provided herein are methods for identifying a candidate for Met x Met anti-tumor therapy, the method comprising obtaining a tissue sample and/or a fluid sample from a subject having NSCLC; and assessing the tissue sample and/or fluid sample for a MET alteration selected from the group consisting of an Exon 14 alteration in DNA or a deletion that leads to exon 14 skipping, MET gene amplification, and/or Met protein overexpression, wherein presence of at least one Met alteration in the tissue sample or fluid sample identifies the subject as a candidate for anti-tumor therapy, wherein the MET x MET anti-tumor therapy comprises a MET x MET bispecific antibody.

MET alterations as provided herein include exon 14 alterations in DNA, MET gene amplification, or MET protein overexpression.

In some aspects, the MET alteration is an exon 14 alteration in DNA or a deletion that leads to exon 14 skipping. An exon 14 alteration in DNA or a deletion that leads to exon 14 skipping includes missense alterations, deletions, splice site changes, and whole exon deletions which result in the skipping of exon 14 of the MET gene. In some aspects, the MET alteration is an exon 14 mutation. In some embodiments, exon 14 mutations can include, but are not limited to, D1010N, D1010fs*19, D1010Y, D1010H, or R1004P.

In some aspects, the MET alteration is a MET gene amplification. In some aspects, a MET gene which is highly amplified has a MET gene copy number (GCN) ≥5 and/or MET to chromosome 7 centromere (MET/CEP7) ratio ≥2 by FISH or MET GCN ≥6 by Next generation sequencing (NGS) in tissues or MET fold change ≥2 in ctDNA.

In some aspects, the MET alteration is MET protein overexpression. MET protein overexpression can be assessed by MET immunohistochemistry (IHC) in tumor tissue that is higher than expression in normal tissue. In some aspects, elevated MET protein expression is measured as an IHC ≥2+ or H score of >150. In some aspects, MET protein highly overexpressed is measured as IHC 3+ or H score of ≥200.

In some aspects, the MET alteration is identified using ctDNA from blood sample, i.e., a liquid biopsy, obtained from the patient prior to treatment.

In some aspects, the MET alteration is identified in a tissue sample, i.e. tumor biopsy, obtained from the patient prior to treatment.

In some aspects, the NSCLC tumor has an EGFR mutation. The mutation can include, but is not limited to L858R, G719S, E709A, E746_A750del, and S752_I759del. In some aspects, the subject is selected as having NSCLC with one or more mutations in the EGFR gene.

In some embodiments, the subject has not received prior anti-MET cancer therapy. In some embodiments, the subject is tyrosine kinase inhibitor (TKI) naïve. In other words, the subject has not received prior treatment with a MET targeted TKI. In some embodiments, the subject has received prior anti-cancer therapy comprising one or more of a MET targeted TKI, a PD-1 inhibitor, an EGFR inhibitor, a PD-L1 inhibitor, surgery, radiation therapy, or chemotherapy. In some aspects, the prior anti-cancer therapy comprises a MET targeted TKI. In some aspects, the prior anti-cancer therapy comprises a PD-1 inhibitor or a PD-L1 inhibitor. In some aspects, the prior anti-cancer therapy comprises an EGFR inhibitor. In some aspects, the subject is resistant or inadequately responsive to, or relapsed after prior therapy.

A MET TKI naïve patient has not been prior treated with any MET TKI; likewise, a patient who has received treatment with a MET TKI is MET TKI experienced. A patient with PD- (L)1 experience has been treated prior with a PD-1 inhibitor (such as, but not limited to, pembrolizumab, nivolumab, cemiplimab, dostarlimab, and retifanlimab) or PD-L1 inhibitor (such as, but not limited to, atezolizumab, avelumab, and durvalumab), and is considered PD-(L)1 experienced; likewise, one with no PD-(L)1 experience (i.e., PD-(L)1 naïve) has not been prior treated with a PD-1 or PD-L1 inhibitor. A patient can be EGFR inhibitor experienced, i.e., has been treated with an EGFR inhibitor. Exemplary EGFR inhibitors include, but are not limited to erlotinib, afatinib, gefitinib, Osimertinib, dacomitinib, cetuximab, panitumumab, dacomitinib, etc.

In some embodiments, the subject is further selected according to one or more of the following criteria:
(i) the subject is MET TKI naïve;
(ii) the subject has a histologically confirmed NSCLC;
(iii) the NSCLC exhibits a MET-exon14 alteration in DNA or a deletion that leads to exon 14 skipping;
(iv) the NSCLC exhibits a MET gene amplification;
(v) the NSCLC exhibits an elevated MET protein expression (IHC ≥2+ or H score of >150);
(vi) the NSCLC exhibits a MET exon 14 alteration in DNA or a deletion that leads to exon 14 skipping and MET TKI experienced;
(vii) the NSCLC exhibits a MET exon 14 alteration in DNA or a deletion that leads to exon 14 skipping and MET TKI naïve;
(viii) the NSCLC exhibits a MET gene highly amplified (MET GCN ≥5 and/or MET/CEP7 ratio >2 by FISH or MET GCN >6 by NGS in tissues or MET fold change ≥2 in ctDNA) and MET TKI naïve;
(ix) the NSCLC exhibits a MET protein highly overexpressed (IHC 3+ or H score of ≥200) and MET TKI naïve; and/or
(x) the NSCLC exhibits a MET gene highly amplified (MET GCN ≥5 and/or MET/CEP7 ratio >2 by FISH or MET GCN >6 by NGS in tissues or MET fold change ≥2 in ctDNA), MET protein highly overexpressed (IHC 3+ or H score of ≥200), and MET TKI naïve.

In some embodiments, the cancer is non-squamous NSCLC. In some embodiments, the cancer is a NSCLC squamous carcinoma. In some aspects, the NSCLC is metastatic, for example, the cancer has metastasized to the brain and/or liver. In some aspects, the NSCLC is unresectable.

Provided herein are methods for monitoring efficacy of treatment with a MET x MET bispecific antibody in a subject having NSCLC with a MET alteration, the method comprising: (i) obtaining a tissue sample and/or a fluid sample from the subject and assessing the tissue sample and/or fluid sample for somatic mutations in one or more genes selected from the group consisting of:
a. an on-target MET Receptor gene mutation that confers resistance to MET TKI's found in MET Ex14 Mut patients with prior TKI Exp and MET gene silencing (loss-of-function);
b. TK driver receptor activation selected from alternative or parallel TK Receptor and ligand gene amplification and TKR activating mutations; and
c. an activating gene mutation in a pathway selected from the group consisting of JAK2/STAT3 pathway, RAS/RAF/MEK/MAPK pathway, PI3K/AKT/MTOR pathway, TP53 mutations, and cell cycle gene amplification;
   (ii) administering a MET x MET bispecific antibody to the subject; and (iii) repeating steps (i) and (ii) over the course of treatment; wherein the acquiring of a mutation by one or more genes is indicative of resistance to therapy and/or indicative of poor prognosis.

In some aspects, the on-target MET Receptor gene mutation is selected from the group consisting of METY1230C, MET D1228H, MET D1228N; and the MET gene silencing (loss-of-function) is selected from somatic mutations in DNMT3A and TET2.

In some aspects, the TKR activating mutation is selected from the group consisting of EGFR L858R, EGFR G719S, EGFR E709A, EGFR E746_A750del, and EGFR S752_I759del.

In some aspects, the JAK2/STAT3 pathway mutation is JAK2 V617F; the RAS/RAF/MEK/MAPK pathway mutation is selected from the group consisting of KRAS G12A/V, GNAS R201H, MKRN-BRAF fusion, BRAF S602Y, RICTOR Amp, and MAP2K1 K57N; the PI3K/AKT/MTOR pathway mutation is selected from the group consisting of PIK3CA H1047L, PIK3CA E545K, PIK3CA E542K, PIK3CA N345K, IDH1 R132L, and MTOR E2338Q; the PI3K/AKT/MTOR pathway amplification is selected from the group consisting of AKT2 Amp and RICTOR Amp; the TP53 mutation is selected from the group consisting of TP53 R280T and TP53 R248Q; and the cell cycle gene amplification is selected from the group consisting of CDK4 Amp, CDK6 Amp, CCND1 Amp, and CCNE1 Amp.

In some aspects, the patient has confirmed MET amplification and MET overexpression, but develops gene amplification in one or more of HGF, EPH, EGFR, BRAF, BCL2L1, PI3KCB, KRAS, AKT2, ATR, VEFGA, FGF, CCND, CCNE, CDK6, RAD21, and MYC. In some aspects, the patient has confirmed MET amplification and MET overexpression, but develops gene deletion in one or more of CDKN2A, CDKN2B, MTAP, and RBM10. In some aspects, the patient has confirmed MET amplification and MET overexpression, but develops one or more of the point mutations provided in Table 15.

In some aspects, the patient has confirmed MET amplification (and not MET overexpression) and can be an EGFR mutant, but develops gene amplification in one or more of EGFR, BRAF, PI3KC2G, KRAS, HGF, EPHA3, ERCC4, RICTOR, RAD21, LYN, MYC, MDM2, CDK 4/6, FgF3/4/19, FGF10, and CCND1. In some aspects, the patient has confirmed MET amplification (and not MET overexpression) and can be an EGFR mutant, but develops gene deletion in one or more of CDKN2A, CDKN2B, MTAP, TEK, and BCOR. In some aspects, the patient has confirmed MET amplification (and not MET overexpression) and can be an EGFR mutant, but develops one or more of the point mutations provided in Table 16.

In some aspects, the patient has confirmed MET Exon 14 alteration and is TKI naïve, but develops gene amplification in one or more of MDM2, EGFR, FGFR1, ERBB3, CDK4, GNA13, MYC, RPTOR, TERC, IKZF1, EZH2, SDHA, SOX, WHSC1L1, and ZNF703. In some aspects, the patient has confirmed MET Exon 14 alteration and is TKI naïve, but develops gene deletion in one or more of CDKN2A, CDKN3A, and MTAP. In some aspects, the patient has confirmed MET Exon 14 alteration and is TKI naïve, but develops one or more of the point mutations provided in Table 17.

In some aspects, the patient has confirmed MET Exon 14 alteration and is TKI experienced, but develops gene amplification in one or more of EGFR, RAF1, PI3KC2G, CDK4, CEBPA, CDKN1A, CARD11, MYC, RICTOR, VEGFA, CD22, DDR1, RAC1, NBN, FGF19, MDM2, NFKBIA, CCND1, INPP4B, PPARG, PMS2, GATA4, SDHA, and RAD21. In some aspects, the patient has confirmed MET Exon 14 alteration and is TKI experienced, but develops gene deletion in one or more of CDKN2A, CDKN3A, and MTAP. In some aspects, the patient has confirmed MET Exon 14 alteration and is TKI experienced, but develops one or more of the point mutations provided in Table 18.

Also provided herein are methods for treating NSCLC in a subject, the method comprising: (i) obtaining a liquid sample from the subject and determining the MET amplification in ctDNA from the liquid sample, and (ii) administering a MET x MET bispecific antibody to the subject; wherein steps (i) and (ii) are repeated once every three weeks, and wherein a loss of MET amplification after step (ii) is repeated is indicative of durable response to treatment.

Provided herein are methods for determining a therapeutically effective amount of a MET x MET bispecific antibody, the method comprising: (i) administering a dosage of the bispecific antibody to a patient in need thereof, and (ii) measuring soluble MET in a blood sample, wherein a maximal increase in soluble MET (sMET) indicates saturation of receptor occupancy and a therapeutically effective amount of the bispecific antibody.

In some aspects, the subject is administered a MET x MET bispecific antibody in an amount of, i.e., a dose of about 250 mg to about 5000 mg, or a dose of about 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 3000 mg, 3500 mg, 4000 mg, 4500 mg, or 5000 mg. In some aspects, the dose is about 250 mg MET x MET bispecific antibody. In some aspects, the dose is about 500 mg MET x MET bispecific antibody. In some aspects, the dose is about 1000 mg MET x MET bispecific antibody. In some aspects, the dose is about 2000 mg MET x MET bispecific antibody. In some aspects, the dose is 250 mg, 500 mg, 1000 mg, or 2000 mg MET × MET bispecific antibody.

In some aspects, the bispecific antibody is administered intravenously, subcutaneously, or intraperitoneally. The bispecific antibody can be administered once every five days, once a week, once every two weeks, once every three weeks, once every four weeks once a month, once every five weeks, once every six weeks, or once every two months. In some aspects, the bispecific antibody is administered once every three weeks. In some aspects, the bispecific antibody is administered three weeks after the immediately preceding dose.

In some aspects, the treatment produces a therapeutic effect selected from the group consisting of delay in tumor growth, reduced metastasis, reduction in tumor cell number, tumor regression, increase in survival, partial response, and complete response. In some aspects, tumor growth is delayed by at least 10 days, or at least 20 days, or at least 30 days, or at least 40 days, or at least 50 days as compared to an untreated subject. In some aspects, the tumor growth is inhibited by at least 10%, by at least 20%, by at least 30%, by at least 40%, by at least 50%, or by at least 60% as compared to an untreated subject.

The MET x MET bispecific antibody comprises a first antigen-binding domain (D1) comprising three heavy chain complementarity determining regions (HCDR1, HCDR2 and HCDR3) within a heavy chain variable region (HCVR) comprising the amino acid sequence that is at least 95% identical to the sequence of SEQ ID NO: 1 and three light chain complementarity determining regions (LCDR1, LCDR2 and LCDR3) within a light chain variable region (LCVR) comprising the amino acid sequence that is at least 95% identical to the sequence of SEQ ID NO: 9; and a second antigen-binding domain (D2) comprising three heavy chain complementarity determining regions (HCDR1, HCDR2 and HCDR3) within a heavy chain variable region (HCVR) comprising the amino acid sequence that is at least 95% identical to the sequence of SEQ ID NO: 5 and three light chain complementarity determining regions (LCDR1, LCDR2 and LCDR3) within a light chain variable region (LCVR) comprising the amino acid sequence that is at least 95% identical to the sequence of SEQ ID NO: 9.

In some aspects, D1 specifically binds a first epitope of human MET.

In some embodiments, D1 comprises an HCDR1 amino acid sequence as set forth in SEQ ID NO: 2; an HCDR2 amino acid sequence as set forth in SEQ ID NO: 3; an HCDR3 amino acid sequence as set forth in SEQ ID NO: 4; an LCDR1 amino acid sequence as set forth in SEQ ID NO: 10; an LCDR2 amino acid sequence as set forth in SEQ ID NO: 11; and an LCDR3 amino acid sequence as set forth in SEQ ID NO: 12.

In some embodiments, D1 comprises an HCVR comprising the amino acid sequence of SEQ ID NO: 1; and an LCVR comprising the amino acid sequence of SEQ ID NO: 9.

In some aspects, D2 specifically binds a second epitope of human MET.

In some embodiments, D2 comprises an HCDR1 amino acid sequence as set forth in SEQ ID NO: 6; an HCDR2 amino acid sequence as set forth in SEQ ID NO: 7; an HCDR3 amino acid sequence as set forth in SEQ ID NO: 8; an LCDR1 amino acid sequence as set forth in SEQ ID NO:10; an LCDR2 amino acid sequence as set forth in SEQ ID NO: 11; and an LCDR3 amino acid sequence as set forth in SEQ ID NO: 12.

In some embodiments, D2 comprises an HCVR comprising the amino acid sequence of SEQ ID NO: 5; and an LCVR comprising the amino acid sequence of SEQ ID NO: 9.

In some embodiments, the MET alteration is an exon 14 alteration in DNA or a deletion that leads to exon 14 skipping, a MET gene amplification, or MET protein overexpression. In some aspects, the MET alteration is an exon 14 alteration in DNA or a deletion that leads to exon 14 skipping. In some aspects, the MET alteration is a MET gene amplification. In some aspects, the MET alteration is MET protein overexpression.

Other embodiments will become apparent from a review of the ensuing detailed description.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A and 1B contain a bar charts depicting the relative growth of EBC-1 cells as a function of treatment with control antibody and a MET x MET bispecific antibody at 0.1 and 1 ug/mL.

FIG. 2 depicts an immunoblot of the expression of MET (and tubulin as a loading control) in Hs746T cells after treatment with a control antibody and a MET x MET bispecific antibody for 2, 6 and 18 hours.

FIG. 3 depicts an immunoblot of pMET, MET, pErk, and tubulin (for loading control) extracted from EBC-1 cells after treatment with a control antibody and a MET x MET bispecific antibody.

FIG. 4 is a line graph depicting the change in EBC-1 tumor volume in cubic millimeters as a function of time in days after implantation of EBC-1 cells in animals treated with control antibody (filled square ▪), MET monovalent antibody (filled circle ●), or MET x MET bispecific antibody (filled diamond ♦)

FIG. 5 is a bar chart depicting the relative cell growth of NCI-H596 cells as a function of treatment with a control antibody (C), a MET x MET bispecific antibody (MM), the MET x MET parental monospecific antibody 1 (M1), the MET x MET parental monospecific antibody 2 (M2), a combination of parental antibodies 1 and 2 (M1M2), and the MET-agonist hepatocyte growth factor (HGF).

FIG. 6 depicts the study flow diagram for the clinical trial using REGN5093.

FIG. 7 is a diagrammatic depiction of the study design showing the dose escalation cohorts and the expansion cohorts.

FIG. 8 depicts patient tumor response characterized by centrally confirmed MET alterations.

FIGS. 9A and 9B depict serum concentrations after the first dose of REGN5093 in the dose escalation cohort, FIG. 9A, and the various expansion cohorts, FIG. 9B. The elimination half-life estimated by non-compartmental analysis over a 3-week dosing interval is 15 days.

FIG. 10 depicts the interaction of the MET x MET bispecific antibody with the MET ectodomain. The Figure also illustrates how MET alterations impact increased MET signaling and ligand independent activation.

FIG. 11 depicts the methods used to confirm MET alterations in ctDNA and tissue, and the exemplary commercial products that can be used in these methods. (CEP7, chromosome 7 centromere; ELISA, enzyme-linked immunosorbent assay; FFPE, formalin-fixed paraffin-embedded; FISH, fluorescent in situ hybridization; FMI, Foundation Medicine, Inc .; GCN, gene copy number; HGF, hepatocyte growth factor; IHC, immunohistochemistry; MET, mesenchymal epithelial transition; sMET, soluble MET; TSO, Trusight Oncology.)

FIG. 12 illustrates the number of patients enrolled in the dose escalation studies and the characteristics and numbers of patients enrolled in the expansion cohorts. (aNSCLC, advanced non-small cell lung cancer; GCN, gene copy number; FIH, first-in-human; IHC, immunohistochemistry; IV, intravenous; MET, mesenchymal epithelial transition; PK, pharmacokinetic; Q3W, every 3 weeks; Q6W, every 6 weeks; TKI, tyrosine kinase inhibitor.)

FIG. 13 provides the response data at the 2000 mg dose for the dose escalation study −2000 mg and the expansion cohorts. Overall response rate was highest for the cohort having patients with an Exon14 skip mutation and where the patients were MET TKI naïve and for the cohort having patients with both MET gene amplification and MET protein overexpression and where the patients were MET TKI naïve.

FIG. 14 shows the tumor response by confirmed MET alteration. MET overexpression—IHC: >75% tumor cells w/ 3+ membrane staining; MET amplification—FISH: GCN ≥5 or MET/CEP7 >2 (tissue); or NGS: GCN ≥6 (tissue); or ≥2.2 fold (ctDNA); MET exon 14 mutation—NGS in tissue or ctDNA. Response to REGN5093 was observed in MET TKI naïve (2L+) aNSCLC patients with MET exon 14 mutation or MET amplification and overexpression regardless of EGFR mutation status. For patients whose tumor tissues were analyzed for MET amplification using two platforms, MET GCN was significantly higher by NGS than by FISH (p=0.0015). (aNSCLC, advanced non-small cell lung cancer; CEP7, chromosome 7 centromere; FISH, fluorescence in situ hybridization; GCN, gene copy number; IHC, immunohistochemistry; MET, mesenchymal epithelial transition; NA, not available or to be determined; NGS, next generation sequencing; PD, progressive disease; PR, partial response; SD, stable disease; TKI, tyrosine kinase inhibitor.)

FIG. 15 provides the response in specific MET altered subgroups (regardless of the presence of other MET alterations) versus overall population. Results are based on small samples sizes and require prospective validation. (CR, complete response; DCR, disease control rate; MET, mesenchymal epithelial transition; ORR, objective response rate; PR, partial response; SD, stable disease; TKI, tyrosine kinase inhibitor.)

FIG. 16 is a chart with the baseline demographics and clinical characteristics of the bypass resistance mutation study population. The study population had a median age of 66 years, 53.8% were male, 70.5% were Asian, and patients had received a median of 2.5 prior lines of therapy. (ECOG, Eastern Cooperative Oncology Group; EGFR, epidermal growth factor receptor; PS, performance status.)

FIG. 17 depicts the differences in detection of somatic variants in tissue versus ctDNA. Tumor profiling at baseline of 2L+ aNSCLC identified somatic variants with known functional significance. Venn diagram shows the total number of baseline somatic variants identified by NGS using FMI-Dx (tissue; n=51 patients) and FMI-L (ctDNA; n=38 patients) panels (324 genes); genes are grouped by type of alteration and type of clinical sample (ctDNA, tissue, or both). CNVs, including gene amplifications and deletions, were detected more readily in tissue while more NSVs (single nucleotide variations, splice variants, and gene fusions) were detected in ctDNA. Thus, bypass gene detection of NSVs and CNVs in ctDNA can, in some embodiments, supplement tissue results to provide a more comprehensive tumor profiling of Met x Met resistance mechanism. (2L, second line; aNSCLC, advanced non-small cell lung cancer; CNV, copy number variation; FMI, Foundation Medicine, Inc .; NGS, next generation sequencing; NSV, non-synonymous variations; SNV, single nucleotide variant.)

FIG. 18 illustrates unbiased clustering of baseline somatic variants with confirmed MET alterations. Several genes with known functional significance were detected, which clustered by cohort assignment, centrally confirmed MET alterations and EGFR status. (Amp, amplification; EGFR, epidermal growth factor receptor; Ex14, exon 14; FMI, Foundation Medicine, Inc .; IHC, immunohistochemistry; MET, N, no; NA, not available; mesenchymal epithelial transition; O/E, overexpression; SNV, single nucleotide variant; TKI, tyrosine kinase inhibitor; Y, yes.)

FIG. 19 illustrates unbiased clustering of baseline somatic variants with MET alterations and clinical response. Several genes with known functional significance were detected, which clustered by cohort assignment, confirmed MET alterations, EGFR status and Clinical Response.

FIG. 20 depicts classification and examples of baseline somatic mutations identified by tumor profiling. These mutations were identified in non-responders and can lead to MET bypass resistance mechanisms and potentially affect clinical response to REGN5093 even in the presence of MET oncogenic drivers. (Amp, amplification; EGFR, epidermal growth factor receptor; Ex14, exon 14; GOF, gain of function; LOF, loss of function; TKI, tyrosine kinase inhibitor)

FIG. 21 graphically represents total REGN5093 and sMET concentrations during study treatment and individual concentrations of sMET over time by best overall response. Total concentration of REGN5093 was several fold higher than total sMET concentration in serum, suggesting saturation of receptor occupancy was achieved with the 2000 mg Q3W dose regimen. sMET and cHGF levels increased post-dose suggesting target engagement, but neither baseline nor post-treatment levels of sMET and cHGF was significantly associated with response (data not shown). (cHGF, circulating hepatocyte growth factor; Q3W, every 3 weeks; SMET, soluble mesenchymal epithelial transition)

FIG. 22 illustrates the lack of significant association between baseline concentrations of sMET or cHGF and clinical response. cHGF, circulating hepatocyte growth factor; PD, progressive disease; PR, partial response; SD, stable disease; sMET, soluble mesenchymal epithelial transition.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc., including 100).

Although any methods and materials similar or equivalent to those described herein can be used in the practice or testing of the present invention, the preferred methods and materials are now described. All patents, applications and non-patent publications mentioned in this specification are incorporated herein by reference in their entireties.

### Met Protein

The expressions “MET,” “c-Met,” and the like, as used herein, refer to the human membrane spanning receptor tyrosine kinase comprising (1) the amino acid sequence as set forth in SEQ ID NO:13, and/or having the amino acid sequence as set forth in NCBI accession No. NM_001127500.2, representing the unprocessed preproprotein of isoform “a”, (2) the amino acid sequence as set forth in SEQ ID NO:14, and/or having the amino acid sequence as set forth in NCBI accession No. NM_000236.2, representing the unprocessed preproprotein of isoform “b”, (3) the amino acid sequence as set forth in SEQ ID NO:15, and/or having the amino acid sequence as set forth in NCBI accession No. NM_001311330.1, representing the unprocessed preproprotein of isoform “c”, and/or (3) the mature protein comprising the cytoplasmic alpha subunit (SEQ ID NO:16) shared by all three isoforms and the transmembrane beta subunit (SEQ ID NO:17, 18, or 19 of isoform a, b and c, respectively). The expression “MET” includes both monomeric and multimeric MET molecules. As used herein, the expression “monomeric human MET” means a MET protein or portion thereof that does not contain or possess any multimerizing domains and that exists under normal conditions as a single MET molecule without a direct physical connection to another MET molecule. An exemplary monomeric MET molecule is the molecule referred to herein as “hMET.mmh” comprising the amino acid sequence of SEQ ID NO: 20 (SEQ ID NO:152 from U.S. Pat.No. 11,142,578 Example 3). As used herein, the expression “dimeric human MET” means a construct comprising two MET molecules connected to one another through a linker, covalent bond, non-covalent bond, or through a multimerizing domain such as an antibody Fc domain. An exemplary dimeric MET molecule is the molecule referred to herein as “hMET.mFc” comprising the amino acid sequence of SEQ ID NO: 21 (SEQ ID NO: 153 from U.S. Pat. No. 11,142,578 Example 3).

All references to proteins, polypeptides and protein fragments herein are intended to refer to the human version of the respective protein, polypeptide or protein fragment unless explicitly specified as being from a non-human species. Thus, the expression “MET” means human MET unless specified as being from a non-human species, e.g., “mouse MET,” “monkey MET,” etc.

As used herein, the expression “cell surface-expressed MET” means one or more MET protein(s), or the extracellular domain thereof, that is/are expressed on the surface of a cell in vitro or in vivo, such that at least a portion of a MET protein is exposed to the extracellular side of the cell membrane and is accessible to an antigen-binding portion of an antibody. A “cell surface-expressed MET” can comprise or consist of a MET protein expressed on the surface of a cell which normally expresses MET protein. Alternatively, “cell surface-expressed MET” can comprise or consist of MET protein expressed on the surface of a cell that normally does not express human MET on its surface but has been artificially engineered to express MET on its surface.

Measures of MET alterations in NSCLC include MET exon 14 mutation: oncogenic driver in NSCLC with recent TKI approvals in 1L (first line); MET gene amplification: major resistance mechanism to EGFR TKI therapy in 2L (second line) +NSCLC; and MET protein overexpression, reported to enrich but not select for treatment response to MET TKI's in NSCLC.

### Therapeutic Methods of Treating NSCLC Cancer

Lung cancer is one of the most commonly diagnosed cancers and is the leading cause of cancer-related mortality worldwide (Siegel et al., CA Cancer J Clin, 66(1):7-30, 2016). Non-small cell lung cancer (NSCLC) accounts for 80% to 85% of all lung cancers and is composed of several histopathological subtypes, the most common of which are adenocarcinoma (40% to 60%) and squamous cell carcinoma (30%) (Dela Cruz et al., Clin Chest Med, 32(4):605-44, 2011). The majority of patients with NSCLC are found to have advanced cancer at the time of diagnosis.

Anti-PD-1 and anti-PD-L1 therapies have changed the standard of care for many patients with NSCLC (Topalian et al., NEJM, 366(26):3443-54, 2012). However, data is emerging that suggests MET-driven NSCLC patients may not experience equivalent benefit to agents that target the PD-1/PD-L1 axis, even with tumors that express high PD-L1 or exhibit high tumor mutation burden (TMB) (Sabari et al., Ann Oncol, 29(10):2085-91, 2018). This is in keeping with data generated in lung cancers harboring EGFR mutations or ALK rearrangements (Garassino et al., Lancet Oncol, 19(4):521-36, 2018) (Lee et al., JAMA Oncol, 4(2):210-16 2018) (Peters et al., J Clin Oncol, 35(24):2781-9, 2017) and indicates that monotherapy with anti-PD-1 or anti-PD-L1 may not be the preferred treatment for patients with MET-driven disease. Thus, there remains a substantial unmet need for therapies that improve response rates and survival for patients with MET-altered NSCLC.

The present inventors have surprisingly discovered that certain MET x MET bispecific antibodies are exceptionally suited for treating and/or inhibiting NSCLC or mitigating metastasis of a NSCLC associated with a MET alteration such as an exon 14 alteration in DNA or a deletion that leads to exon 14 skipping, a MET gene amplification, or MET protein overexpression. REGN5093 is an exemplary human bispecific antibody that binds two epitopes on the MET receptor, resulting in blockade of ligand-dependent and ligand-independent signaling with potential activity in lung cancer. The Examples below illustrate that tumor response to treatment with a MET x MET bispecific antibody is enriched by identifying patients having these MET alterations.

As such, useful according to the methods described herein are MET x MET bispecific antibodies comprising a first antigen-binding domain (also referred to herein as “D1”) which specifically binds a first epitope of human MET, and a second antigen-binding domain (also referred to herein as “D2”) which specifically binds a second epitope of human MET. The simultaneous binding of the two separate MET epitopes by the bispecific antibody results in effective ligand blocking with minimal activation of MET signaling. Such MET x MET bispecific antibodies are described in U.S. Publication No. 2018/0134794, incorporated by reference herein in its entirety.

The MET x MET bispecific antibodies are useful, inter alia, for the treatment, prevention and/or amelioration of NSCLC associated with or mediated by MET expression, signaling or activity, or treatable by blocking the interaction between MET and HGF, or otherwise inhibiting MET activity and/or signaling, and/or promoting receptor internalization and/or decreasing cell surface receptor number. The MET x MET bispecific antibodies are useful, inter alia, for the treatment and/or amelioration of NSCLC in a subject suffering from a tumor harboring a MET alteration such as an exon 14 alteration in DNA or a deletion that leads to exon 14 skipping, a MET gene amplification, or MET protein overexpression. The MET x MET bispecific antibodies are useful, inter alia, for the preventing recurrence or metastasis of NSCLC in a subject suffering from a tumor harboring a MET alteration such as an exon 14 alteration in DNA or a deletion that leads to exon 14 skipping, a MET gene amplification, or MET protein overexpression.

For example, MET x MET bispecific antibodies of the present disclosure are useful for the treatment of tumors that express (or overexpress) MET, e.g., NSCLC having a MET alteration. Illustratively, the MET alteration can be an exon 14 alteration in DNA or a deletion that leads to exon 14 skipping, a MET gene amplification, or MET protein overexpression. In some aspects, the MET alteration is an exon 14 alteration in DNA or a deletion that leads to exon 14 skipping. In some aspects, the MET alteration is a MET gene amplification. In some aspects, the MET alteration is MET protein overexpression.

In some embodiments, the subject suffers from non-squamous NSCLC. In some embodiments, the subject suffers from a NSCLC squamous carcinoma. In some aspects, the NSCLC has metastasized. In some aspects, the subject has NSCLC which has metastasized to the brain. In some aspects, the subject has NSCLC which has metastasized to the liver. In some embodiments the NSCLC is unresectable. Treatment includes reducing NSCLC tumor growth and/or causing regression of an NSCLC in a subject.

Illustrative methods of using the MET x MET bispecific antibodies are provided throughout the present disclosure, and detailed below.

Methods of treating NSCLC in a subject can comprise administering to a subject in need thereof a therapeutic composition comprising a MET x MET bispecific antibody (e.g., a MET x MET bispecific antibody comprising the D1 and D2 components as set forth in Table 1 herein, or an anti-MET antibody selected from the group consisting of onartuzumab, emibetuzumab, telisotuzumab, SAIT301, ARGX-111, Sym015, HuMax-cMet, and CE-355621).

Methods of treating NSCLC, reducing growth of a NSCLC tumor, inhibiting or mitigating invasion and/or metastasis, and/or causing regression of an NSCLC in a subject having a tumor that harbors a MET alteration can comprise administering to a subject in need thereof a bispecific antibody comprising a first antigen-binding domain (D1); and a second antigen-binding domain (D2); wherein D1 specifically binds a first epitope of human MET; and wherein D2 specifically binds a second epitope of human MET.

Methods of treating a subject suffering from a NSCLC tumor harboring a MET alteration can comprise administering to the subject a MET x MET bispecific antibody comprising: a first antigen-binding domain (D1); and a second antigen-binding domain (D2); wherein D1 specifically binds a first epitope of human MET; and wherein D2 specifically binds a second epitope of human MET. In some aspects, the MET alteration is an exon 14 alteration in DNA or a deletion that leads to exon 14 skipping. In some aspects, the subject is MET targeted tyrosine kinase inhibitor (MET-TKI) naïve. In some aspects, the MET alteration is a MET gene amplification. In some aspects, the MET alteration is MET protein expression.

Methods of treating NSCLC, reducing NSCLC tumor growth, and or causing regression of NSCLC in a subject suffering from a tumor harboring a MET alteration can comprise administering to the subject a 250 to 2000 mg dose of a MET x MET bispecific antibody.

Methods of treating or inhibiting the growth of a NSCLC can comprise: (1) selecting a subject with a tumor harboring a MET alteration; and (2) administering to the subject (a) a dose of about 250 mg, 500 mg, 750 mg, 1000 mg, 1500 mg, or 2000 mg of a MET x MET bispecific antibody. In some aspects, the administering of step (2) occurs once every 3 weeks.

Additional methods for treating a tumor comprise: (a) selecting a subject with NSCLC; (b) determining that the tumor exhibits a MET alteration selected from the group consisting of an Exon 14 alteration in DNA or a deletion that leads to exon 14 skipping, MET gene amplification, and/or Met protein overexpression, comprising (i) obtaining a tissue sample and/or a liquid sample from the subject; and (ii) assessing the tissue sample for MET gene amplification using fluorescent in situ hybridization in tumor tissues or by Next Gen Sequencing in tumor tissues and or ctDNA and/or assessing the tissue sample for Met protein overexpression using immunohistochemistry, and/or assessing the liquid sample for an Exon14 mutation using ctDNA; and, if the tumor exhibits a MET alteration, (c) administering one or more doses of a MET x MET bispecific antibody to the subject in need thereof.

Identification of a patient or patient population who will likely be successfully treated with the MET x MET antibody is desirable at least from the patient perspective. Thus, the present inventors have conceived of methods for identifying a candidate for MET x MET anti-tumor therapy, the methods comprising obtaining a tissue sample and/or a fluid sample from a subject having NSCLC; and assessing the tissue sample and/or fluid sample for a MET alteration selected from the group consisting of an Exon 14 alteration in DNA or a deletion that leads to exon 14 skipping, MET gene amplification, and/or Met protein overexpression, wherein presence of at least one Met alteration in the tissue sample or fluid sample identifies the subject as a candidate for anti-tumor therapy, wherein the MET x MET anti-tumor therapy comprises a MET x MET bispecific antibody.

Further methods for treating NSCLC in a subject can comprise: (i) obtaining a liquid sample from the subject and determining the MET amplification in ctDNA from the liquid sample, and (ii) administering a MET x MET bispecific antibody to the subject; wherein steps (i) and (ii) are repeated once every three weeks, and wherein a loss of MET amplification after step (ii) is repeated is indicative of durable response to treatment.

In some aspects, the subject according to any one of the methods provided herein has one or more of (i) histologically confirmed NSCLC; (ii) MET-exon14 gene mutation; (iii) MET gene amplification; (iv) elevated MET protein expression (IHC ≥2+ or H score of >150); (v) MET exon 14 gene mutation and MET TKI experienced; (vi) MET exon 14 gene mutation and MET TKI naïve; (vii) MET gene highly amplified (MET GCN ≥5 and/or MET/CEP7 ratio ≥2 by FISH or MET GCN ≥6 by NGS in tissues or MET fold change ≥2 in ctDNA) and MET TKI naïve; (viii) MET protein highly overexpressed (IHC 3+ or H score of ≥200) and MET TKI naïve; (ix) MET gene highly amplified (MET GCN ≥5 and/or MET/CEP7 ratio ≥2 by FISH or MET GCN ≥6 by NGS in tissues or MET fold change ≥2 in ctDNA), MET protein highly overexpressed (IHC 3+ or H score of ≥200), and MET TKI naïve.

In some aspects, the subject is selected as having one or more of the following: Exon 14 alteration in DNA or a deletion that leads to exon 14 skipping with prior MET TKI experience; Exon 14 alteration in DNA or a deletion that leads to exon 14 skipping (MET TKI naïve) with PD-(L)1 experience; Exon 14 alteration in DNA or a deletion that leads to exon 14 skipping (MET TKI naïve) with prior EGFR inhibitor and no PD-(L1) experience; MET gene amplification and/or MET protein overexpression (MET TKI naïve) and no PD-(L1) experience; MET gene amplification and/or MET protein overexpression (MET TKI naïve) with PD-(L)1 experience; or MET gene amplification and/or MET protein overexpression (MET TKI naïve) with prior EGFR inhibitor and no PD-(L)1 experience. The subject can also be selected on the basis of having a tumor with one or more mutations in the EGFR gene.

In some embodiments, the subject has not received prior anti-cancer therapy. In some embodiments, the subject is MET targeted tyrosine kinase inhibitor (TKI) naïve. In other words, the subject has not received prior treatment with a TKI. In some embodiments, the subject has received prior anti-cancer therapy comprising one or more of a TKI, PD-1 inhibitor, a PD-L1 inhibitor, surgery, radiation therapy, or chemotherapy. In some aspects, the prior anti-cancer therapy comprises a TKI. In some aspects, the subject is resistant or inadequately responsive to, or relapsed after prior therapy.

In the context of the methods of treatment described herein, the MET x MET bispecific antibodies may be administered as a monotherapy (i.e., as the only therapeutic agent) or in combination with one or more additional therapeutic agents.

Responsiveness to treatment with a MET x MET bispecific antibody can change over time as tumor cells develop bypass resistance mechanisms. Resistance to MET therapy can be acquired in response to prior therapies in heavily pre-treated NSCLC patients, for example, patients treated with chemotherapy, immune checkpoint inhibitors, and/or EGFR inhibitors. It is desirable to provide alternative treatments as early as possible if a tumor has proven resistant to a current therapeutic. As such, methods for monitoring efficacy of treatment with a MET x MET bispecific antibody in a subject having NSCLC with a MET alteration are provided herein. The methods comprise: (i) obtaining a tissue sample and/or a fluid sample from the subject and assessing the tissue sample and/or fluid sample for somatic mutations in one or more genes selected from the group consisting of:
a. an on-target MET Receptor gene mutation that confers resistance to MET TKI's found in MET Ex14 Mut patients with prior TKI Exp and MET gene silencing (loss-of-function);
b. TK driver receptor activation selected from TK Receptor and ligand gene amplification and TKR activating mutations; and
c. an activating gene mutation in a pathway selected from the group consisting of JAK2/STAT3 pathway, RAS/RAF/MEK/MAPK pathway, PI3K/AKT/MTOR pathway, TP53 mutations, and cell cycle gene amplification;
   (ii) administering a MET x MET bispecific antibody to the subject; and (iii) repeating steps (i) and (ii) over the course of treatment; wherein the acquiring of a mutation by one or more genes is indicative of resistance to therapy and/or indicative of poor prognosis.

In some aspects, the on-target MET Receptor gene mutation is selected from the group consisting of MET Y1230C, MET D1228H, MET D1228N; and the MET gene silencing (loss-of-function) is selected from somatic mutations in DNMT3A and TET2.

In some aspects, the TKR activating mutation is selected from the group consisting of EGFR L858R, EGFR G719S, EGFR E709A, EGFR E746_A750del, and EGFR S752_I759del.

In some aspects, the JAK2/STAT3 pathway mutation is JAK2 V617F; the RAS/RAF/MEK/MAPK pathway mutation is selected from the group consisting of KRAS G12A/V, GNAS R201H, MKRN-BRAF fusion, BRAF S602Y, RICTOR Amp, and MAP2K1 K57N; the PI3K/AKT/MTOR pathway mutation is selected from the group consisting of PIK3CA H1047L, PIK3CA E545K, PIK3CA E542K, PIK3CA N345K, IDH1 R132L, and MTOR E2338Q; the PI3K/AKT/MTOR pathway amplification is selected from the group consisting of AKT2 Amp and RICTOR Amp; the TP53 mutation is selected from the group consisting of TP53 R280T and TP53 R248Q; and the cell cycle gene amplification is selected from the group consisting of CDK4 Amp, CDK6 Amp, CCND1 Amp, and CCNE1 Amp.

Various aspects of the MET x MET bispecific antibody are provided in the following paragraphs, though described in greater detail elsewhere herein.

In some aspects, D1 and D2 do not compete with one another for binding to human MET. In some aspects, the first epitope of human MET comprises amino acids 192-204 of SEQ ID NO:22. In some aspects, the second epitope of human MET comprises amino acids 305-315 and 421-455 of SEQ ID NO:22. In some aspects, the first epitope of human MET comprises amino acids 192-204 of SEQ ID NO:22; and the second epitope of human MET comprises amino acids 305-315 and 421-455 of SEQ ID NO:22.

In some embodiments, D1 comprises three heavy chain complementarity determining regions (HCDR1, HCDR2 and HCDR3) within a heavy chain variable region (HCVR) comprising the amino acid sequence of SEQ ID NO: 1 and three light chain complementarity determining regions (LCDR1, LCDR2 and LCDR3) within a light chain variable region (LCVR) comprising the amino acid sequence of SEQ ID NO:9. In some embodiments, D2 comprises three heavy chain complementarity determining regions (HCDR1, HCDR2 and HCDR3) within a heavy chain variable region (HCVR) comprising the amino acid sequence of SEQ ID NO: 5 and three light chain complementarity determining regions (LCDR1, LCDR2 and LCDR3) within a light chain variable region (LCVR) comprising the amino acid sequence of SEQ ID NO: 9.

In some embodiments, the bispecific antibody comprises the CDRs within the D1-HCVR amino acid sequence of SEQ ID NO: 1 and the CDRs within the D2-HCVR amino acid sequence of SEQ ID NO: 5.

In some aspects, the bispecific antibody D1 comprises three heavy chain complementarity determining regions (HCDR1, HCDR2 and HCDR3) within a heavy chain variable region (HCVR) comprising the amino acid sequence of SEQ ID NO:1 or an amino acid sequence that is at least 95% identical thereto and three light chain complementarity determining regions (LCDR1, LCDR2 and LCDR3) within a light chain variable region (LCVR) comprising the amino acid sequence of SEQ ID NO:9 or an amino acid sequence that is at least 95% identical thereto.

In some aspects, the D1 HCDR1 comprises the amino acid sequence of SEQ ID NO:2; HCDR2 comprises the amino acid sequence of SEQ ID NO:3; HCDR3 comprises the amino acid sequence of SEQ ID NO:4; LCDR1 comprises the amino acid sequence of SEQ ID NO:10; LCDR2 comprises the amino acid sequence of SEQ ID NO:11; and LCDR3 comprises the amino acid sequence of SEQ ID NO:12.

In some aspects, the bispecific antibody D1 comprises an HCVR comprising the amino acid sequence of SEQ ID NO: 1 or an amino acid sequence that is at least 95% identical thereto; and an LCVR comprising the amino acid sequence of SEQ ID NO: 9 or an amino acid sequence that is at least 95% identical thereto.

In some aspects, the bispecific antibody D1 comprises an HCVR comprising the amino acid sequence of SEQ ID NO: 1; and an LCVR comprising the amino acid sequence of SEQ ID NO: 9.

In some aspects, the bispecific antibody D2 comprises three heavy chain complementarity determining regions (HCDR1, HCDR2 and HCDR3) within a heavy chain variable region (HCVR) comprising the amino acid sequence of SEQ ID NO: 5 or an amino acid sequence that is at least 95% identical thereto and three light chain complementarity determining regions (LCDR1, LCDR2 and LCDR3) within a light chain variable region (LCVR) comprising the amino acid sequence of SEQ ID NO: 9 or an amino acid sequence that is at least 95% identical thereto.

In some aspects, the bispecific antibody D2 HCDR1 comprises the amino acid sequence of SEQ ID NO: 6; HCDR2 comprises the amino acid sequence of SEQ ID NO: 7; HCDR3 comprises the amino acid sequence of SEQ ID NO: 8; LCDR1 comprises the amino acid sequence of SEQ ID NO:10; LCDR2 comprises the amino acid sequence of SEQ ID NO: 11; and LCDR3 comprises the amino acid sequence of SEQ ID NO: 12.

In some aspects, the bispecific antibody D2 comprises an HCVR comprising the amino acid sequence of SEQ ID NO: 5 or an amino acid sequence that is at least 95% identical thereto; and an LCVR comprising the amino acid sequence of SEQ ID NO: 9 or an amino acid sequence that is at least 95% identical thereto.

In some aspects, the bispecific antibody D2 comprises an HCVR comprising the amino acid sequence of SEQ ID NO: 5; and an LCVR comprising the amino acid sequence of SEQ ID NO: 9.

### Biological Characteristics of the Antibodies Provided Herein

Useful according to the methods provided herein are bispecific antibodies and antigen-binding fragments thereof that inhibit proliferation, inhibit invasion, cause apoptosis, and/or decrease viability of an NSCLC cell. The bispecific antibodies and antigen-binding fragments thereof bind MET receptors and prevent interaction with HGF.

Also useful according to the methods provided herein are bispecific antibodies and antigen-binding fragments thereof that bind monomeric human MET with high affinity. For example, the present disclosure includes anti-MET x MET antibodies that bind monomeric human MET (e.g., hMET.mmh) with a K_{D} of less than about 230 nM as measured by surface plasmon resonance at 25° C. or 37° C., e.g., using an assay format as defined in U.S. Pat. No. 11,142,578 Example 3, or a substantially similar assay. According to certain embodiments, anti-MET antibodies are useful according to the methods provided herein bind monomeric human MET at 37° C. with a K_{D} of less than about 230 nM, less than about 200 nM, less than about 150 nM, less than about 100 nM, less than about 50 nM, less than about 25 nM, less than about 20 nM, less than about 10 nM, Less than about 8 nM, less than about 6 nM, less than about 5 nM, less than about 4 nM, or less than about 3 nM, as measured by surface plasmon resonance, e.g., using an assay format as defined in U.S. Pat. No. 11, 142,578, Example 3, or a substantially similar assay.

Such bispecific antibodies and antigen-binding fragments thereof bind monomeric human MET (e.g., hMET.mmh) with a dissociative half-life (t½) of greater than about 1 minute as measured by surface plasmon resonance at 25° C. or 37° C., e.g., using an assay format as defined in U.S. Pat. No. 11,142,578 Example 3, or a substantially similar assay. Such bispecific antibodies are provided that bind monomeric human MET at 37° C. with a t½ of greater than about 1 minute, greater than about 2 minutes, greater than about 4 minutes, greater than about 6 minutes, greater than about 8 minutes, greater than about 10 minutes, greater than about 12 minutes, greater than about 14 minutes, greater than about 16 minutes, greater than about 18 minutes, or greater than about 20 minutes, or longer, as measured by surface plasmon resonance, e.g., using an assay format as defined in U.S. Pat. No. 11,142,578, Example 3, or a substantially similar assay.

Such bispecific antibodies and antigen-binding fragments thereof bind dimeric human MET (e.g., hMET.mFc) with high affinity. For example, the bispecific antibodies bind dimeric human MET with a K_{D} of less than about 3 nM as measured by surface plasmon resonance at 25° C. or 37° C., e.g., using an assay format as defined in U.S. Pat. No. 11,142,578 Example 3, or a substantially similar assay. According to certain embodiments, anti-MET antibodies are provided that bind dimeric human MET at 37° C. with a K_{D} of less than about 3 nM, less than about 2 nM, less than about 1 nM, less than about 0.9 nM, less than about 0.8 nM, less than about 0.7 nM, less than about 0.6 nM, less than about 0.5 nM, less than about 0.4 nM, less than about 0.3 nM, or less than about 0.25 nM, as measured by surface plasmon resonance, e.g., using an assay format as defined in U.S. Pat. No. 11, 142,578, Example 3, or a substantially similar assay.

Such bispecific antient binding molecules and antigen-binding fragments thereof may bind dimeric human MET (e.g., hMET.mFc) with a dissociative half-life (t½) of greater than about 4 minutes as measured by surface plasmon resonance at 25° C. or 37° C., e.g., using an assay format as defined in U.S. Pat. No. 11,142,578 Example 3, or a substantially similar assay. According to certain embodiments, anti-MET antibodies are provided that bind dimeric human MET at 37° C. with a t½ of greater than about 4 minutes, greater than about 5 minutes, greater than about 10 minutes, greater than about 20 minutes, greater than about 30 minutes, greater than about 40 minutes, greater than about 50 minutes, greater than about 60 minutes, greater than about 70 minutes, greater than about 80 minutes, greater than about 90 minutes, greater than about 100 minutes, greater than about 105 minutes, or longer, as measured by surface plasmon resonance, e.g., using an assay format as defined in U.S. Patent No. 11,142,578, Example 3, or a substantially similar assay.

Also useful according to the methods provided herein are MET x MET bispecific antigen-binding proteins that bind dimeric human MET (e.g., hMET.mFc) with a dissociative half-life (t½) of greater than about 10 minutes as measured by surface plasmon resonance at 25° C. or 37° C., e.g., using an assay format as defined in U.S. Pat. No. 11, 142,578, Example 6, or a substantially similar assay. According to certain embodiments, MET x MET bispecific antigen-binding proteins are provided that bind dimeric human MET at 37° C. with a t½ of greater than about 10 minutes, greater than about 20 minutes, greater than about 30 minutes, greater than about 40 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, greater than about 1100 minutes, or longer, as measured by surface plasmon resonance, e.g., using an assay format as defined in U.S. Pat. No. 11,142,578, Example 6, or a substantially similar assay.

Also useful according to the methods provided herein are MET x MET bispecific antibodies that block the interaction between HGF and MET, e.g., in an in vitro ligand-binding assay. According to certain embodiments provided herein, the MET x MET bispecific antigen-binding proteins block HGF binding to cells expressing human MET, and induce minimal or no MET activation in the absence of HGF signaling. For example, the present disclosure provides MET x MET bispecific antigen-binding proteins that exhibit a degree of MET agonist activity in a cell-based MET activity reporter assay that is less than 50%, less than 40%, less than 30%, less than 20%, less than 10%, less than 5%, less than 3%, less than 2% or less than 1% of the MET agonist activity observed in an equivalent activity reporter assay using a monospecific antibody comprising D1 or D2 alone.

The bispecific antibodies useful according to the present disclosure may possess one or more of the aforementioned biological characteristics, or any combination thereof. The foregoing list of biological characteristics of the antibodies is not intended to be exhaustive. Other biological characteristics of the antibodies provided herein will be evident to a person of ordinary skill in the art from a review of the present disclosure including the working Examples herein.

### Epitope Mapping and Related Technologies

Useful according to the methods provided herein are MET x MET bispecific antibodies that bind a human MET epitope comprising amino acids 192-204, amino acids 305-315, and/or amino acids 421-455 of SEQ ID NO:22.

### Preparation of Human Antibodies

The MET x MET bispecific antibodies useful according to the methods provided herein can be fully human antibodies. Methods for generating monoclonal antibodies, including fully human monoclonal antibodies are known in the art. Any such known methods can be used in the context of the present disclosure to make human antibodies that specifically bind to human MET.

Using VELOCIMMUNE™ technology, for example, or any other similar known method for generating fully human monoclonal antibodies, high affinity chimeric antibodies to MET are initially isolated having a human variable region and a mouse constant region. As in the experimental section below, the antibodies are characterized and selected for desirable characteristics, including affinity, ligand blocking activity, selectivity, epitope, etc. If necessary, mouse constant regions are replaced with a desired human constant region, for example wild-type or modified lgG1 or lgG4, to generate a fully human anti-MET antibody. While the constant region selected may vary according to specific use, high affinity antigen-binding and target specificity characteristics reside in the variable region. In certain instances, fully human anti-MET antibodies are isolated directly from antigen-positive B cells.

### Bioequivalents

The methods described herein can utilize MET x MET bispecific antibodies and antibody fragments thereof that encompass proteins having amino acid sequences that vary from those of the described antibodies but that retain the ability to bind human MET. Such variant molecules and antibody fragments comprise one or more additions, deletions, or substitutions of amino acids when compared to parent sequence but exhibit biological activity that is essentially equivalent to that of the described molecules. Likewise, the anti-MET x MET antibody-encoding DNA sequences of the present disclosure encompass sequences that comprise one or more additions, deletions, or substitutions of nucleotides when compared to the disclosed sequence, but that encode an anti-MET x MET antibody or antibody fragment that is essentially bioequivalent to an anti-MET antibody or antibody fragment of the disclosure. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a subject can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-MET antibodies provided herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-MET antibody variants comprising amino acid changes which modify the glycosylation characteristics of the antibodies, e.g., mutations which eliminate or remove glycosylation.

### Species Selectivity and Species Cross-Reactivity

The present disclosure, according to certain embodiments, provides methods of using anti-MET antibodies (and antibodies comprising anti-MET antigen-binding domains) that bind to human MET but not to MET from other species. Also useful are anti-MET antibodies (and antibodies comprising anti-MET antigen-binding domains) that bind to human MET and to MET from one or more non-human species. For example, the anti-MET x MET antibodies and antibodies may bind to human MET and may bind or not bind, as the case may be, to one or more of mouse, rat, guinea pig, hamster, gerbil, pig, cat, dog, rabbit, goat, sheep, cow, horse, camel, cynomolgus, marmoset, rhesus or chimpanzee MET. According to certain exemplary embodiments, anti-MET x MET antibodies and antibodies are provided which specifically bind human MET and cynomolgus monkey (e.g., Macaca fascicularis) MET. Other anti-MET x MET antibodies and antibodies bind human MET but do not bind, or bind only weakly, to cynomolgus monkey MET.

### Therapeutic Formulation and Administration

Useful herein are pharmaceutical compositions comprising the MET x MET bispecific antibodies of the present invention. The pharmaceutical compositions may be formulated with suitable carriers, excipients, and other agents that provide improved transfer, delivery, tolerance, and the like.

In some aspects, the pharmaceutical compositions comprising the MET x MET bispecific antibodies are formulated for administration to a subject for treating lung cancer, and specifically, for treating NSCLC.

Provided herein are methods in which the MET x MET bispecific antibodies that are administered to the subject are contained within a pharmaceutical formulation. The pharmaceutical formulation may comprise the MET x MET bispecific antibody along with at least one inactive ingredient such as, e.g., a pharmaceutically acceptable carrier. Other agents may be incorporated into the pharmaceutical composition to provide improved transfer, delivery, tolerance, and the like. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the antibody is administered. A multitude of appropriate formulations can be found in the formulary known to all pharmaceutical chemists: Remington's Pharmaceutical Sciences (15th ed, Mack Publishing Company, Easton, Pa., 1975), particularly Chapter 87 by Blaug, Seymour, therein. These formulations include, for example, powders, pastes, ointments, jellies, waxes, oils, lipids, lipid (cationic or anionic) containing vesicles (such as LIPOFECTIN.TM.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in the context of the methods of the present disclosure, provided that the anti-MET antibody or MET x MET bispecific antibody is not inactivated by the formulation and the formulation is physiologically compatible and tolerable with the route of administration. See also Powell et al. PDA (1998) J Pharm Sci Technol. 52:238-311 and the citations therein for additional information related to excipients and carriers well known to pharmaceutical chemists.

Pharmaceutical formulations useful for administration by injection in the context of the present disclosure may be prepared by dissolving, suspending or emulsifying an anti-MET antibody or MET x MET bispecific antibody in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there may be employed, e.g., sesame oil, soybean oil, etc., which may be used in combination with a solubilizing agent such as benzyl benzoate, benzyl alcohol, etc. The injection thus prepared can be filled in an appropriate ampoule if desired.

### Administration Regimens

According to certain embodiments, multiple doses of an anti-MET antibody or MET x MET bispecific antibody (or a pharmaceutical composition comprising a combination of an anti-MET antibody or MET x MET bispecific antibody and any of the additional therapeutically active agents mentioned herein) may be administered to a subject over a defined time course. The methods according to this aspect comprise sequentially administering to a subject multiple doses of an anti-MET antibody or MET x MET bispecific antibody provided herein. As used herein, “sequentially administering” means that each dose of antibody is administered to the subject at a different point in time, e.g., on different days separated by a predetermined interval (e.g., hours, days, weeks or months). The present disclosure includes methods which comprise sequentially administering to the subject a single initial dose of an anti-MET antibody or MET x MET bispecific antigen-binding molecule, followed by one or more secondary doses of the anti-MET antibody or MET x MET bispecific antigen-binding molecule, and optionally followed by one or more tertiary doses of the anti-MET antibody or MET x MET bispecific antibody.

The terms “initial dose,” “secondary doses,” and “tertiary doses,” refer to the temporal sequence of administration of the anti-MET antibody or MET x MET bispecific antibody. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-MET antibody or MET x MET bispecific antigen-binding molecule, but generally may differ from one another in terms of frequency of administration. In certain embodiments, however, the amount of antibody contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

The anti-MET antibody or MET x MET bispecific antibody can be administered according to any regimen which provides a therapeutic effect. In some aspects, the bispecific antibody is administered at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some aspects, the bispecific antibody is administered once a week, once every two weeks, once every three weeks, or once a month. In some aspects, the bispecific antibody is administered once every three weeks.

In some aspects, the bispecific antibody is administered one week, two weeks, three weeks, or four weeks after the immediately preceding dose. In some aspects, the bispecific antibody is administered three weeks after the immediately preceding dose.

According to certain embodiments of the present disclosure, multiple doses of bispecific antibody may be administered to a subject over a defined time course. The methods according to this aspect of the disclosure comprise sequentially administering to a subject more than one dose of the bispecific antibody. As used herein, “sequentially administering” means that each dose of the antibody is administered to the subject at a different point in time, e.g., on different days separated by a predetermined interval (e.g., hours, days, weeks or months). The present disclosure includes methods which comprise sequentially administering to the subject a single initial dose of a bispecific antigen-binding molecule, followed by one or more secondary doses of the bispecific antigen-binding molecule, and optionally followed by one or more tertiary doses of the bispecific antibody.

According to certain embodiments of the present disclosure, multiple doses of a bispecific antibody can be administered to a subject for several months or years, once every 3 or 6 weeks.

The terms “initial dose,” “secondary doses,” and “tertiary doses,” refer to the temporal sequence of administration. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the antibody (anti-MET x MET bispecific antigen-binding molecule). In certain embodiments, however, the amount contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). For example, a bispecific antibody may be administered to a subject with NSCLC at a loading dose of about 1000-3000 mg followed by one or more maintenance doses of about 500 mg, 1000 mg, or 2000 mg.

In one exemplary embodiment of the present disclosure, each secondary and/or tertiary dose is administered ½ to 14 (e.g., ½, 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of the bispecific antibody which is administered to a patient prior to the administration of the very next dose in the sequence with no intervening doses.

The methods according some aspects may comprise administering to a subject any number of secondary and/or tertiary doses of the bispecific antibody. For example, in certain embodiments, only a single secondary dose is administered to the subject. In other embodiments, two or more (e.g., 2, 3, 4, 5, 6, 7, 8, or more) secondary doses are administered to the subject. Likewise, in certain embodiments, only a single tertiary dose is administered to the subject. In other embodiments, two or more (e.g., 2, 3, 4, 5, 6, 7, 8, or more) tertiary doses are administered to the subject.

In embodiments involving multiple secondary doses, each secondary dose may be administered at the same frequency as the other secondary doses. For example, each secondary dose may be administered to the subject 1 to 2 weeks after the immediately preceding dose. Similarly, in embodiments involving multiple tertiary doses, each tertiary dose may be administered at the same frequency as the other tertiary doses. For example, each tertiary dose may be administered to the subject 2, 3, or 4 weeks after the immediately preceding dose. Alternatively, the frequency at which the secondary and/or tertiary doses are administered to a subject can vary over the course of the treatment regimen. The frequency of administration may also be adjusted during the course of treatment by a physician depending on the needs of the individual subject following clinical examination.

In certain embodiments, one or more doses of the bispecific antibody are administered at the beginning of a treatment regimen as “induction doses” on a more frequent basis (twice a week, once a week or once in 2 weeks) followed by subsequent doses (“consolidation doses” or “maintenance doses”) that are administered on a less frequent basis (e.g., once in 4-12 weeks).

### Dosage

The amount of the MET x MET bispecific antibody administered to a subject according to the methods of the present disclosure is, generally, a therapeutically effective amount. As used herein, the phrase “therapeutically effective amount” means an amount of the bispecific antibody that results in, or has the therapeutic effect of, one or more of: (a) a reduction in the severity or duration of a symptom of cancer; (b) inhibition of tumor growth, or an increase in tumor necrosis, tumor shrinkage and/or tumor disappearance; (c) delay in tumor growth and development; (d) inhibit or retard or stop tumor metastasis; (e) prevention of recurrence of tumor growth; (f) increase in survival of a subject with a cancer; and/or (g) a reduction in the use or need for conventional anti-cancer therapy (e.g., reduced or eliminated use of chemotherapeutic or cytotoxic agents) as compared to an untreated subject.

In some instances, a therapeutically effective amount can be from about 250 mg to about 8000 mg, e.g., about 250 mg, about 300 mg, about 350 mg, about 400 mg, about 450 mg, about 500 mg, about 550 mg, about 600 mg, about 700 mg, about 800 mg, about 900 mg, about 1000 mg, about 1050 mg, about 1100 mg, about 1500 mg, about 1600 mg, about 1700 mg, about 2000 mg, about 2050 mg, about 2100 mg, about 2200 mg, about 2500 mg, about 2700 mg, about 2800 mg, about 2900 mg, about 3000 mg, about 3200 mg, about 4000 mg, about 5000 mg, about 6000 mg, about 7000 mg, or about 8000 mg of the MET x MET bispecific antibody.

In some aspects, the bispecific antibody can be administered at a dose of about 250 mg to about 5000 mg, or a dose of about 250 mg, 300 mg, 400 mg, 500 mg, 600 mg, 700 mg, 750 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, 2100 mg, 2200 mg, 2300 mg, 2400 mg, 2500 mg, 3000 mg, 3500 mg, 4000 mg, 4500 mg, or 5000 mg. In some aspects, the dose is about 500 mg MET x MET bispecific antibody. In some aspects, the dose is about 1000 mg MET x MET bispecific antibody. In some aspects, the bispecific antibody is administered at a dose of about 2000 mg. In some aspects, the MET X MET bispecific antibody can be administered at a dose of 250 mg, 500 mg, 1000 mg, or 2000 mg.

The bispecific antibody can be administered intravenously, subcutaneously, or intraperitoneally. In some aspects, the bispecific antibody is administered by intravenous infusion.

### EXAMPLES

The following examples are put forth so as to provide those of ordinary skill in the art with a complete disclosure and description of how to make and use the methods and compositions provided herein and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Construction of Bispecific Antibodies Having Two Different Antigen-Binding Domains Specific for Different Epitopes of MET

U.S. Pat. No. 11,142,578 (incorporated by reference herein in its entirety), Examples 1 through 3, describes the construction of a bispecific antibody comprising two different antigen-binding domains derived from two bivalent, monospecific anti-MET antibodies (a “D1” arm derived from the exemplary anti-MET antibody H4H13306P2 and a “D2” arm derived from the exemplary anti-MET antibody H4H13312P2) and, consequently, bind to separate epitopes on the MET extracellular domain:

Binding epitope of Anti-Met antibody H4H13312P2: AA 192-204: VRRLKETKDGFMF (SEQ ID NO: 23) of SEQ ID NO: 22.

Binding epitope of Anti-Met antibody H4H13306P2: AA 305-315: LARQIGASLND (SEQ ID NO: 24) of SEQ ID NO: 22 and AA 421-455:

(SEQ ID NO: 25)
FIKGDLTIANLGTSEGRFMQVVVSRSGPSTPHVNF of SEQ ID
NO: 22.

Both antigen-binding domains (D1 and D2) comprise a common light chain variable region. The components of the bispecific antibody useful according to the methods provided herein are summarized in Table 1.

TABLE 1
MET × MET Bispecific Antibody Components Summary

| | SEQ ID NOs: (Amino Acid Sequences) | | | | | | | |
|---|---|---|---|---|---|---|---|---|
| | First Antigen-Binding | | | | Second Antigen-Binding | | | |
| | Domain (D1) H4H13306P2 | | | | Domain (D2) H4H13312P2 | | | |
| | D1- | D1- | D1- | D1- | D2- | D2- | D2- | D2- |
| | HCVR | HCDR1 | HCDR2 | HCDR3 | HCVR | HCDR1 | HCDR2 | HCDR3 |
| H4H14639D | 1 | 2 | 3 | 4 | 5 | 6 | 7 | 8 |
| Bispecific | | | | | | | | |
| Antibody; | | | | | | | | |
| aka | | | | | | | | |
| REGN5093 | | | | | | | | |
| | D1- | D1- | D1- | D1- | D2- | D2- | D2- | D2- |
| | LCVR | LCDR1 | LCDR2 | LCDR3 | LCVR | LCDR1 | LCDR2 | LCDR3 |
| | 9 | 10 | 11 | 12 | 9 | 10 | 11 | 12 |

Binding kinetic parameters for H4H14639D to monomeric Met protein (hMET.mmh) are shown in Table 2.

TABLE 2
Biacore Binding Affinity of H4H14639D at 37° C.
Binding at 37° C./Antibody-Capture Format

| Bispecific | | ka | kd | K_{D} | T½ |
|---|---|---|---|---|---|
| Antibody | Analyte | (Ms⁻¹) | (s⁻¹) | (Molar) | (min) |
| H4H14639D | hMet.mmh | N/A | 1.63E−04 | N/A | 70.8 |

H4H14639D exhibits a dissociation rate which is significantly lower than the dissociation rates of each of its parental antibodies, H4H13306P2 and H4H13312P2. See Table 3.

TABLE 3
Biacore Binding Affinities of Bispecific Anti-MET mAb
and Monospecific Parents at 37° C.
Binding at 37° C./Antibody-Capture Format

| Antibody | Analyte | kd (s⁻¹) | T½ (min) |
|---|---|---|---|
| H4H13306P2 | hMet.mmh | 1.66E−02 | 0.7 |
| H4H13312P2 | hMet.mmh | 8.40E−03 | 1.4 |
| H4H14639D | hMet.mmh | 1.63E−04 | 70.8 |

As noted in U.S. Pat. No. 11,142,578, Example 7, the MET x MET bispecific antibody blocks HGF signaling and exhibits low MET agonist activity.

### Example 2. Anti-Met Antibodies Inhibit Growth of Met-Amplified Cells

The blocking activity of a MET x MET bispecific antibody (i.e., H4H14639D) was assessed in the non-small cell lung cancer (NSCLC) cell line EBC-1, which exhibits amplified Met gene and overexpresses MET (Lutterbach et al., Cancer Res. 67(5): 2081-2088, 2007). Complete growth media for the EBC-1 cells contained MEM Earle's Salts, 10% fetal bovine serum (FBS), penicillin/streptomycin/glutamine, and non-essential amino acids for MEM. H4H14369D exhibited the greatest percent inhibition in MET activity according to the SRE-Luciferase read-out. In the current experiment, 3.0×10³ EBC-1 cells were seeded in complete growth media in the presence of H4H14639D at concentrations ranging from 15 pM to 100 nM. Cells were incubated for 3 days at 37° C. in 5% CO₂. The cells were then fixed in 4% formaldehyde and stained with 3 μg/ml Hoechst 33342 to label the nuclei. Images were acquired on the IMAGEXPRESS® Micro XL (Molecular Devices, Sunnyvale, CA) and nuclear counts were determined via METAXPRESS® Image Analysis software (Molecular Devices, Sunnyvale, CA). Background nuclear counts from cells treated with 40 nM digitonin were subtracted from all wells and viability was expressed as a percentage of the untreated controls. IC50 values were determined from a four-parameter logistic equation over a 10-point response curve (GRAPHPAD PRISM®). As summarized in Table 4, below, the MET x MET bispecific antibody H4H14639D inhibited growth of EBC-1 cells by 37, and with an IC50 of 0.82 nM.

TABLE 4
Anti-Met Bispecific Antibody Blocks EBC-1

| | | % Growth |
|---|---|---|
| | IC₅₀ (nM) | Inhibition |
| mAb | EBC-1 | EBC-1 |
| H4H14639D | 0.82 | 37 |

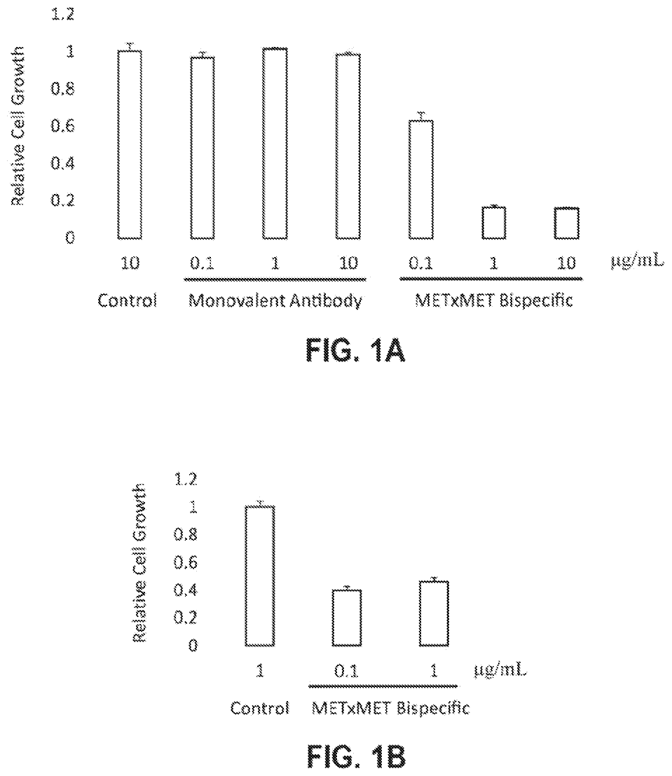

The effect of MET x MET bispecific antibody on the growth of EBC-1 cells was assessed. 2,500 EBC-1 cells were seeded in a 96 well plate and cultured in Dulbecco's Media supplemented with 10% FBS. The cells were treated with a control antibody or a MET x MET bispecific antibody at 0.1 μg/mL or 1 μg/mL, and were subsequently incubated with 5% CO₂ at 37° C. After 5 days, relative cell growth was determined by measuring the reduction of the indicator dye ALAMARBLUE® to its highly fluorescent form in a SPECTRAMAX® M3 plate reader (Molecular Devices, LLC, Sunnyvale, CA). The results are shown in Table 5 and FIG. 1. The MET x MET bispecific antibody (H4H14639D) significantly reduced the relative cell growth of EBC-1 cells compared to the control antibody (FIG. 1).

Several anti-MET antibodies, both bivalent monospecific and MET x MET bivalent, are potent inhibitors of SRE-Luc activation and inhibit the growth of Met-amplified and MET-overexpressing cell lines.

TABLE 5
Anti-Met Bispecific Antibody Blocks EBC-1 Cell Growth

| | Relative Cell | |
|---|---|---|
| | Growth (n = 3) | Standard Deviation |
| Control | 1.000 | 0.045 |
| 0.1 μg/mL H4H14639D | 0.397 | 0.032 |
| 1 μg/mL H4H14639D | 0.462 | 0.028 |

### Example 3. A MET x MET Bispecific Antibody Induces Modest and Transient MET Pathway Activity in NCI-H596 NSCLC Cells

The effect of a MET x MET bispecific antibody on the MET pathway in human lung adenosquamous carcinoma cells was assessed in vitro.

250,000 NCI-H596 cells were seeded in a 12 well plate and cultured in RPMI Media supplemented with 10% FBS. The cells were treated with hepatocyte growth factor (HGF) at 50 ng/ml or the MET x MET bispecific antibody H4H14639D at 10 ug/ml in duplicate. The cells were subsequently incubated in 5% CO₂ at 37° C. After 0, 2, 6 or 18 hours, cell lysates were prepared, protein content was normalized and immunoblot analysis was performed. MET phosphorylation and ERK phosphorylation were quantified with the ImageJ image processing program (T. Collins, BioTechniques 43: S25-S30, 2007). Phosphorylation levels were normalized to the Tubulin loading control and are expressed as fold change relative to control treatment. The results are summarized in Table 6.

TABLE 6
Phosphorylation of MET and ERK

| | Phospho-MET | Phospho-ERK |
|---|---|---|
| Treatment (hours) | (mean ± SD) | (mean ± SD) |
| Control (hFc) (18) | 1.0 ± 0.5 | 1.0 ± 0.3 |
| HGF (2) | 202.3 ± 38.7 | 16.7 ± 1.6 |
| HGF (6) | 38.9 ± 4.9 | 12.4 ± 3.9 |
| HGF (18) | 59.2 ± 24.4 | 12.4 ± 0.9 |
| H4H14639D (2) | 69.7 ± 7.0 | 2.2 ± 0.9 |
| H4H14639D (6) | 9.9 ± 7.4 | 0.3 ± 0.4 |
| H4H14639D (18) | 1.4 ± 0.1 | 0.1 ± 0.1 |

HGF treatment of NCI-H596 cells induced strong activation of MET and ERK that peaked at 2 hours and was sustained after 18 hours. Modest MET and ERK phosphorylation was detected with the H4H14636D bispecific antibody treatment, which returned to baseline levels by 18 or 6 hours, respectively.

### Example 4. A MET x MET Bispecific Antibody Induces MET Degradation More Potently Than Monospecific Antibodies in NCI-H596 Lung Cancer Cells

The effect of a MET x MET bispecific antibody and the parental bivalent monospecific anti-MET antibodies on the expression levels of hepatocyte growth factor receptor (HGFR or MET) on human lung adenosquamous carcinoma cells was assessed. 250,000 NCl-H596 human lung adenosquamous carcinoma cells were seeded in a 12-well plate and cultured in RPMI Media supplemented with 10% FBS. The cells were treated with (1) 5 μg/ml of the hFc control molecule, (2) 5 μg/ml of the parental bivalent monospecific anti-MET antibody H4H13306P2, (3) 5 μg/ml of the parental bivalent monospecific anti-MET antibody H4H13312P2, (4) the combination of 2.5 μg/mL of H4H13306P2 and 2.5 μg/mL of H4H13312P2, or (5) 5 μg/ml of the MET x MET bispecific antibody H4H14639D. The cells were subsequently incubated with 5% CO₂ at 37° C. After 18 hours, cell lysates were prepared, protein content was normalized and immunoblot analysis was performed. MET expression was quantified with the ImageJ image processing program (T. Collins, BioTechniques 43: S25-S30, 2007). The results are summarized in Table 7.

TABLE 7
Relative Level of MET Protein

| Molecule | Relative MET Level |
|---|---|
| Control (hFc) | 1 ± 0.03 |
| H4H13306P2 | 0.50 ± 0.01 |
| H4H13312P2 | 0.35 ± 0.04 |
| H4H13306P2 + H4H13312P2 | 0.61 ± 0.04 |
| H4H14639D | 0.24 ± 0.01 |

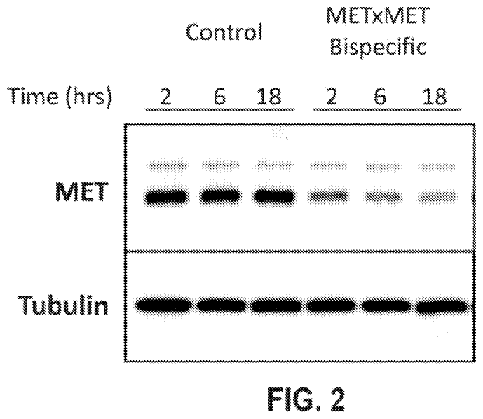

NCI-H596 (MET exon14 skip mutation) lung cancer cells were also treated with control or MET x MET bispecific antibodies at 10 μg/ml for 2, 6 or 18 hrs. MET expression was determined by immunoblotting (FIG. 2), which shows the MET x MET bispecific antibody-induced degradation of MET with increasing time of treatment.

The bispecific antibody, H4H14636D, induces MET degradation more potently than its parental conventional antibodies in NCI-H596 lung cancer cells.

### Example 5. A MET x MET Bispecific Antibody Induces MET Degradation, Inhibits Pathway Activity, and Inhibits Tumor Growth More Potently Than Monospecific Antibodies in EBC-1 Cells

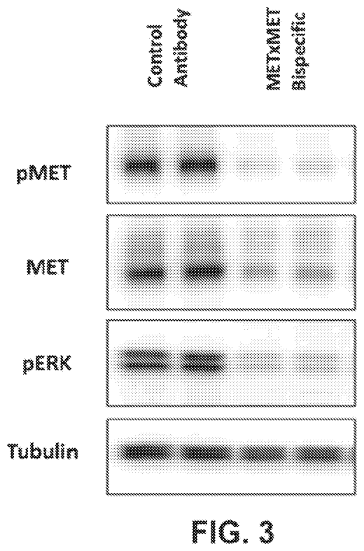

MET-amplified human lung squamous cell carcinoma EBC-1 cells (Lutterbach et al., “Lung cancer cell lines harboring MET gene amplification are dependent on Met for growth and survival,” Cancer Res. 2007 Mar 1;67(5):2081-8) were treated with a control antibody or 10 μg/ml of a MET x MET bispecific antibody for 18 hrs as described above. MET expression and MET pathway activation ascertained by pMET and pErk expression were determined by immunoblotting with the indicated antibodies. The immunoblots are shown in FIG. 3.

Treatment of EBC-1 cells, which harbor MET gene amplification, with MET x MET bispecific antibodies induced more potent degradation of MET than treatment with the control antibody. Treatment of EBC-1 cells with the MET x MET bispecific antibody inhibited downstream effectors of the MET pathway.

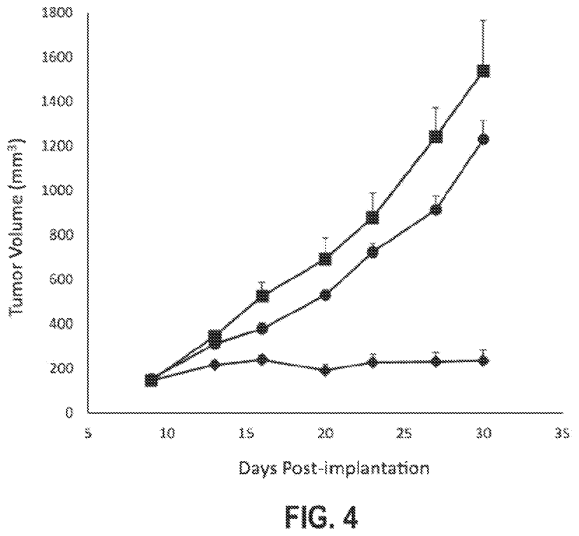

In another experiment, 5 million EBC-1 cells were implanted subcutaneously into the flank of C.B .- 17 SCID mice. Once the tumor volumes reached approximately 150 mm3, mice were randomized into groups of 6 and were treated twice a week with a control antibody at 25 mg/kg or the MET x MET bispecific antibody H4H14639D at 25 mg/kg. Tumor growth was monitored for 30 days post-implantation and tumor volume (mm3) was measured for each experimental group over time. The results are depicted in Table 8 and FIG. 4, which shows that the MET x MET bispecific antibody significantly inhibits the growth of EBC-1 tumors.

TABLE 8
Relative EBC-1 Tumor Growth

| | Tumor Growth (mm3) form the start of treatment |
|---|---|
| Treatment | (mean ± SEM) |
| 25 mg/kg Control | 1394 ± 226 |
| 25 mg/kg H4H14639D | 89 ± 47 |

### Example 6. A MET x MET Bispecific Antibody Does Not Induce Growth of NCI-H596 Lung Cancer Cells in vitro

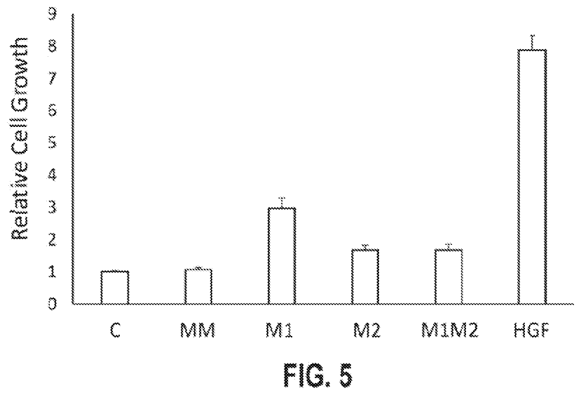

The effect of a MET x MET bispecific antibody on the growth of human non-small cell lung cancer (NSCLC) cells (NCI-H596) was assessed in vitro. 10,000 NCI-H596 lung adenosquamous carcinoma cells (Nair et al., J. Nat'l. Cancer Inst. 86(5): 378-383, 1994) were seeded in 96 well plates on a layer of 0.66% agar in media supplemented with 1% fetal bovine serum (FBS). The cells were cultured in RPMI 1640 media supplemented with 1% FBS with 0.3% agarose. The cells were treated with (1) individual parental bivalent monospecific anti-MET antibodies (H4H13306P2 or H4H13312P2) at 5 μg/ml, (2) a combination of the two parental bivalent monospecific anti-MET antibodies (H4H13306P2 and H4H13312P2) at 2.5 μg/ml each, (3) a bispecific antibody containing one binding arm from H4H13306P2 and the other binding arm from H4H13312P2 (H4H14639D) at 5 μg/ml, or (4) 100 ng/mL of hepatocyte growth factor (HGF). The cells were subsequently incubated with 5% CO₂ at 37° C. After two weeks, relative cell growth was determined by measuring the reduction of the indicator dye, ALAMAR BLUE® (Thermo Fischer Scientific, Waltham, MA), to its highly fluorescent form in a SPECTRAMAX® M3 plate reader (Molecular Devices, Sunnyvale, CA). Increasing fluorescence correlates with cell growth. Table 9 and FIG. 5 depict the relative NCI-H596 cell growth for each antibody treatment normalized to control (no treatment) NCI-H596 cell growth. Treatment of NCI-H596 lung cancer cells with HGF resulted in potent induction of growth in soft agar. The MET x MET (MM in FIG. 5) bispecific antibody H4H14639D did not significantly alter growth relative to control treated cells. Modest induction of cell growth was observed with each parental bivalent monospecific antibody H4H13306P2 (M1) or H4H13312P2 (M2) individually, or combined (H4H13306P2 and H4H13312P2) (M1M2).

TABLE 9
Normalized NCI-H596 Cell Growth

| | Relative Cell | |
|---|---|---|
| | Growth (n = 3) | Standard Deviation |
| Control | 1 | 0.030074808 |
| H4H14639D | 1.070339237 | 0.075103746 |
| H4H13306P2 | 2.9593578 | 0.337877264 |
| H4H13312P2 | 1.686580346 | 0.145670753 |
| H4H13306P2 + H4H13312P2 | 1.693724668 | 0.168651046 |
| HGF | 7.87655937 | 0.46057617 |

### Example 7. Clinical Trial of Met x Met Bispecific Antibody in Non-Small Cell Lung Cancer

### Background:

Lung cancer is one of the most commonly diagnosed cancers and is the leading cause of cancer-related mortality worldwide (Siegel et al., CA Cancer J Clin, 66(1):7-30, 2016). Non-small cell lung cancer (NSCLC) accounts for 80% to 85% of all lung cancers and is composed of several histopathological subtypes, the most common of which are adenocarcinoma (40% to 60%) and squamous cell carcinoma (30%) (Dela Cruz et al., Clin Chest Med, 32(4):605-44, 2011). The majority of patients with NSCLC are found to have advanced cancer at the time of diagnosis.

First-line treatment of advanced NSCLC is guided by the presence of molecular alterations. Patients may receive targeted, small molecule tyrosine kinase inhibitors (TKIs) (Besse et al., Ann Oncol, 25(8):1475-84, 2014) (Ettinger et al., J Natl Compr Canc Netw 15(4):504-35, 2017) (Reck et al., Ann Oncol, 25 Suppl 3:27-39, 2014), immune-checkpoint-inhibitor antibodies that inhibit the PD-1 receptor or PD-1 ligand (PD-L1), or platinum-based doublet chemotherapy regimens, with or without maintenance therapy (Besse et al., Ann Oncol, 25(8):1475-84, 2014) (Ettinger et al., J Natl Compr Canc Netw 15(4):504-35, 2017) (Reck et al., Ann Oncol, 25 Suppl 3:27-39, 2014). Long-term survival remains an unmet need in advanced NSCLC. Overall survival (OS) is approximately 12 months for PD-1/PD-L1 inhibitors, with some studies in TKI-treated patients exceeding 2 years (Camidge et al., J Clin Oncol, 32:abstract 8001, 2014).

Mesenchymal epithelial transition factor (MET) is a single-pass transmembrane tyrosine kinase receptor for the hepatocyte growth factor (HGF). It is expressed in normal tissues such as liver, breast, and adipose and is upregulated in several cancers. High levels of MET expression can occur through increased protein expression or through gene amplification in NSCLC tumors and gastric tumors, and are associated with negative outcomes in patients (Catenacci et al., Cancer, 123(6):1061-70, 2017) (Topalian et al., NEJM, 366(26):2443-54, 2012) (Zhang et al., Hum Pathol, 72:59-65, 2018). Mutations in MET resulting in exon 14 deletions promote prolonged ligand-dependent signaling, resulting in receptor stability and increased tumorigenicity (Kong-Beltran et al., Cancer Res, 66(1): 283-9, 2006).

Approximately 3% of NSCLC is reported to contain MET exon 14 alterations (Cancer Genome Atlas Research Network, Nature, 511(7511):543-50, 2014) (Schrock et al., J Thorac Oncol, 11(9):1493-1502, 2016). Furthermore, genetic amplification of the MET gene has been reported in ˜3% and elevated MET protein expression has been reported in 25% of NSCLC (Bubendorf et al., Lung Cancer, 111:143-9, 2017) (Cappuzzo et al., J Clin Oncol, 27(10):1667-74, 2009) (Fang et al., Oncotarget, 9(16):12959-70, 2018) (Reis et al., Clin Lung Cancer, 19(4): e441-63, 2018) (Sterlacci et al., Virchows Arch, 471(1):49-55, 2017). Increased MET gene amplification and expression is a mechanism of resistance to epidermal growth factor receptor (EGFR)-targeted therapy, and up to 25% of tumors that are resistant to third-generation TKIs may harbor elevated MET by these mechanisms (Bean et al., PNAS, 104(52):20932-7, 2007) (Catenacci et al., Cancer, 123(6):1061-70, 2017) (Go et al., J Thorac Oncol, 5(3):283-9, 2010) (Le et al., JAMA Oncol, 4(2):210-6, 2018) (Zhang et al., Hum Pathol, 72:59-65, 2018).

Tumors harboring MET-amplification or exon-14 deletions respond to MET TKIs (Crizotinib® [package insert], Pfizer Pharmaceutical Company, New York, NY, 2017) (Angevin et al, Eur J Cancer, 87:131-9, 2017) (Camidge et al., J Clin Oncol, 32:abstract 8001, 2014) (Camidge et al., Nat Rev Clin Oncol, 16(6):341-55, 2019) (Paik et al., Cancer Discov, 5(8):842-9, 2015).

REGN5093 is a human bispecific antibody (bsAb) that binds 2 distinct epitopes of MET with nanomolar affinity, blocks HGF binding to MET, and induces internalization and degradation of MET without inducing MET-driven biological responses. In preclinical studies, REGN5093 has shown dose-dependent anti-tumor activity in immune-deficient mouse models of MET-driven cancer including both exon 14-altered and MET-amplified models.

### Unmet Need in Lung Cancer

First-line treatment of advanced NSCLC is guided by the presence of molecular alterations. Patients with tumors which exhibit sensitizing mutations in EGFR, anaplastic lymphoma kinase (ALK), or c-ros oncogene 1 receptor tyrosine kinase (ROS1) fusion are often targeted using small molecule TKIs (Besse et al., Ann Oncol, 25(8): 1475-84, 2014) (Ettinger et al., J Natl Compr Canc Netw 15(4):504-35, 2017) (Reck et al., Ann Oncol, 25 Suppl 3:27-39, 2014). Patients without any of these activating mutations may receive immune-checkpoint-inhibitor antibodies that inhibit the PD-1 receptor or PD-L1, with or without chemotherapy. Beyond these targeted systemic and immunotherapy approaches, advanced NSCLC is treated with platinum-based doublet chemotherapy regimens, with or without maintenance therapy (Besse et al., Ann Oncol, 25(8):1475-84, 2014) (Ettinger et al., J Natl Compr Canc Netw 15(4):504-35, 2017) (Reck et al., Ann Oncol, 25 Suppl 3:27-39, 2014). Long-term survival remains an unmet need in advanced NSCLC. Overall survival is approximately 12 months for PD-1/PD-L1 inhibitors, with some studies in TKI-treated patients exceeding 2 years (Camidge et al., Nat Rev Clin Oncol, 16(6):341-55, 2019).

Anti-PD-1 and anti-PD-L1 therapies have changed the standard of care for many patients with NSCLC (Topalian et al., NEJM, 366(26):2443-54, 2012). However, data is emerging that suggests MET-driven NSCLC patients may not experience equivalent benefit to agents that target the PD-1/PD-L1 axis, even with tumors that express high PD-L1 or exhibit high tumor mutation burden (TMB) (Sabari et al., Ann Oncol, 29(10):2085-91, 2018). This is in keeping with data generated in lung cancers harboring EGFR mutations or ALK rearrangements (Garassino et al., Lancet Oncol, 19(4):521-36, 2018) (Lee et al., JAMA Oncol, 4(2):210-16, 2018) (Peters et al., J Clin Oncol, 35(24):2781-89, 2017) and indicates that monotherapy with anti-PD-1 or anti-PD-L1 may not be the preferred treatment for patients with MET-driven disease. Thus, there remains a substantial unmet need for therapies that improve response rates and survival for patients with MET-altered NSCLC.

### Objectives

The primary objective of the dose escalation (phase 1) part of the study is to assess the safety, tolerability, and pharmacokinetics (PK) of REGN5093 for determination of the maximum tolerated dose (MTD) and/or definition of the recommended phase 2 dose (RP2D) of REGN5093 in patients with MET-altered NSCLC. The secondary objective of the dose escalation (phase 1) part of the study is to assess preliminary anti-tumor activity of REGN5093 as measured by the objective response rate (ORR) per Response Evaluation Criteria in Solid Tumors (RECIST 1.1).

The primary objective of the dose expansion (phase 2) part of the study is to assess preliminary anti-tumor activity of REGN5093 as measured by the ORR per RECIST 1.1. The secondary objectives of the dose expansion (phase 2) part of the study are to assess the safety and tolerability of REGN5093 in each expansion cohort and to assess REGN5093 PK and concentrations in serum.

The secondary objectives of both parts of the study are to assess immunogenicity as measured by anti-drug antibodies (ADA) to REGN5093 and to evaluate other measures of preliminary anti-tumor activity.

The exploratory objectives of both parts of the study are to evaluate relationships between efficacy of REGN5093 and baseline MET alteration/mutation or amplification/expression and/or prior MET TKI treatment across cohorts, to assess pharmacodynamic changes in putative serum or plasma biomarkers, and to evaluate the impact on efficacy of tumor mutational spectrum at baseline and post-treatment in tissue and in circulating tumor DNA (ctDNA expansion phase only).

### Target Population

Adult patients ≥18 years of age (or the legal age of adults to consent to participate in a clinical study per country-specific regulations).

Dose Escalation: Patients with advanced NSCLC exhibiting MET-altered disease by previously documented presence of any of the following: exon 14 gene mutation, MET gene amplification, or elevated MET protein expression. In the dose escalation phase of the study, patients will be enrolled based on documentation of any of the MET alterations defined by any of the above criteria and irrespective of prior experience with MET-targeted TKIs.

Dose Expansion: Patients with advanced NSCLC exhibiting MET-altered disease will be assigned to cohorts based on previously documented presence of the following: MET exon 14-mutated disease and according to prior MET-targeted TKI experience (cohorts 1A MET TKI experienced and 1B no prior MET TKI), high MET gene amplified (cohort 2A no prior MET TKI), high MET protein overexpression (cohort 2B no prior MET TKI), both high MET gene amplified +high MET protein overexpression (cohort 2C no prior MET TKI).

Documented MET status based on at least 1 test will be sufficient to qualify a patient for a relevant cohort; more than 1 category of test is not required. Therefore, cohorts 2A and 2B may include patients with unknown overexpression or gene amplification status, respectively.

The dose expansion phase of the study is designed to further explore the safety and biological activity of REGN5093 at the RP2D. Patients will receive the RP2D of REGN5093 administered IV by a 30-minute infusion.

Patients will be enrolled into separate cohorts according to previously documented MET-altered disease and according to prior MET-targeted TKI experience. The cohorts are designed to provide a relatively homogeneous population of patients according to different cutoffs of the 3 different types of biomarkers: MET exon 14 altered, MET-amplified, and MET protein overexpressed.

Expansion cohorts are as follows (see also Table 10):
Expansion Cohort 1A (MET exon 14 altered NSCLC; MET TKI experienced)
Expansion Cohort 1B (MET exon 14 altered NSCLC; no prior MET TKI)
Expansion Cohort 2A (highly amplified NSCLC MET GCN ≥5 and/or MET/CEP7 ratio ≥2 by FISH or MET GCN >6 by NGS in tissues or MET fold change ≥2 in ctDNA; no prior MET TKI)
Expansion Cohort 2B (high MET protein expression by IHC 3+ or H score of ≥200; no prior MET TKI)
Expansion Cohort 2C (highly amplified NSCLC MET GCN ≥5 and/or MET/CEP7 ratio ≥2 by FISH or MET GCN >6 by NGS in tissues or MET fold change ≥2 in ctDNA and high MET protein overexpressed by IHC 3+ or H score of ≥200; no prior MET TKI) *if <40% tumor content in biopsy is observed then FISH or ctDNA local results must be in the range specified

TABLE 10
Criteria for Assigning Patients to Expansion Cohorts
According to Documented MET-Altered Status

| | MET exon 14 | | MET protein | Prior |
|---|---|---|---|---|
| Cohort | alteration | MET gene amplified | overexpressed | MET TKI |
| Expansion | Yes | Any | Any | experienced |
| Cohort 1A | | | | |
| Expansion | Yes | Any | Any | naïve |
| Cohort 1B | | | | |
| Expansion | No* | High: MET GCN ≥5 | Any | naïve |
| Cohort 2A | | and/or MET/CEP7 | | |
| | | ratio ≥2 by FISH or | | |
| | | MET GCN ≥6 by | | |
| | | NGS in tissues or | | |
| | | MET fold change ≥2 | | |
| | | in ctDNA | | |
| Expansion | No* | Any | High: IHC 3+ or | naïve |
| Cohort 2B | | | H score ≥200 | |
| Expansion | No* | High: MET GCN ≥5 | High: IHC 3+ or | naïve |
| Cohort 2C | | and/or MET/CEP7 | H score ≥200 | |
| | | ratio ≥2 by FISH or | | |
| | | MET GCN ≥6 by | | |
| | | NGS in tissues or | | |
| | | MET fold change ≥2 | | |
| | | in ctDNA | | |

CEP7: centromeric portion of chromosome 7
ctDNA: circulating tumor DNA
GCN: gene copy number
IHC: immunohistochemistry
MET: mesenchymal epithelial transition
TKI: tyrosine kinase inhibitors
*unless cohort 1B is already filled
** if <40% tumor content in biopsy is observed then FISH or ctDNA local results must be in the range specified

Patients will not be required to have previously undertaken testing for all 3 categories of MET-altered disease in order to qualify for a relevant expansion cohort. For dose expansion, in cases where patients are MET-TKI naïve and have data from more than one type of test for MET-altered disease indicating that they could qualify for more than one cohort, they will be assigned to expansion cohorts (if slots available) with the following prioritization:
First priority cohorts: MET exon 14 alterations =cohorts 1A and 1B
Second priority (MET TKI-naïve patients without MET exon 14 alterations, or patients with exon 14 alterations only after cohort 1B is filled): MET-gene amplified +protein expression will be considered for assignment to cohorts 2A, 2B, and 2C.

Expansion cohorts will have a Simon 2-stage design. The enrollment of expansion cohorts will be paused after the number of patients required for stage 1 is enrolled into the cohort. The cohort will either be stopped or expanded pending the number of responders observed in stage 1 and the corresponding stage 1 criteria.

### Inclusion Criteria

A patient must meet the following criteria to be eligible for inclusion in the study:
Histologically confirmed NSCLC that is at advanced stage and for which there is no standard therapy option likely to convey clinical benefit. Advanced is defined as unresectable or metastatic disease. Patients must have exhausted all approved available therapies appropriate for the patient.
Has available archival tumor tissue, unless discussed with the medical monitor.
Previously documented presence of:
   For dose escalation cohorts: either MET-exon14 gene mutation and/or MET gene amplification (by any local CLIA laboratory MET amplification call), and/or elevated MET protein expression (IHC ≥2+ or H score of >150)
   For dose expansion cohorts 1 A and 1B: MET exon 14-mutation; MET TKI experienced or no prior MET TKI, respectively
   For dose expansion cohort 2A: MET gene highly amplified (MET GCN ≥5 and/or MET/CEP7 ratio ≥2 by FISH or MET GCN ≥6 by NGS in tissues or MET fold change ≥2 in ctDNA); no prior MET TKI
   For dose expansion cohort 2B: MET protein highly overexpressed (IHC 3+ or H score of ≥200); no prior MET TKI)
   For dose expansion cohort 2C: MET gene highly amplified (MET GCN ≥5 and/or MET/CEP7 ratio >2 by FISH or MET GCN ≥6 by NGS in tissues or MET fold change ≥2 in ctDNA) and MET protein highly overexpressed (IHC 3+or H score of ≥200); no prior MET TKI *if <40%tumor content in biopsy is observed then FISH or ctDNA local results must be in the range specified
Willing to provide tumor tissue from newly obtained biopsy. Newly obtained biopsies at screening are required unless medically contra indicated and discussed with the medical monitor. For patients in expansion cohorts, biopsies should be taken from tumor site which has not been irradiated previously and is not the only measurable target lesion.
For expansion cohorts only: At least 1 lesion that is measurable by RECIST 1.1. Tumor lesions in a previously irradiated area are considered measurable if progression has been demonstrated in such lesions after radiation.
Eastern Cooperative Oncology Group (ECOG) performance status of 0 or 1
Adequate organ and bone marrow function documented by:
Hemoglobin ≥9.0 g/dL
Absolute neutrophil count ≥1.5×10⁹/L
Platelet count ≥75×10⁹/L
Either serum creatinine ≤1.5 × ULN or estimated glomerula filtration rate (GFR) ≥30 mL/min/1.73m²
Adequate hepatic function:
Total bilirubin ≤1.5 x ULN (≤3 × ULN if tumor liver involvement)
AST ≤2.5 × ULN (≤5 × ULN if tumor liver involvement)
ALT ≤2.5 × ULN (≤5 × ULN if tumor liver involvement)
Alkaline Phosphatase ≤2.5 × ULN (≤5 × ULN if tumor liver or bone involvement)

### NOTES:

(a) Patients with tumor liver involvement if levels of AST ≥3 × ULN or ALT ≥3 × ULN, and bilirubin levels ≥2 × ULN, will be excluded regardless of the above criteria.
(b) Patients with Gilbert's syndrome do not need to meet total bilirubin requirements provided their total bilirubin is not greater than their historical level. Gilbert's syndrome must be documented appropriately as past medical history.
Adult patients ≥18 years of age (or the legal age of adults to consent to participate in a clinical study per country-specific regulations).
Willing and able to comply with clinic visits and study-related procedures and requirements
Must be willing and able to provide informed consent as specified by health authorities and institutional guidelines
Provide informed consent signed by study patient or legally acceptable representative

### Exclusion Criteria

A patient who meets any of the following criteria will be excluded from the study:
Has received treatment with an approved systemic therapy or has participated in any study of an investigational agent or investigational device according to the following timeframe:
   For small molecule cytotoxins or other agents unlikely to interact with study drug: within 2 weeks or 5 half-lives of the prior treatment whichever is shorter with a minimum of 7 days from the first dose of study therapy.
   Exception: Patients who have received or are enrolled in a study involving treatment with an investigational immunoPET reagent are not excluded.
Has not yet recovered (i.e., grade ≤1 or baseline) from any acute toxicity from prior therapy except for:
   laboratory changes as described in inclusion criteria, and
   patients with grade ≤2 neuropathy
For immune-related AEs affecting any organ system within 2 months prior to enrollment there must be documentation of a trajectory of improvement of this irAE (to grade ≤1 or baseline by the time of enrollment) AND for those toxicities that remain at grade 1, documentation of 2 stable evaluations separated by at least 4 weeks.
NOTE: Endocrine immune-mediated AEs controlled with hormonal or other non-immunosuppressive therapies without resolution prior to enrollment are allowed
Has received radiation therapy or major surgery within 14 days of first administration of study drug or has not recovered (ie, grade ≤1 or baseline) from AEs, except for laboratory changes as described in inclusion criteria and patients with grade ≤2 neuropathy
For expansion cohorts only: prior treatment with MET-targeted biologic therapy (function-blocking antibodies or ADCs). In addition, for expansion cohorts 1B, 2A, 2B, and 2C, prior treatment with any MET-targeted agent including small molecule tyrosine kinase inhibitors e.g., crizotinib, capmatinib, tepotinib
For expansion cohorts only: Another malignancy, with the following exceptions:
   Non-melanoma skin cancer that has undergone potentially curative therapy or
   In situ cervical carcinoma or
   Any other tumor that has been treated, and the patient is deemed is be in complete remission for at least 2 years prior to enrollment and no additional therapy is required during the study period
Untreated or active primary brain tumor, CNS metastases, leptomeningeal disease or spinal cord compression
   Exception: Patients with previously treated central nervous system metastases or spinal cord compression may participate provided:
   No evidence of progression for at least 2 weeks prior to the first dose of study therapy, and any neurologic symptoms have returned to baseline
Encephalitis, meningitis, organic brain disease (e.g., Parkinson's disease), or uncontrolled seizures in the year prior to first dose of study therapy
Uncontrolled infection with human immunodeficiency virus, hepatitis B or hepatitis C infection; or diagnosis of immunodeficiency

### NOTES:

(a) Patients with known HIV infection who have controlled infection (undetectable viral load [HIV RNA PCR] and CD4 count above 350 either spontaneously or on a stable antiviral regimen) are permitted. For patients with controlled HIV infection, monitoring will be performed per local standards.
(b) Patients with known hepatitis B (HepBsAg+) who have controlled infection (serum hepatitis B virus DNA PCR that is below the limit of detection AND receiving anti-viral therapy for hepatitis B) are permitted. Patients with controlled infections must undergo periodic monitoring of HBV DNA. Patients must remain on anti-viral therapy for at least 6 months beyond the last dose of investigational study drug.
(c) Patients who are known hepatitis C virus antibody-positive (HCV Ab+) who have controlled infection (undetectable HCV RNA by PCR either spontaneously or in response to a successful prior course of anti-HCV therapy) are permitted.
Any infection requiring hospitalization or treatment with IV anti-infectives within 2 weeks prior to first dose of study therapy.
Placeholder for deleted exclusion criteria
Placeholder for deleted exclusion criteria
Known psychiatric or substance abuse disorders that would interfere with participation with the requirements of the study
Any medical condition, co-morbidity, physical examination finding, or metabolic dysfunction, or clinical laboratory abnormality that, in the opinion of the investigator, renders the patient unsuitable for participation in a clinical trial due to high safety risks and/or potential to affect interpretation of results of the study
Women with a positive serum hCG pregnancy test at the screening/baseline visit. Breastfeeding women are also excluded
Women of childbearing potential* or men who are unwilling to practice highly effective contraception prior to the initial dose/start of the first treatment, during the study, and for at least 6 months after the last dose. Highly effective contraceptive measures include:
   Stable use of combined (estrogen- and progestogen-containing) hormonal contraception (oral, intravaginal, transdermal) or progestogen-only hormonal contraception (oral, injectable, implantable) associated with inhibition of ovulation initiated 2 or more menstrual cycles prior to screening
   Intrauterine device (IUD); intrauterine hormone-releasing system (IUS)
   Bilateral tubal ligation
   Vasectomized partner (provided that the male vasectomized partner is the sole sexual partner of the study participant and that the partner has obtained medical assessment of surgical success for the procedure)
   And/or sexual abstinencet\, ‡
*Women of childbearing potential are defined as women who are fertile following menarche until becoming post-menopausal, unless permanently sterile. Permanent sterilization methods include hysterectomy, bilateral salpingectomy, and bilateral oophorectomy.
A post-menopausal state is defined as no menses for 12 months without an alternative medical cause. A high follicle stimulating hormone (FSH) level in the post-menopausal range may be used to confirm a post-menopausal state in women not using hormonal contraception or hormonal replacement therapy. However, in the absence of 12 months of amenorrhea, a single FSH measurement is insufficient to determine the occurrence of a post-menopausal state. The above definitions are according to Clinical Trial Facilitation Group (CTFG) guidance.
Pregnancy testing and contraception are not required for women with documented hysterectomy or tubal ligation.
† Sexual abstinence is considered a highly effective method only if defined as refraining from heterosexual intercourse during the entire period of risk associated with the study drugs. The reliability of sexual abstinence needs to be evaluated in relation to the duration of the clinical trial and the preferred and usual lifestyle of the patient.
‡ Periodic abstinence (calendar, symptothermal, post-ovulation methods), withdrawal (coitus interruptus), spermicides only, and lactational amenorrhea method (LAM) are not acceptable methods of contraception. Female condom and male condom should not be used together.

### Methods—Study Design

Patients with documentation of at least 1 of 3 main categories of MET alterations will be included in this First in Human (FIH) open-label study: exon 14 skipping mutation, MET gene amplification (MET GCN >5 and/or MET/CEP7 ratio >2 by FISH or MET GCN ≥6 by NGS in tissues or MET fold change ≥2 in ctDNA), and MET protein overexpression (immunohistochemistry 3+ or H score ≥200). Patients will not be required to have previously undertaken all 3 categories of tests for MET-altered disease.

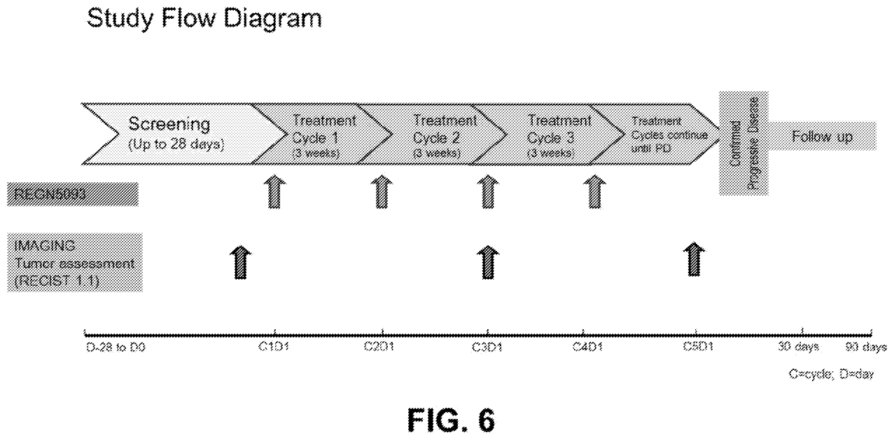

FIG. 6 depicts the study flow from the initial screening period to the post-treatment follow-up.

### Description of Study Cohorts and Dose Escalation

Patients underwent screening procedures to determine eligibility within 28 days prior to the initial administration of REGN5093 and subsequent to signing the informed consent form (ICF).

### Dose Escalation

In the dose escalation phase, a series of 3 DLs of REGN5093 was investigated: 500, 1000, and 2000 mg administered IV once every three weeks (Q3W) by a 30-minute infusion (see Table 11).

TABLE 11
Dose Escalation

| | Dose (mg) | |
|---|---|---|
| Dose Level | Q3W | Initial enrollment numbers |
| DL-1¹ | 250 | 3 to 4 |
| DL1 | 500 | 3 to 4 |
| DL1a² | 750 | 3 to 4 |
| DL2 | 1000 | 3 to 4 |
| DL2a² | 1500 | 3 to 4 |
| DL3 | 2000 | 3 to 4 |

¹DL-1 may be used if DL1 proves intolerable
²DL1a and DL2a are optional dose levels to be explored in the event that the dose immediately higher is not tolerable and the dose immediately lower shows insufficient clinical and PK signal.

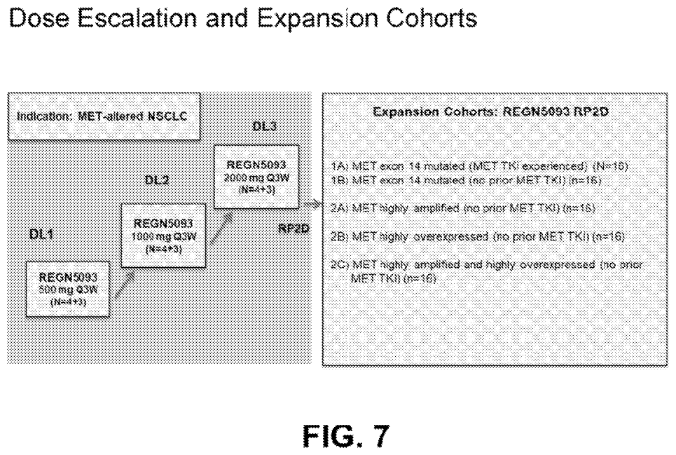

A modified 3+3 dose escalation design (“4+3”) will be utilized (Le Tourneau et. al., J Natl Cancer Inst, 101(10):708-20, 2009). The dose escalation will proceed until an MTD is attained or a dose is selected for expansion based on safety/tolerability and sufficient evidence of response (RP2D). Dose de-escalation (DL-1) cohort and intermediate cohorts may be enrolled to explore intermediate doses should the initial or subsequent level, respectively be deemed not tolerated. To further evaluate safety and collect biologic information, up to 6 additional patients may, at the discretion of the sponsor (in consultation with investigators), be enrolled at any DL deemed to be tolerated. A diagram of the study design is shown in FIG. 7.

The dose limiting toxicity (DLT) evaluation period will be 21 days starting with cycle 1 day 1. Although a minimum of 3 patients at each DL will be required to be evaluable for DLT, to maximize the efficiency of the phase 1 dose escalation while maintaining patient safety, 4 patients will be enrolled at each DL in case a patient discontinues prior to being evaluable for DLT. The rules for tolerability are as follows:

Tolerability of a DL will be considered achieved if all potentially DLT-evaluable patients complete the 21-day DLT period without a DLT (0 in 3 patients or 0 in 4 patients).

Note: If 3 patients complete the DLT period without experiencing a DLT, but there is a fourth patient in the DLT evaluation period, tolerability of the DL will only be considered achieved when the fourth patient completes the DLT evaluation period or discontinues therapy prior to being evaluable for DLT.

If there is 1 DLT in either 3 or 4 DLT-evaluable patients, then 4 or 3 more patients, respectively, will be enrolled, for a total of 7 patients. A dose will be considered tolerable if there is 1 DLT in 6 or 7 patients. The MTD is reached if there are 2 or more DLTs in 2 to 7 evaluable patients.

At the highest DL tolerated, to evaluate further safety, an additional 3 to 4 patients may be enrolled for a total of 6 to 10 DLT-evaluable patients. The dose will be considered acceptable if there is 0 to 1 DLT in 6 to 8 patients, or up to 2 DLTs in 9 to 10 patients.

Escalation to the next dose cohort will occur once all of the initial 3 or 4 patients enrolled in a cohort have been observed for at least 21 days and completed safety assessments on day 22 (cycle 2 day 1), and the data have been reviewed at a Dose Escalation Review meeting.

After the required number of patients is enrolled in a given dose cohort, the enrollment will be paused for DLT evaluation (although screening for the next dose cohort may begin prior to confirmation that the current dose is safe). The Dose Escalation Review meeting will be led by a designated member of the sponsor's clinical team (generally either the medical or study director) and at a minimum will be attended by the sponsor's medical/study director and the Global Patient Safety Lead; other individuals, including the investigators, may be included. The dose cohort will be stopped, expanded, or escalated per dose escalation criteria.

### Dose-Limiting Toxicities

Dose-limiting toxicity (DLT) is any toxicity that could preclude advancing to the higher doses that have been specified in this protocol. The DLT observation period for determination of safety for dose escalation is defined as 21 days starting with cycle 1, day 1, with the intent to monitor the safety and tolerability of the first dose of REGN5093. To be evaluable for a DLT, a patient must have:
Received at least 1 dose of study drug and be monitored for at least 21 days following the first administration of study drug,
Or experienced a DLT (defined below) prior to the completion of the DLT period.

The duration of the DLT observation period may be longer for patients experiencing an AE for which the duration must be assessed in order to determine if the event was a DLT.

Regardless of whether a patient remains on study treatment and/or continues to participate in study procedures, such an event will count as a DLT for the involved cohort if the event occurs during the DLT observation period.

### Dose-Limiting Toxicity Definition

A DLT in general will be defined as any of the following treatment-emergent toxicities, excluding toxicities clearly related to disease progression or inter-current illness. The grades for these toxicities are defined according to CTCAE version 5.0.

### Hematologic Toxicity:

Grade 4 neutropenia lasting >7 days
Grade 4 thrombocytopenia
Grade 4 anemia
Grade 3 thrombocytopenia with bleeding
Grade ≥3 febrile neutropenia (fever ≥38.5° C. with absolute neutrophil count [ANC]<1.0×10⁹/L) or grade ≥3 neutropenia with documented infection

### Non-Hematologic Toxicity:

Non-hematologic grade ≥3 toxicity except for:
   a. Alopecia
   b. Grade 3 nausea, vomiting, or diarrhea unless persistent (>72 hours duration), after initiation of supportive care measures as prescribed by the treating physician
   c. Clinically insignificant laboratory abnormalities
Clinically significant grade ≥3 laboratory values which require medical intervention or leads to hospitalization
Liver dysfunction consistent with Hy's law (Temple, J Allergy Clin Immunol, 117(2):391-97, 2006) or ALT or AST >3× ULN and bilirubin >2× ULN

The frequency, time to onset, and severity of toxicities, as well as the success of standard medical management and dosing interruptions/delays will be analyzed to determine if a given toxicity should be considered a DLT for dose escalation purposes.

In general, as there is limited clinical experience for the new biologic molecule REGN5093, any AE will be treated as unexpected.

Treatment-emergent adverse events that appear to meet the DLT definition will be discussed between the sponsor and the investigator. The final decision of whether or not the TEAE meets the DLT definition will be based on a careful review of all relevant data and consensus between the medical/study director and the designated safety lead from the Global Patient Safety department. The investigator may also be consulted.

Regardless of whether a patient remains on study treatment and/or continues to participate in the study procedures, an event that meets the DLT criteria will count as a DLT for the involved cohort if the event occurs during the DLT observation period.

### Maximum Tolerated Dose

The MTD is defined as the DL immediately below the level at which dosing is stopped due to the occurrence of 2 or more DLTs out of up to 7 evaluable patients. If the study is not stopped due to the occurrence of a DLT, it will be considered that the MTD has not been determined.

If the MTD is not reached, the RP2D for further evaluation may be chosen based upon clinical, PK, and/or biomarker data that indicate a pharmacologically active dose has been reached in conjunction with available safety information.

REGN5093 will be supplied as a lyophile in sterile, single-use vials. During the trial a new presentation is planned to be introduced, a sterile solution. Each vial will contain REGN5093 at a concentration of 25 mg/ml. Both presentations will include a labeled vial in a labeled carton.

Instructions on dose preparation are provided in the pharmacy manual.

No pre-medication is needed prior to administration of REGN5093. REGN5093 will be administered by IV infusion over 30 minutes Q3W.

### Prohibited Medications

While participating in this study, a patient may not receive any standard or investigational agent for treatment of a tumor other than REGN5093, per the study's specified dosing regimens.

Systemic treatments for cancer are not allowed during the study period.

Patients must not receive live vaccines during the study.

Radiotherapy is not allowed during the study period with the exception of the following: after communication with the sponsor, focal palliative treatment (e.g., radiation) would be allowed for local control of a tumor. Palliative radiation therapy for pain management at sites of bone disease or lesion sites in the brain are allowed (as long as the lesions are not followed for treatment response evaluation) after discussion with the sponsor.

Any other medication which is considered necessary for the patient's welfare, and which is not expected to interfere with the evaluation of the study drug, may be given at the discretion of the investigator.

### Permitted Medications

Gonadotropin-releasing hormone agonist therapy may be continued and is not prohibited. Hormone-replacement therapy is allowed. Inhaled, topical, ophthalmologic, or intranasal steroids are allowed. Treatments for bone metastases (bisphosphonates, denosumab) and systemic corticosteroids are allowed. Use of high doses of steroids over a prolonged period of time require discussion with the medical monitor.

### Safety

The safety and tolerability of REGN5093 will be monitored by clinical assessment of AEs, physical examinations (complete and limited), repeated measurements of vital signs (temperature, blood pressure, pulse, and respiration), 12-lead electrocardiograms (ECGs), and laboratory assessment including standard hematology, chemistry, and urinalysis. Vital signs, including temperature, sitting blood pressure, pulse, and respiration will be collected pre-dose at timepoints.

An AE is any untoward medical occurrence in a patient administered a study drug which may or may not have a causal relationship with the study drug. Therefore, an AE is any unfavorable and unintended sign (including abnormal laboratory finding), symptom, or disease which is temporally associated with the use of a study drug, whether or not considered related to the study drug (ICH E2A Guideline. Clinical Safety Data Management: Definitions and Standards for Expedited Reporting, October 1994).

An SAE is any untoward medical occurrence that at any dose:
Results in death—includes all deaths, even those that appear to be completely unrelated to study drug (e.g., a car accident in which a patient is a passenger).
Is life-threatening—in the view of the investigator, the patient is at immediate risk of death at the time of the event. This does not include an AE that had it occurred in a more severe form, might have caused death.
Requires in-patient hospitalization or prolongation of existing hospitalization. In-patient hospitalization is defined as admission to a hospital or an emergency room for longer than 24 hours. Prolongation of existing hospitalization is defined as a hospital stay that is longer than was originally anticipated for the event, or is prolonged due to the development of a new AE as determined by the investigator or treating physician.
Results in persistent or significant disability/incapacity (substantial disruption of one's ability to conduct normal life functions)
Is a congenital anomaly/birth defect
Is an important medical event. Important medical events may not be immediately life-threatening or result in death or hospitalization, but may jeopardize the patient or may require intervention to prevent one of the other serious outcomes listed above (e.g., intensive treatment in an emergency room or at home for allergic bronchospasm; blood dyscrasias or convulsions that do not result in hospitalization; or development of drug dependency or drug abuse).

Hospitalization or death due solely to manifestations consistent with typical progression of underlying malignancy will not be considered an SAE.

### Efficacy

Radiographic tumor response will be used to determine overall response for each patient defined by RECIST 1.1 (Eisenhauer et al., Eur J Caner, 45(2):228-47, 2009). Radiographic disease assessment will inform the calculation of:
ORR, defined as the percentage of patients with a BOR of complete response (CR) or partial response (PR)
DOR, defined as time from first response of CR or PR to first radiographic progression or death due to any cause for patients with confirmed CR or PR. In the absence of radiographic progression or death before the analysis cutoff date or the date of initiation of a further anti-cancer treatment, the DOR will be censored at the date of the last valid response assessment not showing progression performed prior to the analysis cutoff date or initiation of a further anti-cancer treatment, whichever is earlier.
DCR, defined as the percentage of patients with a BOR of CR, PR, or stable disease (SD)
PFS defined as time from first study treatment administration to first radiographic progression or death due to any cause. The same censoring rule as DOR will be used.
OS defined as the time from first study treatment administration to death due to any cause. For patients who have not died, OS will be censored at the last date that patient is known to be alive. OS will be assessed based on survival data reported by the investigators.

Diagnostic quality CT with contrast and contrast-enhanced MRI are the preferred imaging modalities for assessing radiographic tumor response. In patients in whom contrast is strictly contraindicated, non-contrasted CT scans of the chest and non-contrast MRI scans of the body except chest will suffice. The chest, abdomen, and pelvis must be imaged along with any other known or suspected sites of disease. If more than 1 imaging modality is used at screening, the most accurate imaging modality according to RECIST 1.1 (Appendix 1) should be used when recording data. The same imaging modality and techniques used at screening should be used for all subsequent assessments.

At screening, MRI or CT of the brain with contrast (unless contraindicated then MRI without contrast) should be performed in patients with a known history of treated brain metastasis.

Additional sites of known disease should be imaged at screening.

A diagnostic quality (≤5 mm slices) CT scan with contrast of the chest and abdomen as well as any other known sites of disease (e.g., neck), will be performed at screening, on day 1 of treatment cycles 2 and 3, every three months post last visit, and at any time when disease progression is suspected. Scans will include description of tumor locations, and up to 5 of the largest dominant disease masses (no more than 2 per organ) should be chosen as target lesions and measured by longest diameter if non-nodal and short axis for nodal lesions, per RECIST 1.1. All lesions should be assessed and documented. If a CT scan is not feasible, an MRI scan may be performed.

For each patient, the same method of measurements and the same technique must be used to evaluate each lesion throughout the study. If a patient inadvertently misses a prescribed tumor evaluation or a technical error prevents the evaluation, the patient may continue treatment until the next scheduled assessment, unless signs of clinical progression are present. If, at any time during the treatment phase, there is suspicion of disease progression based on clinical or laboratory findings (and before the next scheduled assessment), an unscheduled tumor assessment should be performed.

### Procedures and Assessments

Anti-tumor activity will be assessed by computed tomography (CT) or magnetic resonance imaging (MRI). The safety and tolerability of REGN5093 will be monitored by clinical assessment of AEs, physical examinations (complete and limited), repeated measurements of vital signs (temperature, blood pressure, pulse, and respiration), 12-lead electrocardiograms (ECGs), and laboratory assessment including standard hematology, chemistry, and urinalysis.

Blood will be collected to assess REGN5093 PK and concentrations in serum and immunogenicity (ADA) in serum; and for additional biomarker assessments. Additional biomarkers will be measured in serum or plasma. Exploratory predictive and pharmacodynamic biomarkers related to REGN5093 treatment exposure, clinical activity, or underlying disease will be investigated using samples from collected serum, plasma, archived tumor tissue, and on-study tumor biopsy tissue, tumor DNA (including circulating tumor DNA), and tumor RNA samples.

### Study Endpoints

The primary endpoints of the dose escalation (phase 1) part of the study are:
Safety, as measured by the incidence and severity of treatment-emergent adverse events (TEAEs)/adverse events of special interest (AESIs)/serious adverse events (SAEs), and grade ≥3 laboratory abnormalities during the treatment period and up to 90 days after the last dose
Tolerability, as measured by the incidence of dose-limiting toxicities (DLTs) from the first dose through the end of the DLT observation period for REGN5093
REGN5093 concentrations in serum over time

The primary endpoint of the dose expansion (phase 2) part of the study is ORR per RECIST 1.1, defined as the percentage of patients with a best overall response (BOR) of confirmed CR or PR according to RECIST 1.1 criteria.

The secondary endpoints of the dose escalation part of the study are: ORR per RECIST 1.1.

The secondary endpoints of the dose expansion part of the study are:
Safety, as measured by the incidence and severity of TEAEs, AESIs/SAEs, and grade ≥3 laboratory abnormalities
REGN5093 PK and concentrations in serum over time

The secondary endpoints for both phases of the study are:
Duration of response (DOR) per RECIST 1.1.
Disease control rate (DCR) per RECIST 1.1.
Progression free survival (PFS) per RECIST 1.1.
OS
Immunogenicity as measured by ADA to REGN5093.

The exploratory endpoints for both parts of the study are:
The response to REGN5093 by type (and extent) of baseline MET alteration and prior MET-targeted TKI experience
The response to REGN5093 by baseline tumor mutational status

### Example 8. Safety, Tolerability, and Efficacy of REGN5093 in Patients with MET Altered Advanced NSCLC

REGN5093 has a therapeutic benefit in patients with MET-altered NSCLC and showed promising efficacy signals with a tolerable safety profile.

REGN5093 was investigated as monotherapy and was administered intravenously once every 3 weeks in dose escalation cohorts (phase 1) followed by an expansion phase (phase 2). For each patient, the study consisted of a screening period of up to 28 days, followed by 3-week cycles of REGN5093 monotherapy. REGN5093 at 2000 mg was the recommended Phase 2 dose; prior dose levels were 500 mg and 1000 mg.

Tumor measurements were performed at baseline and every 6 weeks until disease progression, withdrawal of consent, death, or initiation of another anti-cancer treatment.

Tumor tissue (archival and on-study tumor biopsies) were obtained and utilized for retrospective analysis of MET alterations (and additional biomarker analyses, tissue permitting). Newly obtained biopsies at screening were also required unless considered unsafe.

Sixty-nine patients received REGN5093 in both the dose-escalation and dose-expansion phases. Patient characteristics were consistent with a heavily treated population, with a median of 2.5 prior lines of therapy (range: 1-8). Most patients had an ECOG PS of 1 (80%). Most patients had non-squamous histology (93.6%), and EGFR mutation was present in 37.2% of patients (Table 12). The study population had a median age of 66 years, 53.8% were male, 70.5% were Asian.

TABLE 12
Baseline Demographics and Clinical Characteristics

| Characteristic | | Total (N = 78) | |
|---|---|---|---|
| Age | Median (range), years | 66 | (30-85) |
| | ≥65 year, n(%) | 46 | (59.0) |
| Sex, n(%) | Male | 42 | (53.8) |
| | Female | 36 | (46.2) |
| Race, n(%) | Asian | 55 | (70.5) |
| | White | 11 | (14.1) |
| | Black | 1 | (1.3) |
| | Not reported/missing | 11 | (14.1) |
| ECOG PS, n (%) | 0 | 15 | (19.2) |
| | 1 | 63 | (80.8) |
| Histology, n (%) | Non-squamous | 73 | (93.6) |
| | Squamous | 5 | (6.4) |
| Metastases, n (%) | Liver | 22 | (28.2) |
| | Brain | 22 | (28.2) |
| EFGR status, n (%) | Wild type | 36 | (46.2) |
| | Mutant | 29 | (37.2) |
| Number of prior | Unknown | 13 | (16.7) |
| lines of therapy | Median, (range) | 2.5 | (1-8) |

Safety data

No dose-limiting toxicities (DLTs) were observed. REGN5093 demonstrated a similar safety profile in the dose-escalation and dose-expansion phases, notably with grade 1/2 peripheral oedema in only six (9%) patients (Table 13). At the time of the data cut-off, nine (13%) patients were still on treatment and 60 (87%) patients had discontinued treatment. The primary reasons for discontinuation of treatment were: Disease progression in 52 (75%) patients; Patient decision in five (7%) patients; Adverse event in only three (4%) patients.

TABLE 13
Safety Summary
Total (N = 69)

| | Duration of exposure, median (range), weeks | | | |
|---|---|---|---|---|
| | 9.3 (1-82) | | | |
| | Any grade | | Grade 3-5 | |
| TEAEs, n (%) | | | | |
| Overall | 59 | (86) | 18 | (26) |
| Serious | 16 | (23) | 12 | (17) |
| Led to discontinuation | 3 | (4) | 2 | (3) |
| Led to death | 1 | (1) | 1 | (1) |

TEAEs in ≥5% of patients

| Nausea | 9 | (13) | 0 | |
|---|---|---|---|---|
| Fatigue | 7 | (10) | 1 | (1) |
| Oedema peripheral | 6 | (9) | 0 | |
| Pruritis | 6 | (9) | 0 | |
| ALT increased | 5 | (7) | 2 | (3) |
| AST increased | 5 | (7) | 1 | (1) |
| COVID-19 | 5 | (7) | 1 | (1) |
| Constipation | 5 | (7) | 0 | |
| Dizziness | 5 | (7) | 0 | |
| Headache | 5 | (7) | 0 | |
| Insomnia | 5 | (7) | 0 | |
| Pneumonia | 4 | (6) | 3 | (4) |
| Back pain | 4 | (6) | 3 | (4) |
| Decreased appetite | 4 | (6) | 0 | |
| Dyspnea | 4 | (6) | 2 | (3) |
| Hypoalbuminaemia | 4 | (6) | 0 | |
| Musculoskeletal chest pain | 4 | (6) | 0 | |
| Pleural effusion | 4 | (6) | 2 | (3) |
| Pulmonary embolism | 4 | (6) | 1 | (1) |
| Vomiting | 4 | (6) | 0 | |
| Treatment-related AEs | | | | |
| Overall | 27 | (39) | 3 | (4) |
| Serious | 2 | (3) | 2 | (3) |

AE, adverse event; ALT, alanine aminotransferase; AST, aspartate aminotransferase; TEAE, treatment-emergent adverse event.

### Tumor Response

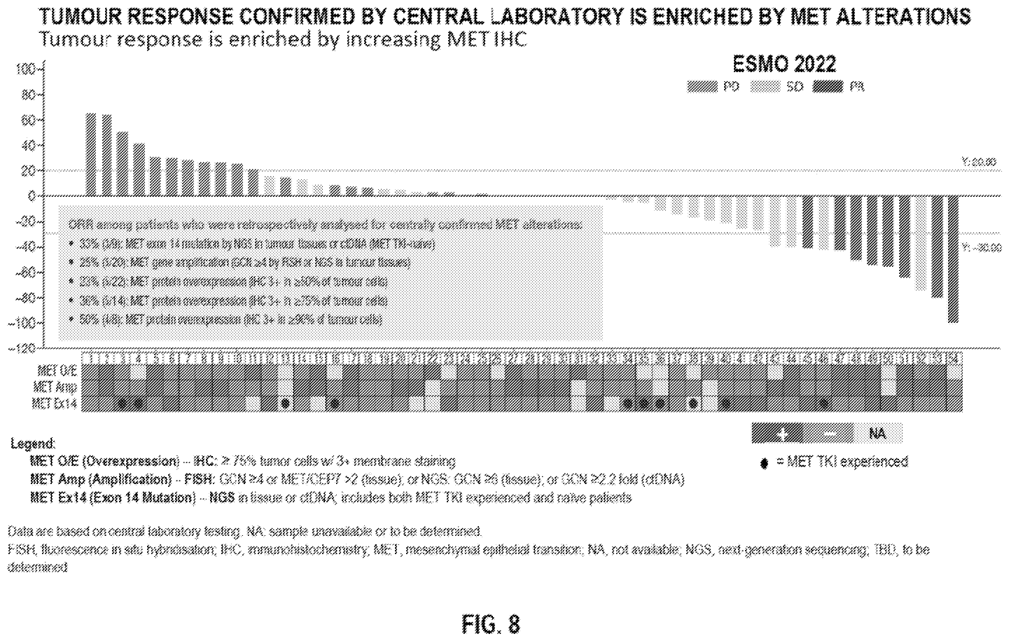

Partial responses (by investigator assessment) were observed in a subset of patients with exon 14 alteration in DNA or a deletion that leads to exon 14 skipping who were naive to MET TKI treatment, and in patients with MET gene amplification and/or MET protein overexpression (Table 14 and FIG. 8).

TABLE 14
Best overall response rate (ORR) among patients who received the 2000-mg
dose Q3W of REGN5093^{†} (MET alteration based on local records)

| | | | Confirmed | | |
|---|---|---|---|---|---|
| | | | responses | | |
| | | Number | (all partial | | |
| | Total number | evaluable | responses) | ORR | |
| Exon 14 alteration in | 11 | 11 | 0 | 0% | (0/11) |
| DNA or a deletion that | | | | | |
| leads to exon 14 | | | | | |
| skipping (MET TKI | | | | | |
| experienced) | | | | | |
| Exon 14 alteration in | 10 | 10 | 3 | 30% | (3/10) |
| DNA or a deletion that | | | | | |
| leads to exon 14 | | | | | |
| skipping (MET TKI | | | | | |
| naïve) | | | | | |
| Patients with PD-(L)1 | | 7 | 2 | | |
| experience | | | | | |
| Patients with prior | | 1 | 1 | | |
| EGFR inhibitor and no | | | | | |
| PD-(L1) experience | | | | | |
| MET gene | 40 | 35 | 5 | 12.5% | (5/40) |

| | | |
|---|---|---|
| amplification and/or | | |
| MET protein | | |
| overexpression (MET | | |
| TKI naïve) | | |
| Patients with PD-(L)1 | 19 | 3 |
| experience | | |
| Patients with prior | 13 | 2 |
| EGFR inhibitor and no | | |
| PD-(L)1 experience | | |

^{†}Including all expansion cohort patients and three patients from dose escalation cohort 3 at the 2000-mg dose level.
EGFR, epidermal growth factor receptor; MET, mesenchymal epithelial transition; PD-(L)1, programmed cell death-(ligand) 1; PR, partial response; TKI, tyrosine kinase inhibitor.

ORR among patients with centrally confirmed MET alterations: ·33% (3/9): MET exon 14 mutation by NGS in tumor tissues or ctDNA (MET TKI-naïve) ·25% (5/20): MET gene amplification (GCN ≥5 by FISH or NGS in tumor tissues) ·23% (5/22): MET protein overexpression (IHC 3+ in ≥50% of tumor cells) ·36% (5/14): MET protein overexpression (IHC 3+ in ≥75% of tumor cells) ·50% (4/8): MET protein overexpression (IHC 3+ in ≥90% of tumor cells).

### Pharmacokinetics

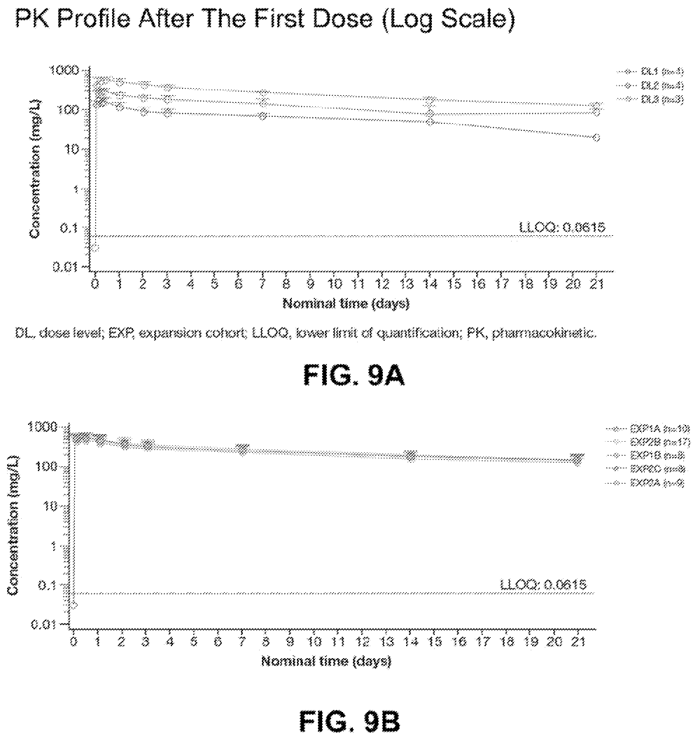

REGN5093 exposure in serum appeared linear and dose-proportional over a dose range of 500 mg to 2000 mg Q3W intravenous (IV), and concentrations in serum at 2000 mg Q3W IV was similar in the dose-escalation cohort and across the various expansion cohorts (see FIG. 9). The elimination half-life estimated by non-compartmental analysis over a 3-week dosing interval was 15 days.

### Conclusions

Among this population of heavily treated patients with MET-altered advanced NSCLC, REGN5093 monotherapy demonstrated an acceptable safety profile. No DLTs were observed. Eighty-six percent of patients experienced TEAEs of any grade. Twenty-six percent of patients experienced Grade ≥3 TEAEs. Three (4%) patients discontinued treatment due to a TEAE. REGN5093 exposure in serum appeared to increase in a dose-dependent manner. REGN5093 monotherapy demonstrated preliminary efficacy signals among patients with MET exon 14 alteration in DNA or a deletion that leads to exon 14 skippings as well as patients with MET gene amplification and/or MET protein overexpression. Tumor response was enhanced with centrally confirmed biomarker selection.

### Example 9: Predictive Biomarkers of Response to REGN5093

In some instances, when treating MET-altered advanced non-small cell lung cancer (aNSCLC), patient selection can be guided by the use of predictive biomarkers of response to REGN5093. REGN5093 monotherapy was investigated in patients with MET-altered aNSCLC. Tumor measurements were performed at baseline and Q6W until progression, consent withdrawal, death, or initiation of another anti-cancer treatment.

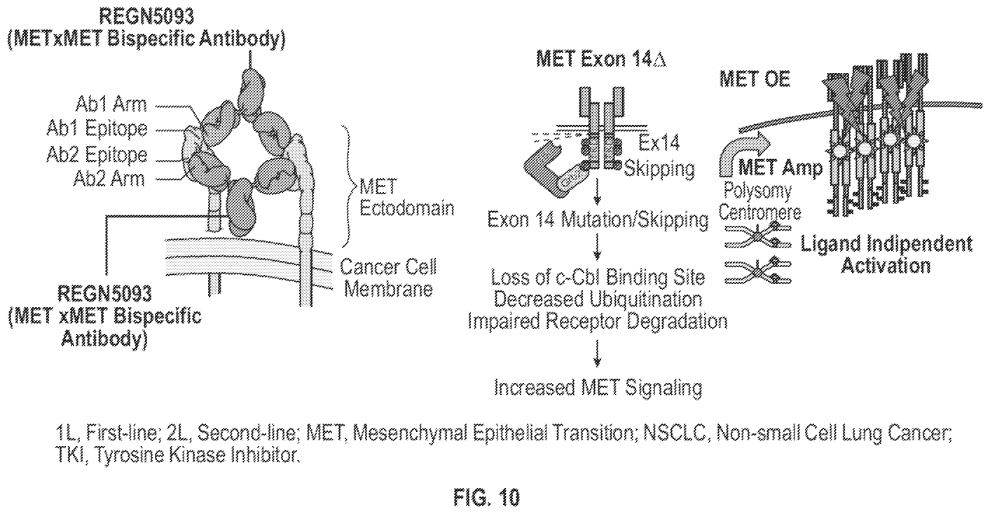

Provided herein are selection criteria that can be used to increase the percentage of responders when treating aNSCLC. In some aspects, identification of MET actionable mutations can be used for patient selection for MET-targeted therapy in advanced (2L+, i.e., patients having received two or more prior therapies) NSCLC. MET Exon 14 Skipping/Deletion is an oncogenic driver in 1L NSCLC, causing loss of c-Cbl binding site, that impairs receptor degradation, and ultimately leads to increased MET signaling. MET gene amplification is a resistance mechanism to EGFR tyrosine kinase inhibitor (TKI) therapy in 2L+ NSCLC. MET protein overexpression enriches but does not select for treatment response to MET TKIs especially in MET amplified TKI resistance-driven NSCLC populations (FIG. 10).

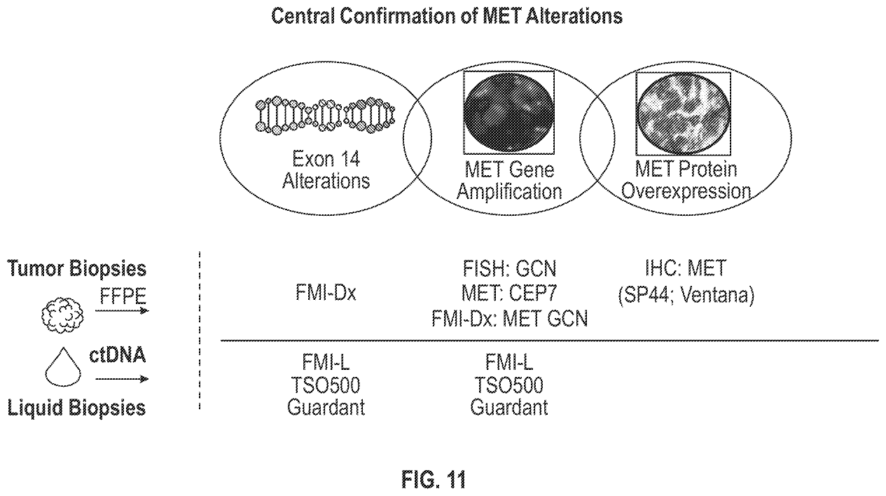

Both tumor biopsies and liquid biopsies can be used to identify and confirm MET alterations. Exon 14 alterations can be determined via gene sequencing of a tumor biopsy or ctDNA obtained from a blood sample. Similarly, MET gene amplification can be assessed via gene sequencing or fluorescence in situ hybridization [FISH; GCN or MET:Chromosome 7 centromere (CEP7) ratio] of a tumor biopsy, or sequencing of ctDNA obtained from a blood sample. MET protein expression can be assessed in tumor biopsies by immunohistochemistry using, for example, a specific c-MET antibody to stain for total MET protein (FIG. 11).

In some instances, assessing ctDNA complements tissue profiling and overcomes the limitations of biopsy collection and analysis. For example, some tumors are located in areas where a tissue biopsy would be difficult or impossible to obtain. ctDNA assessment captures all active “drivers” from all tumor sources in the body, i.e., both primary and metastatic tumors, and permits identification of mutations not present in a single tissue biopsy to overcome spatial heterogeneity. As tumors evolve over time, either or both intrinsically or in response to therapy, a higher concordance and accurate coverage can be obtained by assessing tissue and ctDNA samples obtained simultaneously to overcome temporal heterogeneity.

MET Amplification status was assessed by FISH and NGS using tissue and/or ctDNA, for example, using a threshold of Gene Copy Number (GCN) in tissues either by Fluorescent In situ Hybridization (FISH) (GCN ≥5) or NGS (GCN ≥6) and/or in ctDNA ≥2.2x by NGS assays to assess efficacy for other MET inhibitors.

MET Exon14 was centrally confirmed (i.e., assayed by one central laboratory to control any variabilities arising from sample collection and assay implementation) using various next generation sequencing (NGS) panels for tissues and ctDNA (FoundationOne® CDx (tissue based 324 gene panel) and FoundationOne® LiquidCDx (blood based 324 gene panel) are exemplary gene panels, though others are contemplated as useful herein). Exemplary Exon 14 alterations include but are not limited to D1010N, D1010fs*19, D1010Y, D1010H, or R1004P mutations and Exon 14 skipping.

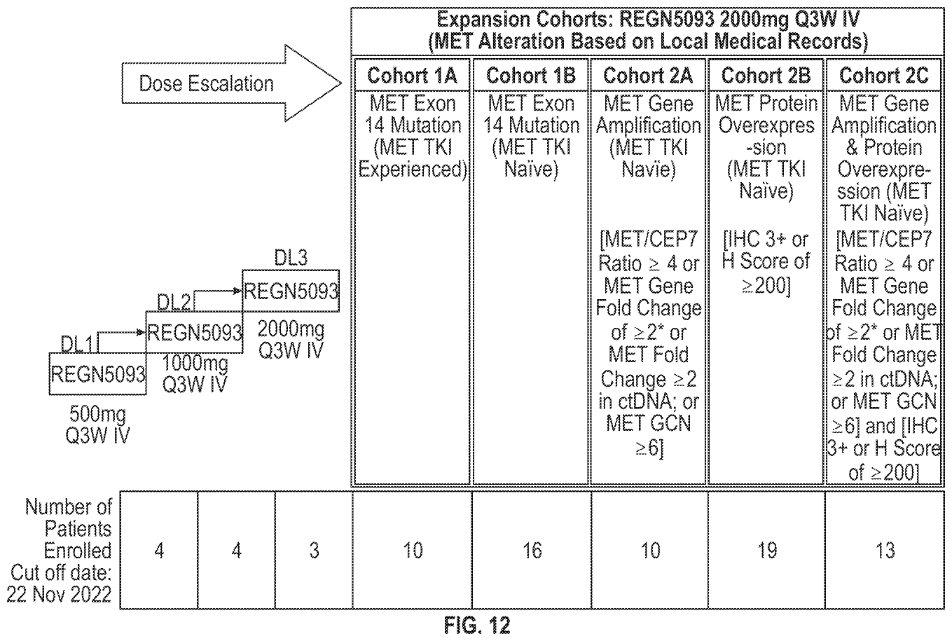

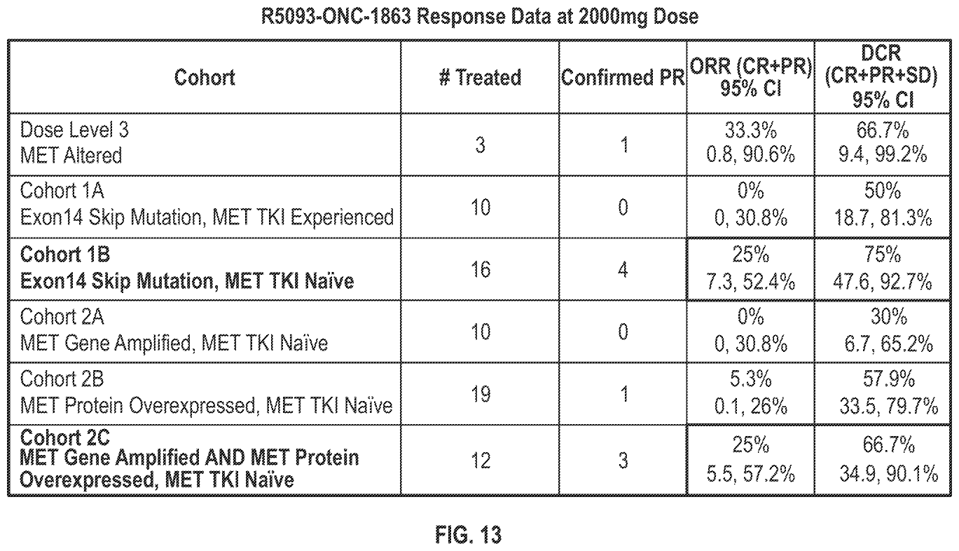

FIG. 12 provides the study design. The various cohorts had included patients with confirmed MET alterations as determined by a given patient's medical records. As shown in FIG. 12, patient enrollment during the expansion phase of the trial was based on documented MET alterations (MET Exon 14 alteration in DNA or a deletion that leads to exon 14 skipping, MET gene amplification and/or MET protein overexpression) and correlated to clinical response. As can be seen in FIG. 13, among the 65 patients who received the 2000 mg dose of REGN5093, 9 patients had a partial response. The responder distribution by cohort was as follows: Cohort 1A (MET Ex 14 TKI Experienced): 0; Cohort 1B (MET Ex 14 TKI naïve: 4; Cohort 2A (MET Amplification): 0; Cohort 2B (MET Overexpression): 1; Cohort 2C (MET Amplification and Overexpression): 3; DL3 (Dose Level 3, MET Amp and OE): 1.

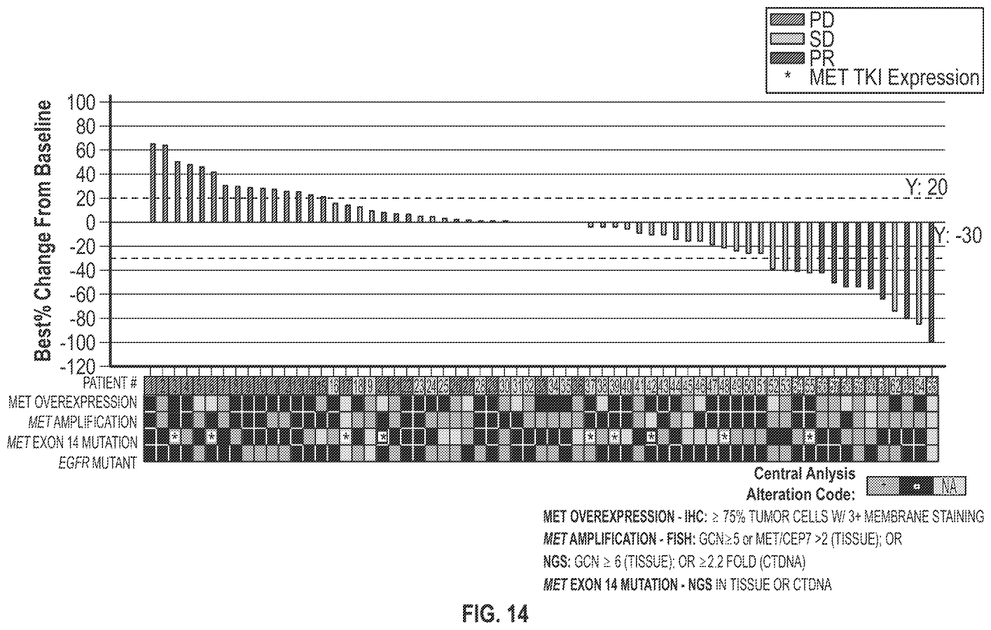

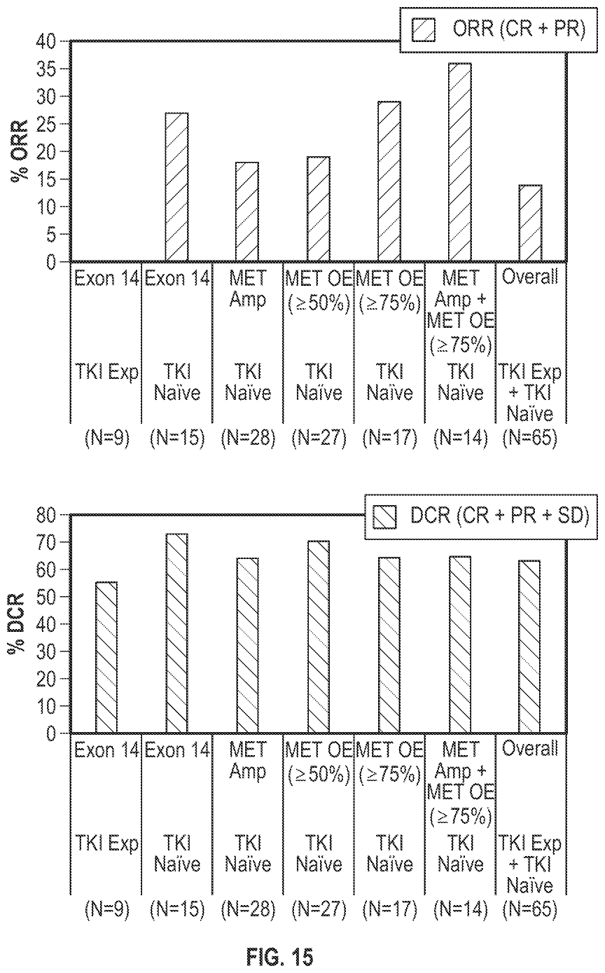

As shown in FIG. 14, tumor response was observed in MET TKI naïve patients with centrally confirmed (1) MET Exon14 (4/15) or 27% or (2) MET Amp +OE (5/14) 36% regardless of EGFR mutation status. As shown in FIG. 15, overall response rates (%ORR) were higher in specific MET altered subgroups vs overall population, by central analysis.

### Example 10: Bypass Resistance Mutations at Baseline and Clinical Response to REGN5093

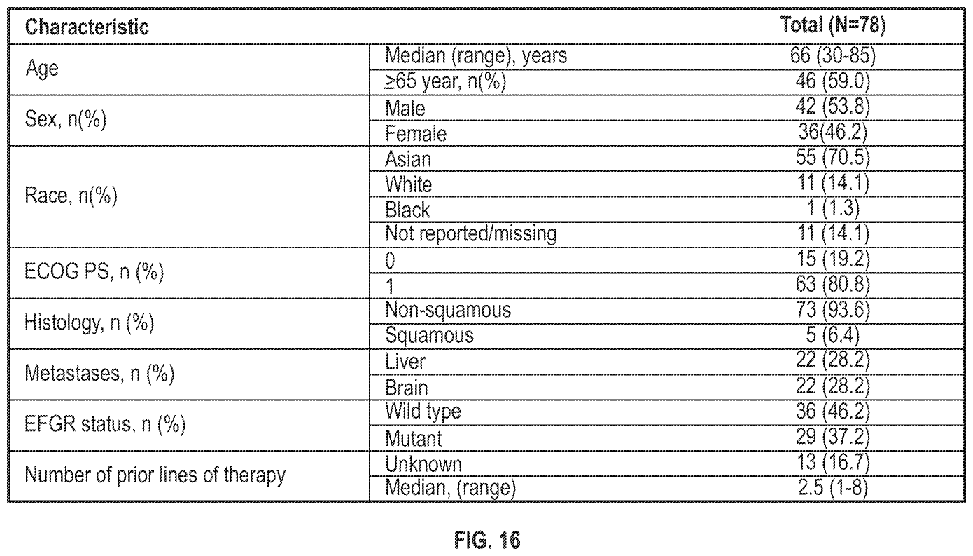

Resistance to MET therapy can be intrinsic or acquired in response to prior therapies in pre-treated 2L+NSCLC patient population. The number of therapies received by patients ranged from one to eight, and the median was 2.5 (FIG. 16). Previous therapies received by patients in the study included chemotherapy, immune checkpoint inhibitors (ICI), and EGFR inhibitors. Patients who initially respond to therapy, in some instances, acquired other mutations that promote disease progression upon treatment.

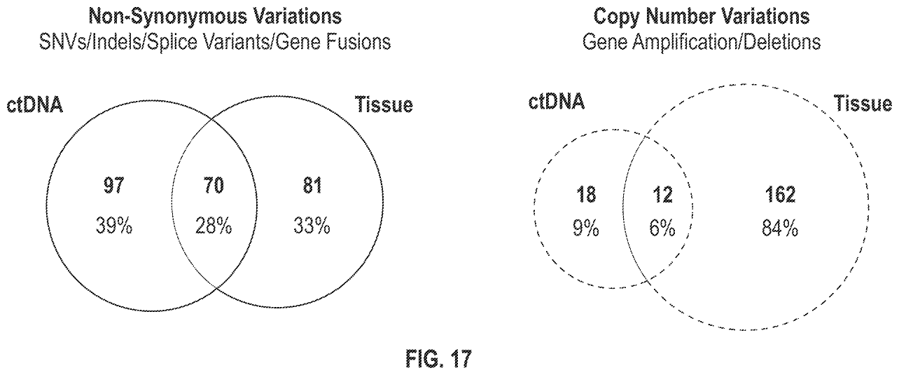

Bypass gene detection of non-synonymous variations such as single nucleotide variations (SNVs), insertion deletions (Indels), frameshifts, non-sense mutations, splice variants, or gene fusions and copy number variations (CNVs) such as gene amplifications or deletions in ctDNA supplemented tissue results and provided a more comprehensive tumor profiling of MET x MET resistance mechanism. Varying sensitivities of detection by FMI NGS panels was dependent on the variant allele frequencies (VAFs) for each of the 324 genes present in each platform. FIG. 17 illustrates the detection of non-synonymous variations and CNVs and the overlap in detection by ctDNA and tissue. NSV detection by ctDNA was 39%; in tissue, 33%; and in both, 28%. CNV detection by ctDNA was 9%; in tissue, 84%; and in both, 6%.

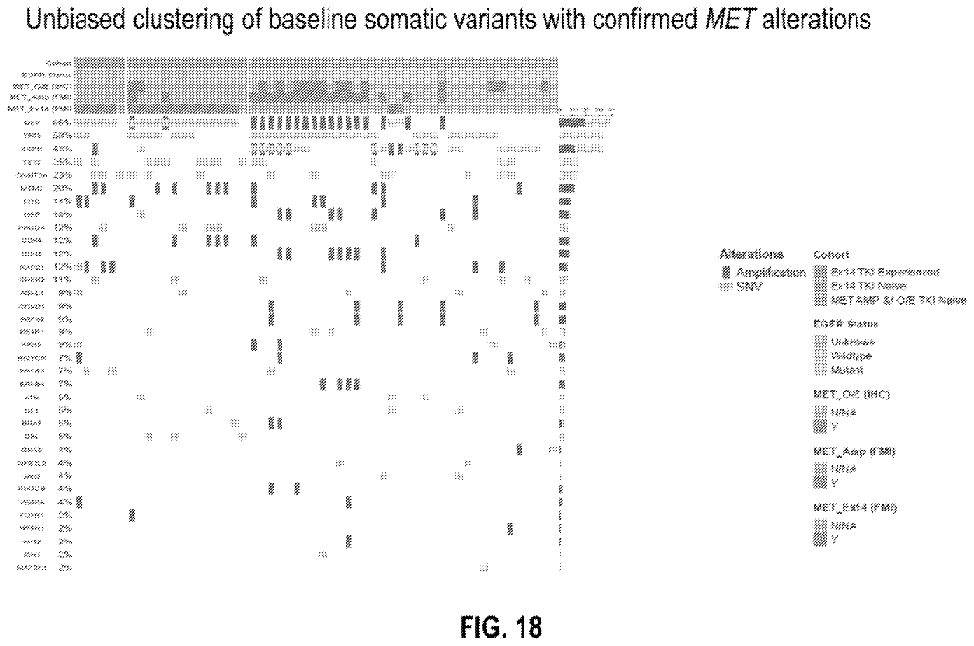

FIG. 18 illustrates unbiased co-clustering of baseline somatic mutations detected in both ctDNA and tumor tissues by cohort assignment, EGFR status, and centrally confirmed

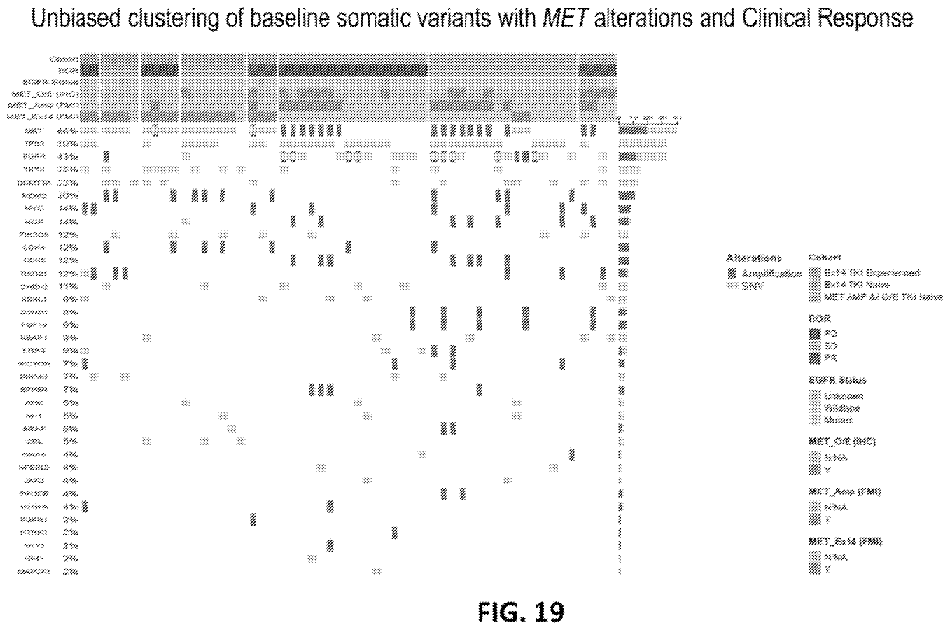

MET alterations. FIG. 19 shows another unbiased set of clustering as FIG. 18, but with clinical response. Several baseline somatic mutations based on the clustering data set were identified in non-responders that may lead to MET bypass resistance mechanisms and potentially affect clinical response to REGN5093 even in the presence of MET oncogenic drivers.

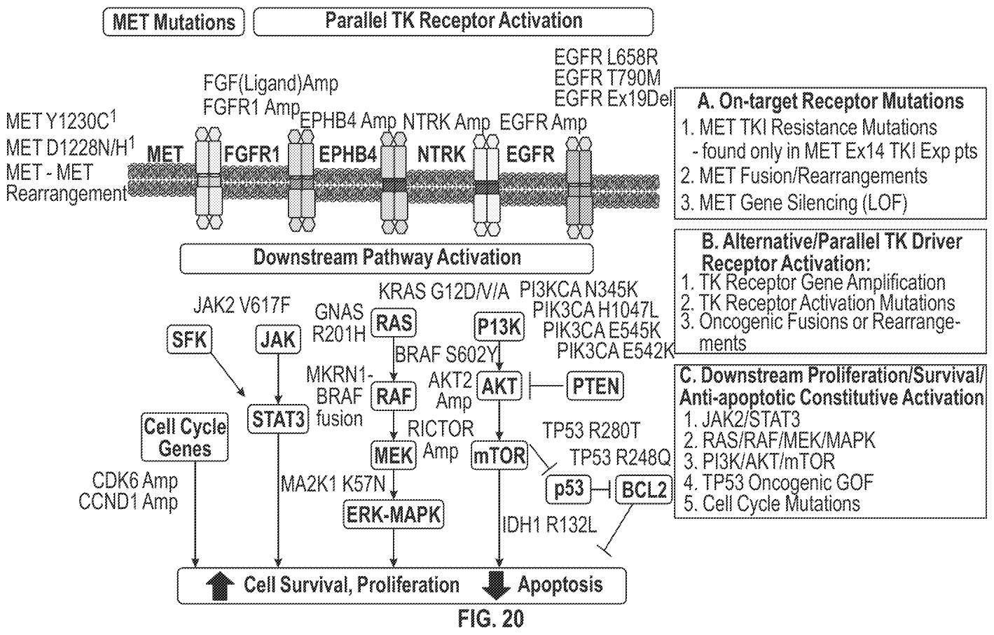

FIG. 20 provides classifications and examples of bypass alterations detected in the study patients who had centrally confirmed MET oncogenic drivers but did not respond to REGN5093. On-target receptor mutations include MET TKI resistance mutations (i.e. MET Y1230C, MET D1228H, MET D1228N), MET fusion/rearrangements (MET:MET gene rearrangement), and MET gene silencing (loss-of-function) (i.e. DNMT3A, TET2). Resistance mechanisms involving alternative or parallel tyrosine kinase driver receptor activation, include tyrosine kinase receptor (TKR) and TK ligand gene amplification (e.g., FGFR1-4 Amp, FGF14 Amp, NTRK1-3 Amp, MERTK Amp, ERBB2/3 Amp and VEGFA Amp), TKR activating mutations (EGFR L858R, G719S, E709A, E746_A750del, S752_I759del), and oncogenic fusions and rearrangements (MKRN-BRAF fusion). Resistance mechanisms involving downstream proliferation/survival/anti-apoptotic pathway activation including JAK2/STAT3 pathway (e.g., JAK2 V617F; SFKs amp); RAS/RAF/MEK/MAPK pathway (e.g., KRAS G12A/V and G12D/V; GNAS R201H, MKRN-BRAF fusion, BRAF S602Y; RICTOR Amp; MAP2K1 K57N; RAS mut/gain; RAF mut; ERK-MAPK amp), PI3K/AKT/MTOR pathway (e.g., PI3KCA H1047L, E545K, E542K, N345K; IDH1 R132L, MTOR E2338Q, AKT2 Amp, RICTOR Amp; PI3K mut; PTEN deletion); TP53 mutations (e.g., TP53 R280T, TP53 R248Q) and cell cycle mutations (e.g., CDK4 Amp, CDK6 Amp, CCND1 Amp, CCNE1 Amp).

Gene amplifications identified in patients with confirmed Met amplification and overexpression who did not respond to RENG5093 included the following: HGF, EPH, EGFR, BRAF, BCL2L1, PI3KCB, KRAS, AKT2, ATR, VEFGA, FGF, CCND, CCNE, CDK6, RAD21, and MYC. Gene deletions were also identified: CDKN2A, CDKN2B, MTAP, and RBM10. MET bypass resistance mechanisms identified in these patients are provided in Table 15.

TABLE 15
MET Bypass Resistance Mechanisms - MET Overexpression and MET Amplification

| Gene | Point Mutations or Deletions | | | | | |
|---|---|---|---|---|---|---|
| MET | MET-MET | | | | | |
| EGFR | L858R | E746_A750del | E746_s752 > V | | | |
| TP53 | C275F | R280T | G266R | R248Q | P278A | R110L |
| PI3KCA | H345K | | | | | |
| IDH1 | R132L | | | | | |
| NTRK2 | NTRK2-NTRK2 | | | | | |

Gene amplifications identified in patients with confirmed MET amplification (and not MET overexpression) were mostly EGFR mutants who did not respond to REGN5093: EGFR, BRAF, PI3KC2G, KRAS, HGF, EPHA3, ERCC4, RICTOR, RAD21, LYN, MYC, MDM2, CDK 4/6, FgF3/4/19, FGF10, and CCND1. Gene deletions in these patients included: CDKN2A, CDKN2B, MTAP, TEK, and BCOR. MET bypass resistance mechanisms identified in these patients are provided in Table 16.

TABLE 16
MET Bypass Resistance Mechanisms - MET Amplification and EGFR Mutations

| Gene | Point Mutations or Deletions | | | | | |
|---|---|---|---|---|---|---|
| EGFR | L858R | T790M | G719S | E709A | L858R | G719A |
| | R776H | S678I | E746_A750D | L861Q | I744M | |
| TP53 | H193D | H193R | V157F | R196* | R248Q | R273L |
| PI3KCA | E453K | | | | | |
| JAK2 | V617F | V615L | | | | |
| MTOR | MTOR-MTOR | | | | | |

Gene amplifications identified in patients with MET Exon 14 alterations which were TKI naïve but non-responders to REGN5093 included: MDM2, EGFR, FGFR1, ERBB3, CDK4, GNA13, MYC, RPTOR, TERC, IKZF1, EZH2, SDHA, SOX, WHSC1L1, and ZNF703. Gene deletions identified in these patients included CDKN2A, CDKN3A, and MTAP. MET bypass resistance mechanisms identified in these patients are provided in Table 17.

TABLE 17
MET Bypass Resistance Mechanisms - MET Ex 14, TKI Naïve

| Gene | Point Mutations or Deletions | | | | |
|---|---|---|---|---|---|
| EGFR | V742I | | | | |
| TP53 | R248Q | R248W | P151R | R282W | R175H |
| PI3KCA | E542K | H1047L | | | |
| MTOR | E2388Q | | | | |
| BRAF | S602Y | | | | |
| ATM | Y2019C | | | | |

Gene amplifications identified in patients with MET Exon 14 alterations which were TKI experienced but non-responders to REGN5093 included: EGFR, RAF1, PI3KC2G, CDK4, CEBPA, CDKN1A, CARD11, MYC, RICTOR, VEGFA, CD22, DDR1, RAC1, NBN, FGF19, MDM2, NFKBIA, CCND1, INPP4B, PPARG, PMS2, GATA4, SDHA, and RAD21. Gene deletions identified in these patients included CDKN2A, CDKN3A, and MTAP. MET bypass resistance mechanisms identified in these patients are provided in Table 18.

TABLE 18
MET Bypass Resistance Mechanisms -
MET Ex 14, TKI Experienced

| | |
|---|---|
| Gene | Point Mutations or Deletions |

| | | | |
|---|---|---|---|
| MET | Y1230C | D1228H | D1228N |
| TP53 | R282W | C242R | |
| PI3KCA | E545K | | |
| KRAS | G12A | G12D | |

### Example 11: Circulating Biomarkers of Target Engagement

Soluble MET (sMET) are extracellular domain fragments of MET that arises from receptors in tumor tissues that were cleaved by proteases. Total sMET and HGF were measured by ELISA.

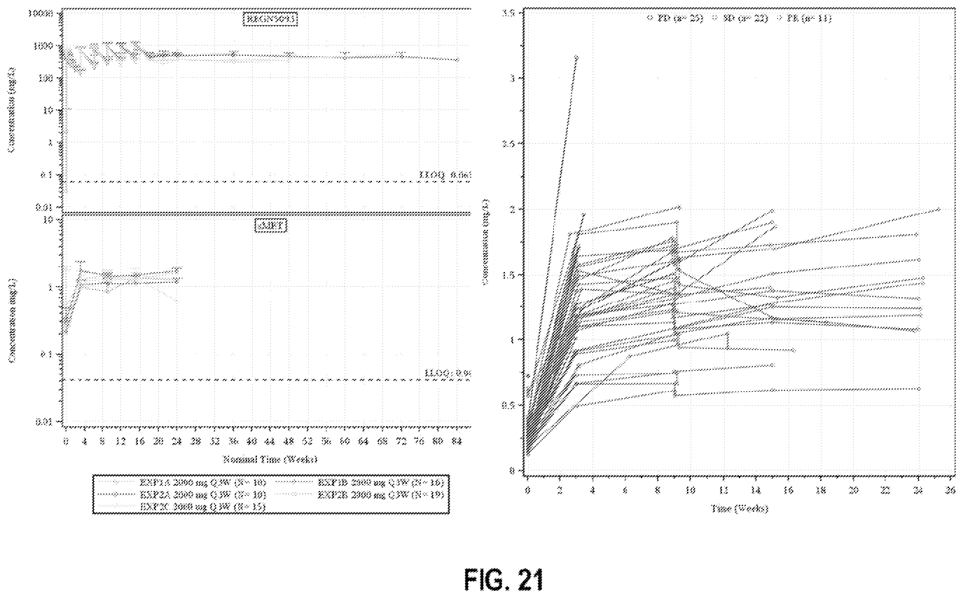

Total concentration of REGN5093 was several fold higher than total sMET concentration in serum, suggesting saturation of receptor occupancy was achieved with the 2000 mg Q3W dose regimen at Dose Expansion (FIG. 21).

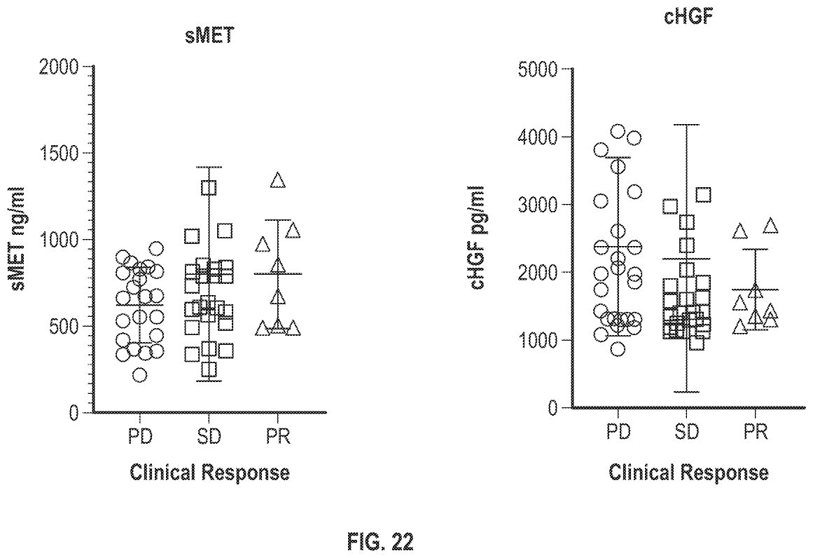

HGF is a ligand of the MET receptor that gets displaced and increases in circulation when REGN5093 binds to MET receptor in the tumor. Both circulating HGF (cHGF) and total sMET levels increased post-dose suggesting target engagement, but neither baseline nor post-treatment levels of sMET and cHGF was significantly associated with clinical response (FIG. 22).

### Conclusions

Of 36 pts who received the 2000 mg dose, 6 had a partial response (5 had prior anti-PD-(L)1 therapy). These responses occurred in 2/5 in pts with exon 14 skipping mutation who were naïve to MET tyrosine kinase inhibitor (TKI) (Cohort 1B); 0/10 in pts with exon 14 skipping mutation previously treated with TKI (Cohort 1A); and 4/21 in MET TKI-naïve pts with MET gene amplification, protein overexpression, or both (Cohorts 2A-C).

REGN5093 monotherapy can induce tumor responses in patients with MET-altered aNSCLC. Heterogeneity in response rates was observed between MET-altered subgroups in MET TKI naïve patients, with response rates of 4/15 (27%) in pts with MET Ex14 mutations and 5/16 (36%) in pts with MET Amplification and Overexpression, although based on small sample sizes.

Certain baseline somatic mutations co-occurring with MET alterations in clinical non-responders act as potential bypass resistance mechanisms and affect clinical response to REGN5093 monotherapy.

Total concentrations of REGN5093 were several fold higher than total sMET concentration in serum supporting the selection of the 2000 mg Q3W dose regimen. Total sMET and cHGF levels increased post-dose suggesting target engagement by REGN5093 but neither baseline nor post-treatment changes in total sMET nor cHGF concentrations were associated with response.

TABLE 19
Informal Sequence Listing

| SEQ | | |
|---|---|---|
| ID | | |
| NO: | Sequence | Name |
| 1 | EVQLVESGGGLVQPGTSLRLSCAASGFTFDDYAMHWVRQAPGKGLEWVSGITWN | D1-HCVR |
| | SYNIDYADSVKGRFTISRDNAKNSLYLQMNSLRAEDTALYYCAKDDDYSNYVYFDY | |
| | WGQGTLVTVSS | |
| 2 | GFTFDDYA | D1-CDR1 |
| 3 | ITWNSYNI | D1-CDR2 |
| 4 | AKDDDYSNYVYFDY | D1-CDR3 |
| 5 | EVQLVESGGGLVQPGGSLRLSCAASGFIVTTNYMTWLRQAPGKGLEWVSLIYSSGH | D2-HCVR |
| | TYYADSVKGRFTISRHNSKNTLYLQMDSLRAEDTAVYYCASAFAADVFDIWGQGTM | |
| | VTVSS | |
| 6 | GFIVTTNY | D2-CDR1 |
| 7 | IYSSGHT | D2-CDR2 |
| 8 | ASAFAADVFDI | D3-CDR3 |
| 9 | DIQMTQSPSSLSASVGDRVTITCRASQSISSYLNWYQQKPGKAPKLLIYAASSLQSGV | LCVR |
| | PSRFSGSGSGTDFTLTISSLQPEDFATYYCQQSYSTPPITFGQGTRLEIK | |
| 10 | QSISSY | LCDR1 |
| 11 | AAS | LCDR2 |
| 12 | QQSYSTPPIT | LCDR3 |
| 13 | MKAPAVLAPGILVLLFTLVQRSNGECKEALAKSEMNVNMKYQLPNFTAETPIQNVILHE | unprocessed |
| | HHIFLGATNYIYVLNEEDLQKVAEYKTGPVLEHPDCFPCQDCSSKANLSGGVWKDNIN | preproprotein of |
| | MALVVDTYYDDQLISCGSVNRGTCQRHVFPHNHTADIQSEVHCIFSPQIEEPSQCPDC | isoform “a”, |
| | VVSALGAKVLSSVKDRFINFFVGNTINSSYFPDHPLHSISVRRLKETKDGFMFLTDQSY | NCBI accession |
| | IDVLPEFRDSYPIKYVHAFESNNFIYFLTVQRETLDAQTFHTRIIRFCSINSGLHSYMEM | No. NM_ |
| | PLECILTEKRKKRSTKKEVFNILQAAYVSKPGAQLARQIGASLNDDILFGVFAQSKPDS | 001127500.2 |
| | AEPMDRSAMCAFPIKYVNDFFNKIVNKNNVRCLQHFYGPNHEHCFNRTLLRNSSGCE | |
| | ARRDEYRTEFTTALQRVDLFMGQFSEVLLTSISTFIKGDLTIANLGTSEGRFMQVVVSR | |
| | SGPSTPHVNFLLDSHPVSPEVIVEHTLNQNGYTLVITGKKITKIPLNGLGCRHFQSCS | |
| | QCLSAPPFVQCGWCHDKCVRSEECLSGTWTQQICLPAIYKVFPNSAPLEGGTRLTI | |
| | CGWDFGFRRNNKFDLKKTRVLLGNESCTLTLSESTMNTLKCTVGPAMNKHFNMSIII | |
| | SNGHGTTQYSTFSYVDPVITSISPKYGPMAGGTLLTLTGNYLNSGNSRHISIGGKTCT | |
| | LKSVSNSILECYTPAQTISTEFAVKLKIDLANRETSIFSYREDPIVYEIHPTKSFISTWW | |
| | KEPLNIVSFLFCFASGGSTITGVGKNLNSVSVPRMVINVHEAGRNFTVACQHRSNSE | |
| | IICCTTPSLQQLNLQLPLKTKAFFMLDGILSKYFDLIYVHNPVFKPFEKPVMISMGNEN | |
| | VLEIKGNDIDPEAVKGEVLKVGNKSCENIHLHSEAVLCTVPNDLLKLNSELNIEWKQA | |
| | ISSTVLGKVIVQPDQNFTGLIAGVVSISTALLLLLGFFLWLKKRKQIKDLGSELVRYDAR | |
| | VHTPHLDRLVSARSVSPTTEMVSNESVDYRATFPEDQFPNSSQNGSCRQVQYPLTD | |
| | MSPILTSGDSDISSPLLQNTVHIDLSALNPELVQAVQHVVIGPSSLIVHFNEVIGRGHFG | |
| | CVYHGTLLDNDGKKIHCAVKSLNRITDIGEVSQFLTEGIIMKDFSHPNVLSLLGICLRSE | |
| | GSPLVVLPYMKHGDLRNFIRNETHNPTVKDLIGFGLQVAKGMKYLASKKFVHRDLAAR | |
| | NCMLDEKFTVKVADFGLARDMYDKEYYSVHNKTGAKLPVKWMALESLQTQKFTTKSD | |
| | VWSFGVLLWELMTRGAPPYPDVNTFDITVYLLQGRRLLQPEYCPDPLYEVMLKCWHP | |
| | KAEMRPSFSELVSRISAIFSTFIGEHYVHVNATYVNVKCVAPYPSLLSSEDNADDEVDT | |
| | RPASFWETS | |
| 14 | MKAPAVLAPGILVLLFTLVQRSNGECKEALAKSEMNVNMKYQLPNFTAETPIQNVILHE | unprocessed |
| | HHIFLGATNYIYVLNEEDLQKVAEYKTGPVLEHPDCFPCQDCSSKANLSGGVWKDNIN | preproprotein of |
| | MALVVDTYYDDQLISCGSVNRGTCQRHVFPHNHTADIQSEVHCIFSPQIEEPSQCPDC | isoform “b”, NCBI |
| | VVSALGAKVLSSVKDRFINFFVGNTINSSYFPDHPLHSISVRRLKETKDGFMFLTDQSY | accession No. |
| | IDVLPEFRDSYPIKYVHAFESNNFIYFLTVQRETLDAQTFHTRIIRFCSINSGLHSYMEMP | NM_ |
| | LECILTEKRKKRSTKKEVFNILQAAYVSKPGAQLARQIGASLNDDILFGVFAQSKPDSAE | 000236.2 |
| | PMDRSAMCAFPIKYVNDFFNKIVNKNNVRCLQHFYGPNHEHCFNRTLLRNSSGCEAR | |
| | RDEYRTEFTTALQRVDLFMGQFSEVLLTSISTFIKGDLTIANLGTSEGRFMQVVVSRSG | |
| | PSTPHVNFLLDSHPVSPEVIVEHTLNQNGYTLVITGKKITKIPLNGLGCRHFQSCSQCLS | |
| | APPFVQCGWCHDKCVRSEECLSGTWTQQICLPAIYKVFPNSAPLEGGTRLTICGWDF | |
| | GFRRNNKFDLKKTRVLLGNESCTLTLSESTMNTLKCTVGPAMNKHFNMSIIISNGHGTT | |
| | QYSTFSYVDPVITSISPKYGPMAGGTLLTLTGNYLNSGNSRHISIGGKTCTLKSVSNSIL | |
| | ECYTPAQTISTEFAVKLKIDLANRETSIFSYREDPIVYEIHPTKSFISGGSTITGVGKNLNS | |
| | VSVPRMVINVHEAGRNFTVACQHRSNSEIICCTTPSLQQLNLQLPLKTKAFFMLDGILSK | |
| | YFDLIYVHNPVFKPFEKPVMISMGNENVLEIKGNDIDPEAVKGEVLKVGNKSCENIHLHS | |
| | EAVLCTVPNDLLKLNSELNIEWKQAISSTVLGKVIVQPDQNFTGLIAGVVSISTALLLLLGF | |
| | FLWLKKRKQIKDLGSELVRYDARVHTPHLDRLVSARSVSPTTEMVSNESVDYRATFPED | |
| | QFPNSSQNGSCRQVQYPLTDMSPILTSGDSDISSPLLQNTVHIDLSALNPELVQAVQHV | |
| | VIGPSSLIVHFNEVIGRGHFGCVYHGTLLDNDGKKIHCAVKSLNRITDIGEVSQFLTEGIIM | |
| | KDFSHPNVLSLLGICLRSEGSPLVVLPYMKHGDLRNFIRNETHNPTVKDLIGFGLQVAKG | |
| | MKYLASKKFVHRDLAARNCMLDEKFTVKVADFGLARDMYDKEYYSVHNKTGAKLPVKW | |
| | MALESLQTQKFTTKSDVWSFGVLLWELMTRGAPPYPDVNTFDITVYLLQGRRLLQPEYC | |
| | PDPLYEVMLKCWHPKAEMRPSFSELVSRISAIFSTFIGEHYVHVNATYVNVKCVAPYPSL | |
| | LSSEDNADDEVDTRPASFWETS | |
| 15 | MKAPAVLAPGILVLLFTLVQRSNGECKEALAKSEMNVNMKYQLPNFTAETPIQNVILHEH | unprocessed |
| | HIFLGATNYIYVLNEEDLQKVAEYKTGPVLEHPDCFPCQDCSSKANLSGGVWKDNINMA | preproprotein of |
| | LVVDTYYDDQLISCGSVNRGTCQRHVFPHNHTADIQSEVHCIFSPQIEEPSQCPDCVVS | isoform “c”, NCBI |
| | ALGAKVLSSVKDRFINFFVGNTINSSYFPDHPLHSISVRRLKETKDGFMFLTDQSYIDVLP | accession No. |
| | EFRDSYPIKYVHAFESNNFIYFLTVQRETLDAQTFHTRIIRFCSINSGLHSYMEMPLECILT | NM_ |
| | EKRKKRSTKKEVFNILQAAYVSKPGAQLARQIGASLNDDILFGVFAQSKPDSAEPMDRS | 001311330.1 |
| | AMCAFPIKYVNDFFNKIVNKNNVRCLQHFYGPNHEHCFNRTLLRNSSGCEARRDEYRT | |
| | EFTTALQRVDLFMGQFSEVLLTSISTFIKGDLTIANLGTSEGRFMQVVVSRSGPSTPHV | |
| | NFLLDSHPVSPEVIVEHTLNQNGYTLVITGKKITKIPLNGLGCRHFQSCSQCLSAPPFVQ | |
| | CGWCHDKCVRSEECLSGTWTQQICLPAIYKVFPNSAPLEGGTRLTICGWDFGFRRNN | |
| | KFDLKKTRVLLGNESCTLTLSESTMNTLKCTVGPAMNKHFNMSIIISNGHGTTQYSTFS | |
| | YVDPVITSISPKYGPMAGGTLLTLTGNYLNSGNSRHISIGGKTCTLKSVSNSILECYTPA | |
| | QTISTEFAVKLKIDLANRETSIFSYREDPIVYEIHPTKSFISGGSTITGVGKNLNSVSVPRM | |
| | VINVHEAGRNFTVACQHRSNSEIICCTTPSLQQLNLQLPLKTKAFFMLDGILSKYFDLIYV | |
| | HNPVFKPFEKPVMISMGNENVLEIKGNDIDPEAVKGEVLKVGNKSCENIHLHSEAVLCT | |
| | VPNDLLKLNSELNIEVGFLHSSHDVNKEASVIMLFSGLK | |
| 16 | KEALAKSEMNVNMKYQLPNFTAETPIQNVILHEHHIFLGATNYIYVLNEEDLQKVAEYKT | mature protein |
| | GPVLEHPDCFPCQDCSSKANLSGGVWKDNINMALVVDTYYDDQLISCGSVNRGTCQR | comprising the |
| | HVFPHNHTADIQSEVHCIFSPQIEEPSQCPDCVVSALGAKVLSSVKDRFINFFVGNTINS | cytoplasmic |
| | SYFPDHPLHSISVRRLKETKDGFMFLTDQSYIDVLPEFRDSYPIKYVHAFESNNFIYFLT | alpha subunit |
| | VQRETLDAQTFHTRIIRFCSINSGLHSYMEMPLECILTEKRKKR | |
| 17 | STKKEVFNILQAAYVSKPGAQLARQIGASLNDDILFGVFAQSKPDSAEPMDRSAMCAF | transmembrane |
| | PIKYVNDFFNKIVNKNNVRCLQHFYGPNHEHCFNRTLLRNSSGCEARRDEYRTEFTTA | beta subunit, |
| | LQRVDLFMGQFSEVLLTSISTFIKGDLTIANLGTSEGRFMQVVVSRSGPSTPHVNFLLD | isoform a |
| | SHPVSPEVIVEHTLNQNGYTLVITGKKITKIPLNGLGCRHFQSCSQCLSAPPFVQCGW | |
| | CHDKCVRSEECLSGTWTQQICLPAIYKVFPNSAPLEGGTRLTICGWDFGFRRNNKFD | |
| | LKKTRVLLGNESCTLTLSESTMNTLKCTVGPAMNKHFNMSIIISNGHGTTQYSTFSYVD | |
| | PVITSISPKYGPMAGGTLLTLTGNYLNSGNSRHISIGGKTCTLKSVSNSILECYTPAQTIS | |
| | TEFAVKLKIDLANRETSIFSYREDPIVYEIHPTKSFISTWWKEPLNIVSFLFCFASGGSTIT | |
| | GVGKNLNSVSVPRMVINVHEAGRNFTVACQHRSNSEIICCTTPSLQQLNLQLPLKTKA | |
| | FFMLDGILSKYFDLIYVHNPVFKPFEKPVMISMGNENVLEIKGNDIDPEAVKGEVLKVGN | |
| | KSCENIHLHSEAVLCTVPNDLLKLNSELNIEWKQAISSTVLGKVIVQPDQNFTGLIAGVV | |
| | SISTALLLLLGFFLWLKKRKQIKDLGSELVRYDARVHTPHLDRLVSARSVSPTTEMVSNE | |
| | SVDYRATFPEDQFPNSSQNGSCRQVQYPLTDMSPILTSGDSDISSPLLQNTVHIDLSAL | |
| | NPELVQAVQHVVIGPSSLIVHFNEVIGRGHFGCVYHGTLLDNDGKKIHCAVKSLNRITDI | |
| | GEVSQFLTEGIIMKDFSHPNVLSLLGICLRSEGSPLVVLPYMKHGDLRNFIRNETHNPTV | |
| | KDLIGFGLQVAKGMKYLASKKFVHRDLAARNCMLDEKFTVKVADFGLARDMYDKEYYS | |
| | VHNKTGAKLPVKWMALESLQTQKFTTKSDVWSFGVLLWELMTRGAPPYPDVNTFDITV | |
| | YLLQGRRLLQPEYCPDPLYEVMLKCWHPKAEMRPSFSELVSRISAIFSTFIGEHYVHVN | |
| | ATYVNVKCVAPYPSLLSSEDNADDEVDTRPASFWETS | |
| 18 | TKKEVFNILQAAYVSKPGAQLARQIGASLNDDILFGVFAQSKPDSAEPMDRSAMCAFPI | transmembrane |
| | KYVNDFFNKIVNKNNVRCLQHFYGPNHEHCFNRTLLRNSSGCEARRDEYRTEFTTALQ | beta subunit, |
| | RVDLFMGQFSEVLLTSISTFIKGDLTIANLGTSEGRFMQVVVSRSGPSTPHVNFLLDSHP | isoform b |
| | VSPEVIVEHTLNQNGYTLVITGKKITKIPLNGLGCRHFQSCSQCLSAPPFVQCGWCHDK | |
| | CVRSEECLSGTWTQQICLPAIYKVFPNSAPLEGGTRLTICGWDFGFRRNNKFDLKKTRV | |
| | LLGNESCTLTLSESTMNTLKCTVGPAMNKHFNMSIIISNGHGTTQYSTFSYVDPVITSISP | |
| | KYGPMAGGTLLTLTGNYLNSGNSRHISIGGKTCTLKSVSNSILECYTPAQTISTEFAVKLK | |
| | IDLANRETSIFSYREDPIVYEIHPTKSFISGGSTITGVGKNLNSVSVPRMVINVHEAGRNFT | |
| | VACQHRSNSEIICCTTPSLQQLNLQLPLKTKAFFMLDGILSKYFDLIYVHNPVFKPFEKPV | |
| | MISMGNENVLEIKGNDIDPEAVKGEVLKVGNKSCENIHLHSEAVLCTVPNDLLKLNSELNI | |
| | EWKQAISSTVLGKVIVQPDQNFTGLIAGVVSISTALLLLLGFFLWLKKRKQIKDLGSELVRY | |
| | DARVHTPHLDRLVSARSVSPTTEMVSNESVDYRATFPEDQFPNSSQNGSCRQVQYPLT | |
| | DMSPILTSGDSDISSPLLQNTVHIDLSALNPELVQAVQHVVIGPSSLIVHFNEVIGRGHFG | |
| | CVYHGTLLDNDGKKIHCAVKSLNRITDIGEVSQFLTEGIIMKDFSHPNVLSLLGICLRSEGS | |
| | PLVVLPYMKHGDLRNFIRNETHNPTVKDLIGFGLQVAKGMKYLASKKFVHRDLAARNCM | |
| | LDEKFTVKVADFGLARDMYDKEYYSVHNKTGAKLPVKWMALESLQTQKFTTKSDVWSF | |
| | GVLLWELMTRGAPPYPDVNTFDITVYLLQGRRLLQPEYCPDPLYEVMLKCWHPKAEMR | |
| | PSFSELVSRISAIFSTFIGEHYVHVNATYVNVKCVAPYPSLLSSEDNADDEVDTRPASFWE | |
| | TS | |
| 19 | TKKEVFNILQAAYVSKPGAQLARQIGASLNDDILFGVFAQSKPDSAEPMDRSAMCAFP | transmembrane |
| | IKYVNDFFNKIVNKNNVRCLQHFYGPNHEHCFNRTLLRNSSGCEARRDEYRTEFTTA | beta subunit, |
| | LQRVDLFMGQFSEVLLTSISTFIKGDLTIANLGTSEGRFMQVVVSRSGPSTPHVNFLL | isoform c |
| | DSHPVSPEVIVEHTLNQNGYTLVITGKKITKIPLNGLGCRHFQSCSQCLSAPPFVQCG | |
| | WCHDKCVRSEECLSGTWTQQICLPAIYKVFPNSAPLEGGTRLTICGWDFGFRRNNK | |
| | FDLKKTRVLLGNESCTLTLSESTMNTLKCTVGPAMNKHFNMSIIISNGHGTTQYSTFS | |
| | YVDPVITSISPKYGPMAGGTLLTLTGNYLNSGNSRHISIGGKTCTLKSVSNSILECYT | |
| | PAQTISTEFAVKLKIDLANRETSIFSYREDPIVYEIHPTKSFISGGSTITGVGKNLNSVS | |
| | VPRMVINVHEAGRNFTVACQHRSNSEIICCTTPSLQQLNLQLPLKTKAFFMLDGILSK | |
| | YFDLIYVHNPVFKPFEKPVMISMGNENVLEIKGNDIDPEAVKGEVLKVGNKSCENIH | |
| | LHSEAVLCTVPNDLLKLNSELNIEVGFLHSSHDVNKEASVIMLFSGLK | |
| 20 | TKKEVFNILQAAYVSKPGAQLARQIGASLNDDILFGVFAQSKPDSAEPMDRSAM | hMet.mmh |
| | CAFPIKYVNDFFNKIVNKNNVRCLQHFYGPNHEHCFNRTLLRNSSGCEARRDE | |
| | YRTEFTTALQRVDLFMGQFSEVLLTSISTFIKGDLTIANLGTSEGRFMQVVVSRS | |
| | GPSTPHVNFLLDSHPVSPEVIVEHTLNQNGYTLVITGKKITKIPLNGLGCRHFQS | |
| | CSQCLSAPPFVQCGWCHDKCVRSEECLSGTWTQQICLPAIYKVFPNSAPLEGG | |
| | TRLTICGWDFGFRRNNKFDLKKTRVLLGNESCTLTLSESTMNTLKCTVGPAMN | |
| | KHFNMSIIISNGHGTTQYSTFSYVDPVITSISPKYGPMAGGTLLTLTGNYLNSGN | |
| | SRHISIGGKTCTLKSVSNSILECYTPAQTISTEFAVKLKIDLANRETSIFSYREDPI | |
| | VYEIHPTKSFISTWWKEPLNIVSFLFCFASGGSTITGVGKNLNSVSVPRMVINVH | |
| | EAGRNFTVACQHRSNSEIICCTTPSLQQLNLQLPLKTKAFFMLDGILSKYFDLIY | |
| | VHNPVFKPFEKPVMISMGNENVLEIKGNDIDPEAVKGEVLKVGNKSCENIHLH | |
| | SEAVLCTVPNDLLKLNSELNIEWKQAEQKLISEEDLEQKLISEEDLHHHHHH | |
| 21 | TKKEVFNILQAAYVSKPGAQLARQIGASLNDDILFGVFAQSKPDSAEPMDRSAM | hMet.mFc |
| | CAFPIKYVNDFFNKIVNKNNVRCLQHFYGPNHEHCFNRTLLRNSSGCEARRDEY | |
| | RTEFTTALQRVDLFMGQFSEVLLTSISTFIKGDLTIANLGTSEGRFMQVVVSRSGP | |
| | STPHVNFLLDSHPVSPEVIVEHTLNQNGYTLVITGKKITKIPLNGLGCRHFQSCSQ | |
| | CLSAPPFVQCGWCHDKCVRSEECLSGTWTQQICLPAIYKVFPNSAPLEGGTRLT | |
| | ICGWDFGFRRNNKFDLKKTRVLLGNESCTLTLSESTMNTLKCTVGPAMNKHFNM | |
| | SIIISNGHGTTQYSTFSYVDPVITSISPKYGPMAGGTLLTLTGNYLNSGNSRHISIG | |
| | GKTCTLKSVSNSILECYTPAQTISTEFAVKLKIDLANRETSIFSYREDPIVYEIHPTK | |
| | SFISTWWKEPLNIVSFLFCFASGGSTITGVGKNLNSVSVPRMVINVHEAGRNFTV | |
| | ACQHRSNSEIICCTTPSLQQLNLQLPLKTKAFFMLDGILSKYFDLIYVHNPVFKPFE | |
| | KPVMISMGNENVLEIKGNDIDPEAVKGEVLKVGNKSCENIHLHSEAVLCTVPNDLL | |
| | KLNSELNIEWKQAMEVSSVFIFPPKPKDVLTITLTPKVTCVVVDISKDDPEVQFSW | |
| | FVDDVEVHTAQTQPREEQFNSTFRSVSELPIMHQDWLNGKEFKCRVNSAAFPAP | |
| | IEKTISKTKGRPKAPQVYTIPPPKEQMAKDKVSLTCMITDFFPEDITVEWQWNGQP | |
| | AENYKNTQPIMDTDGSYFVYSKLNVQKSNWEAGNTFTCSVLHEGLHNHHTEKSL | |
| | SHSPGKE | |
| 22 | ECKEALAKSEMNVNMKYQLPNFTAETPIQNVILHEHHIFLGATNYIYVLNEEDLQK | human Met |
| | VAEYKTGPVLEHPDCFPCQDCSSKANLSGGVWKDNINMALVVDTYYDDQLISCG | isoform 1 |
| | SVNRGTCQRHVFPHNHTADIQSEVHCIFSPQIEEPSQCPDCVVSALGAKVLSSVK | (Uniprot ID: |
| | DRFINFFVGNTINSSYFPDHPLHSISVRRLKETKDGFMFLTDQSYIDVLPEFRDSY | P08581) |
| | PIKYVHAFESNNFIYFLTVQRETLDAQTFHTRIIRFCSINSGLHSYMEMPLECILTE | expressed with a |
| | KRKKRSTKKEVFNILQAAYVSKPGAQLARQIGASLNDDILFGVFAQSKPDSAEPM | myc-myc- |
| | DRSAMCAFPIKYVNDFFNKIVNKNNVRCLQHFYGPNHEHCFNRTLLRNSSGCEA | hexahistidine |
| | RRDEYRTEFTTALQRVDLFMGQFSEVLLTSISTFIKGDLTIANLGTSEGRFMQVVV | (.mmh) tag |
| | SRSGPSTPHVNFLLDSHPVSPEVIVEHTLNQNGYTLVITGKKITKIPLNGLGCRHF | |
| | QSCSQCLSAPPFVQCGWCHDKCVRSEECLSGTWTQQICLPAIYKVFPNSAPLE | |
| | GGTRLTICGWDFGFRRNNKFDLKKTRVLLGNESCTLTLSESTMNTLKCTVGPAM | |
| | NKHFNMSIIISNGHGTTQYSTFSYVDPVITSISPKYGPMAGGTLLTLTGNYLNSGN | |
| | SRHISIGGKTCTLKSVSNSILECYTPAQTISTEFAVKLKIDLANRETSIFSYREDPIV | |
| | YEIHPTKSFISGGSTITGVGKNLNSVSVPRMVINVHEAGRNFTVACQHRSNSEIIC | |
| | CTTPSLQQLNLQLPLKTKAFFMLDGILSKYFDLIYVHNPVFKPFEKPVMISMGNEN | |
| | VLEIKGNDIDPEAVKGEVLKVGNKSCENIHLHSEAVLCTVPNDLLKLNSELNIEWK | |
| | QAISSTVLGKVIVQPDQNFTEQKLISEEDLGGEQKLISEEDLHHHHHH | |
| 23 | VRRLKETKDGFMF | AA 192-204 of |
| | | SEQ ID NO: 22 |
| 24 | LARQIGASLND | AA 305-315 of |
| | | SEQ ID NO: 22 |
| 25 | FIKGDLTIANLGTSEGRFMQVVVSRSGPSTPHVNF | AA 421-455 of |
| | | SEQ ID NO: 22 |

What is claimed is:

1. A method of treating non-small cell lung cancer (NSCLC), reducing NSCLC tumor growth, and or causing regression of NSCLC in a subject suffering from a tumor harboring a MET alteration, the method comprising administering to the subject a dose of about 250 to 2000 mg of a bispecific antibody comprising:
a first antigen-binding domain (D1) comprising three heavy chain complementarity determining regions (HCDR1, HCDR2 and HCDR3) within a heavy chain variable region (HCVR) comprising the amino acid sequence of SEQ ID NO: 1 and three light chain complementarity determining regions (LCDR1, LCDR2 and LCDR3) within a light chain variable region (LCVR) comprising the amino acid sequence of SEQ ID NO: 9; and
a second antigen-binding domain (D2) comprising three heavy chain complementarity determining regions (HCDR1, HCDR2 and HCDR3) within a heavy chain variable region (HCVR) comprising the amino acid sequence of SEQ ID NO: 5 and three light chain complementarity determining regions (LCDR1, LCDR2 and LCDR3) within a light chain variable region (LCVR) comprising the amino acid sequence of SEQ ID NO: 9;
wherein D1 specifically binds a first epitope of human MET; and
wherein D2 specifically binds a second epitope of human MET.

2. The method of claim 1, wherein the subject is selected as having one or more of the following criteria:
(i) MET tyrosine kinase inhibitor (TKI) naïve;
(ii) histologically confirmed NSCLC;
(iii) MET-exon14 alteration in DNA or a deletion that leads to exon 14 skipping;
(iv) MET gene amplification;
(v) elevated MET protein expression (IHC ≥2+ or H score of >150);
(vi) MET exon 14 alteration in DNA or a deletion that leads to exon 14 skipping and MET TKI experienced;
(vii) MET exon 14 alteration in DNA or a deletion that leads to exon 14 skipping and MET TKI naïve;
(viii) MET gene highly amplified (MET GCN ≥5 and/or MET/CEP7 ratio ≥2 by FISH or MET GCN ≥6 by NGS in tissues or MET fold change ≥2 in ctDNA) and MET TKI naïve;
(ix) MET protein highly overexpressed (IHC 3+ or H score of ≥200) and MET TKI naïve; and/or po1 (x) MET gene highly amplified (MET GCN ≥5 and/or MET/CEP7 ratio ≥2 by FISH or MET GCN ≥6 by NGS in tissues or MET fold change ≥2 in ctDNA), MET protein highly overexpressed (IHC 3+ or H score of ≥200), and MET TKI naïve.

3. The method of claim 1, wherein the MET alteration is an exon 14 alteration in DNA, a MET gene amplification, or MET protein overexpression.

4.-9. (canceled)

10. The method of claim 1, wherein the MET alteration is identified in a tissue sample obtained from the patient or using ctDNA from a blood sample obtained from the patient prior to treatment.

11. (canceled)

12. The method of claim 1, wherein the subject has received prior anti-cancer therapy comprising one or more of a PD-1 inhibitor, an EGFR inhibitor, a PD-L1 inhibitor, surgery, radiation therapy, or chemotherapy.

13. - 17. (Cancelled)

18. The method of claim 1 [[17]], wherein the tumor has one or more EGFR mutation [[is]] selected from the group consisting of L858R, G719S, E709A, E746_A750del, and S752_I759del.

19. The method of claim 1, wherein the tumor is non-squamous NSCLC.

20. The method of claim 1, wherein the tumor is squamous NSCLC.

21. The method of claim 1, wherein the NSCLC is metastatic.

22. The method of claim 21, wherein the NSCLC has metastasized to the brain.

23. The method of claim 21, wherein the NSCLC has metastasized to the liver.

24. The method of claim 1, wherein the NSCLC is unresectable.

25. The method of claim 1, wherein D1 comprises an HCDR1 amino acid sequence as set forth in SEQ ID NO: 2; an HCDR2 amino acid sequence as set forth in SEQ ID NO: 3; an HCDR3 amino acid sequence as set forth in SEQ ID NO: 4; an LCDR1 amino acid sequence as set forth in SEQ ID NO: 10; an LCDR2 amino acid sequence as set forth in SEQ ID NO: 11; and an LCDR3 amino acid sequence as set forth in SEQ ID NO: 12; and wherein D2 comprises an HCDR1 amino acid sequence as set forth in SEQ ID NO: 6; an HCDR2 amino acid sequence as set forth in SEQ ID NO: 7; an HCDR3 amino acid sequence as set forth in SEQ ID NO: 8; an LCDR1 amino acid sequence as set forth in SEQ ID NO: 10; an LCDR2 amino acid sequence as set forth in SEQ ID NO: 11; and an LCDR3 amino acid sequence as set forth in SEQ ID NO: 12.

26.-32. (canceled)

32. The method of claim 1, wherein the bispecific antibody is administered at a dose of 2000 mg.

33. The method of claim 1, wherein the bispecific antibody is administered intravenously, subcutaneously, or intraperitoneally.

34. The method of claim 1, wherein the bispecific antibody is administered once every three weeks.

35. (canceled)

36. The method of claim 1, wherein the treatment produces a therapeutic effect selected from the group consisting of delay in tumor growth, reduction in tumor cell number, reduction in metastasis, tumor regression, increase in survival, partial response, and complete response.

37. The method of claim 36, wherein tumor growth is delayed by at least 10 days as compared to an untreated subject.

38. The method of claim 36, wherein the tumor growth is inhibited by at least 50% as compared to an untreated subject.

39.-57. (canceled)

58. A method for identifying a candidate for Met x Met anti-tumor therapy, the method comprising obtaining a tissue sample and/or a fluid sample from a subject having NSCLC; and assessing the tissue sample and/or fluid sample for a MET alteration selected from the group consisting of an Exon 14 alteration in DNA or a deletion that leads to Exon 14 skipping, MET gene amplification, and/or Met protein overexpression,
wherein presence of at least one Met alteration in the tissue sample or fluid sample identifies the subject as a candidate for anti-tumor therapy; and
wherein the Met x Met anti-tumor therapy comprises a bispecific antibody comprising a first antigen-binding domain (D1) comprising three heavy chain complementarity determining regions (HCDR1, HCDR2 and HCDR3) within a heavy chain variable region (HCVR) comprising the amino acid sequence of SEQ ID NO: 1 and three light chain complementarity determining regions (LCDR1, LCDR2 and LCDR3) within a light chain variable region (LCVR) comprising the amino acid sequence of SEQ ID NO: 9; and
a second antigen-binding domain (D2) comprising three heavy chain complementarity determining regions (HCDR1, HCDR2 and HCDR3) within a heavy chain variable region (HCVR) comprising the amino acid sequence of SEQ ID NO: 5 and three light chain complementarity determining regions (LCDR1, LCDR2 and LCDR3) within a light chain variable region (LCVR) comprising the amino acid sequence of SEQ ID NO: 9;
wherein D1 specifically binds a first epitope of human MET; and
wherein D2 specifically binds a second epitope of human MET.

59.-65. (canceled)

66. A method for treating non-small cell lung cancer (NSCLC) in a subject, the method comprising:
(i) obtaining a tissue sample and/or a fluid sample from the subject and assessing the tissue sample and/or fluid sample for somatic mutations in one or more genes selected from the group consisting of:
a. an on-target MET Receptor gene mutation that confers resistance to MET TKI's found in MET Ex 14 Mut patients with prior TKI Exp and MET gene silencing (loss-of-function);
b. TK driver receptor activation selected from TK Receptor and ligand gene amplification and TKR activating mutations; and
c. an activating gene mutation in a pathway selected from the group consisting of JAK2/STAT3 pathway, RAS/RAF/MEK/MAPK pathway, PI3K/AKT/MTOR pathway, TP53 mutations, and cell cycle gene amplification;
(ii) administering a MET x MET bispecific antibody to the subject when such one or more gene mutations are absent; wherein the bispecific antibody comprises;
a first antigen-binding domain (D1) comprising three heavy chain complementarity determining regions (HCDR1, HCDR2 and HCDR3) within a heavy chain variable region (HCVR) comprising the amino acid sequence of SEQ ID NO: 1 and three light chain complementarity determining regions (LCDR1, LCDR2 and LCDR3) within a light chain variable region (LCVR) comprising the amino acid sequence of SEQ ID NO: 9; and
a second antigen-binding domain (D2) comprising three heavy chain complementarity determining regions (HCDR1, HCDR2 and HCDR3) within a heavy chain variable region (HCVR) comprising the amino acid sequence of SEQ ID NO: 5 and three light chain complementarity determining regions (LCDR1, LCDR2 and LCDR3) within a light chain variable region (LCVR) comprising the amino acid sequence of SEQ ID NO: 9;
wherein DI specifically binds a first epitope of human MET; and
wherein D2 specifically binds a second epitope of human MET; and
(iii) repeating steps (i) and (ii) over the course of treatment; wherein the the absence of a mutation in one or more genes is indicative of a better prognosis of the treatment as compared to a subject with a mutation in one or more genes.

67. The method of claim 66, wherein:
a. the on-target MET Receptor gene mutation is selected from the group consisting of MET Y1230C, MET D1228H, MET D1228N; and the MET gene silencing (loss-of-function) is selected from somatic mutations in DNMT3A and TET2;
b. the TKR activating mutation is selected from the group consisting of EGFR L858R, EGFR G719S, EGFR E709A, EGFR E746_A750del, and EGFR S752_I759del; and
c. the JAK2/STAT3 pathway mutation is JAK2 V617F; the RAS/RAF/MEK/MAPK pathway mutation is selected from the group consisting of KRAS G12A/V, GNAS R201H, MKRN-BRAF fusion, BRAF S602Y, RICTOR Amp, and MAP2K1 K57N; the PI3K/AKT/MTOR pathway mutation is selected from the group consisting of PIK3CA H1047L, PIK3CA E545K, PIK3CA E542K, PIK3CA N345K, IDH1 R132L, and MTOR E2338Q; the PI3K/AKT/MTOR pathway amplification is selected from the group consisting of AKT2 Amp and RICTOR Amp; the TP53 mutation is selected from the group consisting of TP53 R280T and TP53 R248Q; and the cell cycle gene amplification is selected from the group consisting of CDK4 Amp, CDK6 Amp, CCND1 Amp, and CCNE1 Amp.

68.-72. (canceled)
